(19) United States
(12) Patent Application Publication
(10) Pub. No.: US 2022/0214591 A1
(43) Pub. Date: Jul. 7, 2022

(54) MULTI-COLOR ELECTROCHROMIC DEVICES

(71) Applicant: YADA RESEARCH AND DEVELOPMENT CO. LTD., Rehovot (IL)

(72) Inventors: Milko E. VAN DER BOOM, Rishon Lezion (IL); Michal LAHAV, Rehovot (IL); Neta ELOOL DOV, Rehovot (IL); Naveen MALIK, Rehovot (IL)

(73) Assignee: YADA RESEARCH AND DEVELOPMENT CO. LTD., Rehovot (IL)

(21) Appl. No.: 17/606,772

(22) PCT Filed: Apr. 28, 2020
(86) PCT No.: PCT/IL2020/050478

§ 371 Date: Oct. 27, 2021

Related U.S. Application Data
(60) Provisional application No. 62/877,329, filed on Jul. 23, 2019.

(30) Foreign Application Priority Data
Apr. 28, 2019 (IL) 266281

(51) Int. Cl. G02F 1/1516; G02F 1/1524; G02F 1/153; G02F 1/163; C09K 9/02; B05D 1/02; B05D 7/00; B29D 11/00

(52) U.S. Cl. CPC G02F 1/1516; G02F 1/1524; G02F 1/153; G02F 1/163; C09K 9/02; B05D 1/02; C09K 2211/187; B05D 7/58; B29D 11/00788; B29D 11/00634; G02F 2001/1518; C09K 2211/185; C09K 2211/1029; B05D 7/54

(57) ABSTRACT

This invention relates to multi-color electrochromic devices and to methods of use thereof. The invention also relates to a process of preparation of the electrochromic devices.

### FIELD OF THE INVENTION

This invention relates to multi-color electrochromic devices and to methods of use thereof. The invention also relates to a process of preparation of the electrochromic devices.

### BACKGROUND OF THE INVENTION

Electrochromic (EC) materials have distinct ability to alter their optical transparency in response to application of voltage. This property is particularly useful in EC applications including light filtering windows, smart windows, electrochromic windows, smart mirrors, optical filters, frequency doubling devices, spatial light modulators, pulse shapers, electronic display systems such as color filter displays, monitors and TVs, signs, plastic electronics, lenses and sensors, optoelectronics systems such as optical switches for telecommunication and optical/laser systems (e.g. for machining, medical treatments, army/military/space); construction materials and products for the auto industry such as tintable reflective surfaces (e.g. car mirrors). Technology based on electrochromic properties may find use in numerous other devices and products where electrical switching of optical properties is utilized.

In recent years, electrochromic (EC) materials such as metal-oxides, conductive polymers, liquid crystals, organic molecules and polymers have been investigated. Efforts were made to develop high-performance and efficient EC devices (ECD's) and advance these materials beyond academic interest. A certain class of EC devices for more advanced use includes devices exhibiting multi-color switching. Multi-color devices expand the range of potential EC applications.

One currently-explored approach for obtaining multi-color electrochromic films is to use polymeric materials grafted with an extra/different functional group such as tri-phenyl amine or viologen. However, the colors of different chromic states in these systems are not sufficiently diversified. Moreover, they lack a sufficiently-colorless state. This deficiency restricts their application. Further, electrical switching in these devices is usually based on solution electrochemistry with low stability and durability. This further limit application of these materials.

Another promising strategy for obtaining multi-color electrochromic devices involves a color mixing concept based on the incorporation of different electrochromic materials on different working electrodes. In this technique, materials exhibiting complementary colors are deposited onto different transparent conducting electrodes (TCEs). This allows to generate a multi-color system by selectively controlling the redox reaction on each electrode using a bi-potentiostatic technique. Although this color mixing strategy allows to generate different colors, it is highly complex due to the requirement for multiple working electrodes.

Recently, a film formed by sandwiching a ruthenium-based polymer with Prussian blue (PB) nanoparticles to compose hybrid multi-stimuli-responsive thin tri-layer film was demonstrated. In this study, trapped charge in the oxidized Prussian yellow (PY) state was released successfully by photoinduced electron transfer mechanism from Ru* to Prussian yellow (PY) to regenerate the initial state. This tri-layer hybrid system incorporates three different chromic states using a single working electrode. However the film thickness was too thin to observe the color change by the naked eye and switching to the initial state was only achieved for up to 10 cycles.

An interesting class of EC materials is metal-coordinated organic complexes where a metal ion is coordinately bonded to an organic molecule (a ligand). In order to obtain high-performance films of EC materials, the materials should be coated on a conducting, transparent substrate in a uniform manner. Film composition, film thickness, film density and film uniformity are properties that can affect the EC performance of the material film. Such properties are important for implementation. Film properties depend on the film preparation method. In this area, an alternative strategy of color mixing on a single working electrode has been introduced by forming assemblies of coordinated polypyridyl metal complexes. The electron transfer properties of these coordination-based assemblies were found to be dependent on the sequence and thickness of the assembly blocks.

In view of the promising EC properties of metal-coordinated organic complexes, there is a need to find an efficient process for preparing high-performance EC materials and films comprising such complexes.

### SUMMARY OF THE INVENTION

Spray coating is a promising approach that can be combined with industrially important roll-to-roll (R2R) coating processes, which is not possible with other coating methods. Therefore, spray coating provides a step forward for making molecular assemblies (MA's) of e.g. polypyridyl metal complexes compatible with industrial processes. At the same time, spray coating allows the fabrication and functionalization of large surfaces. This is in contrast to existing methods where surface coating is limited to a small surface area.

Further in terms of processing time, and in one embodiment, when comparing the spray coating process to spin coating of a similar layer, the spray coating process is much faster, in some embodiments, two-time faster.

In one embodiment, the spray-coating method used herein is fully automated and it was used to fabricate large surface area devices.

In one embodiment, this invention provides a method of preparation of an electrochromic device, said method comprising:
a. providing a substrate;
b. applying a linker comprising a metal ion to said substrate by spray-coating, thus forming a linker layer on said substrate;
c. applying a metal-coordinated organic complex to said linker layer by spray coating, thus forming a layer of metal-coordinated organic complex on said linker layer;
d. optionally repeating steps b and c;
   thereby forming an electrochromic device comprising a substrate and comprising at least one layer of a linker and at least one layer of a metal-coordinated organic complex.

In one embodiment, the metal-coordinated organic complex comprises at least one functional group, said functional group capable of binding to the metal ion. In one embodiment, the binding comprises a coordination bond between the functional group and said metal ion.

In one embodiment, the metal-coordinated organic complex is polypyridyl complex.

In one embodiment, the spray coating steps for applying the metal linker and the organic complex are conducted at atomization pressure ranging between 0.75 kPa and 1.50 kPa. In one embodiment, the spray coating steps for applying the metal linker and the organic complex are conducted at a nozzle to substrate distance ranging between 3.0 and 8.0 cm. In one embodiment, the spray coating steps for applying the metal linker and the organic complex are conducted at a spraying solution flow rate ranging between 0.4 and 0.8 mL/min and at room temperature. Any combination of the above-mentioned parameters is included in embodiments of this invention.

In one embodiment, spraying is conducted such that the spraying nozzle is moved parallel to the substrate in a pattern along the X-Y substrate directions at a speed ranging between 3 and 7 mm/s. X-Y substrate directions are the substrate directions parallel to the sprayed surface.

In one embodiment, both applying steps (of linker and complex) are repeated to obtain from 2 to 80 (linker+organic-complex) layers.

In one embodiment, the metal ion in the linker is selected from the group consisting of Pd, Zn, Os, Ru, Fe, Pt, Ni, Ir, Rh, Co, Cu, Re, Tc, Mn, V, Nb, Ta, Hf, Zr, Cr, Mo, W, Ti, Sc, Ag, Au, and Y.

In one embodiment, this invention provides an electrochromic (EC) device, made by the method as described herein above.

In one embodiment, the metal-coordinated organic complex in said device comprises one type of metal ion. In one embodiment, the metal-coordinated organic complex in said device comprises at least two types of metal ions.

In one embodiment, the at least two types of metal ions comprise metal ions selected from Fe, Os, Ru, Co, Ni, Mn, Cu, Zn, Ti, C, Cr, Rh, or Ir.

In one embodiment, the metal-coordinated organic complex is a polypyridine complex comprising two types of metal ions, said two types are Fe and Os ions or Fe and Ru ions or Ru and Os ions.

In one embodiment, the device has a contrast ratio between an oxidized and a reduced state of at least 10%, at least 20%, at least 30%, at least 40%, at least 50% or at least 60%, or a contrast ratio ranging between 10% and 20%, between 10% and 50%, between 25% and 50%, between 10% and 40%, between 10% and 70%.

In one embodiment, the device has a contrast ratio between an oxidized and a reduced state of at least 80% or at least 90%, or a contrast ratio ranging between 50% and 90%, between 50% and 99%, between 25% and 95%, between 60% and 80%, between 75% and 100%, between 10% and 100%.

In one embodiment, the device is able to retain at least 90% of its maximum contrast ratio after 1000 switching cycles between oxidized and reduced state(s).

In one embodiment, the device further comprising a power supply and electrical connections, said electrical connections connecting said device to the power supply wherein:
a first connection connecting said substrate to a first pole of said power supply;
a second connection connecting said metal-coordinated organic complex layer directly or through intermediate layers to a second pole of said power supply.

In one embodiment, the intermediate layers comprise an electrolyte, a storage layer, a spacer or any combination thereof

In one embodiment, the electrolyte is chosen from liquid electrolyte, gel electrolyte or solid electrolyte.

In one embodiment, this invention provides a smart window comprising the device as described herein above, wherein said substrate is transparent in the visible-light range and wherein the lateral length and width of said window measured parallel to the largest surface of said substrate is ranging between 1 cm to 10 m.

In one embodiment, this invention provides a switch comprising the device as described herein above, wherein the substrate is transparent in at least a portion of the visible-light range.

In one embodiment, this invention provides an optical switch, a memory device or an encoder comprising:
the device as described herein above, wherein said substrate is transparent in at least a portion of the visible-light range;
an optical detector.

In one embodiment, this invention provides a method of changing the absorption spectrum of the device as described herein above, said method comprising:
providing a device comprising:
   a substrate;
   a first linker layer, said layer attached to said substrate;
   a first metal-coordinated organic complex layer, said metal-coordinated organic complex comprising one type of metal ion, said complex layer is attached to said linker layer;
   optionally additional alternating layers of said linker and of said metal-coordinated organic complex constructed on top of said first metal-coordinated organic complex layer;
   wherein said metal-coordinated organic complex is electrochromic such that when a certain voltage is applied to it, the oxidation state of said metal ion is changed and wherein said oxidation state change causes a change in the absorption spectrum of said metal-coordinated organic complex;
applying voltage to said device, thus changing the oxidation state of said metal ion, thereby inducing change in the absorption spectrum of said metal-coordinated organic complex, thus changing the absorption spectrum of said device.

In one embodiment, the substrate is at least partially transparent in the visible range.

In one embodiment, the voltage varies between (−3.0 V) and 3.0 V. In one embodiment, the voltage varies between (0.1 V) and 2.0 V.

In one embodiment, the change in absorption spectrum is reversible. In one embodiment, the method further comprising applying a second voltage to said device, thus changing the absorption spectrum of said device back to its initial spectrum.

In one embodiment, this invention provides a method of changing the absorption spectrum of the device as described herein above, the method comprising:
providing a device comprising:
   a substrate;
   a first linker layer, said layer attached to said substrate;
   a first metal-coordinated organic complex layer, said metal-coordinated organic complex comprising two types of metal ions, said complex layer is attached to said linker layer;
   optionally additional alternating layers of said linker and of said metal-coordinated organic complex constructed on top of said first metal-coordinated organic complex layer;
      wherein said metal-coordinated organic complex is electrochromic such that when a certain voltage is applied to it, the oxidation state of at least one type of said metal ions is changed and wherein said oxidation state change causes a change in the absorption spectrum of said metal-coordinated organic complex;
applying a first voltage to said device, thus changing the oxidation state of a first of said metal ions, thereby inducing change in the absorption spectrum of said metal-coordinated organic complex, thus changing the absorption spectrum of said device.
applying a second voltage to said device, thus changing the oxidation state of a second of said metal ions, thereby inducing an additional change in the absorption spectrum of said metal-coordinated organic complex, thus changing the absorption spectrum of the device.

In one embodiment, the substrate is at least partially transparent in the visible range.

In one embodiment, the voltage varies between (−3.0 V) and 3.0 V. In one embodiment, the voltage varies between (0.1 V) and 2.0 V.

In one embodiment, the change in absorption spectrum is reversible.

In one embodiment, the method further comprising applying a third voltage to said device, thus changing the absorption spectrum of said device back to its initial spectrum or back to its intermediate spectrum.

In one embodiment, alternating the sequence of applying the first, second and/or third voltages is used when operating devices of this invention. For example, a sequence where the voltages are applied as follows: first, second, first, third, second is one operation possibility. Other repetitions and sequence combinations are included in embodiments if this invention.

In some embodiments, the intermediate spectrum is the spectrum after applying a first voltage and prior to applying the second voltage. The initial spectrum is the spectrum before applying the first voltage.

In one embodiment, this invention provides a new strategy for the formation of stable multi-color electrochromic metallo-organic assemblies by a color mixing concept on a single working electrode, avoiding the need for multiple conducting electrodes. In some embodiments, these assemblies are of sub-micron thicknesses in some embodiments and show one colorless and two well-defined colored states upon the application of different voltages. The redox changes in the assemblies are noticeably observed by the naked eye in some embodiments. In one embodiment, these 3D-coordination network assemblies are formed by alternate spray coating of a palladium salt and a mixture of divalent polypyridyl complexes on a transparent electrode. In one embodiment, a fully automated spray-coating method is utilized (FIG. 6B). In an example, the multi-color electrochromic performance of the spray-coated assemblies was evaluated in solution (˜2000 cycles) and in laminated devices (˜1200 cycles) by switching between three different redox states using only one working electrode. The contrast found (ΔTₘₐₓ) was up to 57% in some embodiments.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 schematically shows the structure of metal complexes 4, 1 and 2.

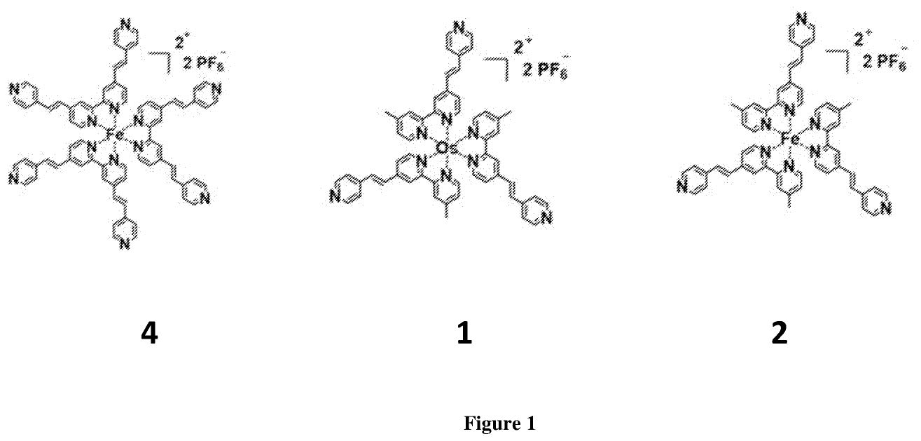

FIGS. 2A-2B schematically show composition and electrochemically addressable multi-states of coordination-based molecular assemblies (MAs). (FIG. 2A) Components used for the formation of the metallo-organic assemblies by automated-ultrasonic-spray-coating on TCOs using equimolar (0.2 mM each, DCM/MeOH, 1:1 v/v) solutions containing complexes 1·4 and a solution of (1.0-2.0 mM, THF) PdCl₂ were used. Schematic representation of electrochemical permeability of [MA1|FTO/glass], [MA4|FTO/glass] and [MA1·4|FTO/glass] films. Diffusion rate is shown (Diffusion of electrolyte ion in single- vs. bi-component MAs); (FIG. 2B) Multi-electrochromic behavior of bi-component MAs, schematic representation of the three states of multi-electrochromic [MA1·4|FTO/glass] film: state A (red color), state B (gray color) and state C (colorless color), using: an acetonitrile (ACN) solution of TBAPF₆ (0.1 M) as the supporting electrolyte, with the functionalized FTO, Pt wire, and Ag wire as the working, counter and reference electrodes, respectively.

FIGS. 3A-3L show comparison of electrochemical phenomenon of single component [MA1|FTO/glass], [MA4|FTO/glass] and multicomponent [MA1·4|FTO/glass], in a 0.1 M TBAPF₆/ACN electrolyte solution. FIGS. 3A-3D: Schematic diagram and photograph of electrochemical process of MA1 on FTO/glass (2 cm×2 cm) substrate (FIG. 3A); Reversible absorption spectral changes during spectroelectrochemical measurements corresponding to two redox states A and B of MA1 device (FIG. 3C), cyclic voltammogram (CV) of the 1st cycle (FIG. 3B) and switching time of MA1 (FIG. 3D). FIGS. 3E-3H: Schematic diagram and photograph of electrochemical process of MA4 on FTO/glass (2 cm×2 cm) substrate (FIG. 3E). Reversible absorption spectral changes during spectroelectrochemical measurements corresponding to two redox states A and B of MA4 device (FIG. 3G), cyclic voltammogram (CV) of the 1st cycle (FIG. 3F) and switching time of MA4 (FIG. 3H). FIGS. 3I-3L: Schematic diagram and photograph of electrochemical process of multicomponent MA1·4 on FTO/glass (2 cm×2 cm) substrate (FIG. 3I); Reversible absorption spectral changes during spectroelectrochemical measurements corresponding to three redox states A, B and C of MA1·4 film (FIG. 3K), cyclic voltammogram (CV) of the 1st cycle (FIG. 3J) and switching time of MA1·4 (FIG. 3L).

FIGS. 4A-4C shows: spectroelectrochemical (SEC) operation of multi-color electrochromic [MA4·1|FTO/glass] film (2 cm×2 cm) in 0.1M TBAPF₆/acetonitrile electrolyte; (FIG. 4A) Transmission spectra of the MA4·1 on FTO/glass film; corresponding to two consecutive oxidation states (from top to bottom), upon stepwise application of potentials from: (i) 0.2-0.85 V and (ii) 0.85-1.8 V; (FIG. 4B) SEC stability of the MA4·1 on FTO/glass, using double potential steps: (i) 0.2-1.8 V, (ii) 0.2-0.8 V and (iii) 0.75-1.8 V at λₘₐₓ=530 nm; (FIG. 4C) Chronoamperometric (CA) measurements using double potential steps: (i) 0.2-1.8 V, (ii) 0.2-0.8 V and (iii) 0.75-1.8 V.

FIGS. 5A-5C: Structure and operation of laminated multi-color electrochromic device. (FIG. 5A) spectroelectrochemical (SEC) performance of the MA4·1 in laminated devices on 2 cm×2 cm FTO/glass in a LiClO₄/PMMA/ACN based electrolyte using double potential steps: (i) −1.2 V to +2.8 V and (ii) −1.8 V to +2.8 V, at λₘₐₓ=530 nm; (FIG. 5B) Photographs and schematic presentation of the electrochemically addressable three-redox-state upon stepwise application of potentials in a laminated device using LiClO₄/PMMA/ACN based electrolyte and double-sided tape (3M 9088) was used as the spacer. (FIG. 5C) Chronoamperometric (CA) measurements using double potential steps: (i) −1.2 V to +2.8 V and (ii) −1.8 V to +2.8 V.

FIGS. 6A-6B: Formation and composition of coordination-based molecular assemblies (MAs). (FIG. 6A) Components used for the formation of metallo-organic assemblies. (FIG. 6B) Automated-ultrasonic-spray-coating sequence for the fabrication of the metallo-organic assemblies (MA) on TCOs. Equimolar (0.2 mM each, DCM/MeOH, 1:1 v/v) solutions containing complexes 1·4, 2·4, and 2·3 and solution of (1.0-2.0 mM, THF) PdCl₂ were used.

FIGS. 7A-7C: Mixing of polypyridine complexes to form MA1·4, MA2·4 and MA2·3 on 2 cm×2 cm FTO/glass. (FIG. 7A) Photograph of the colored and bleached state and cyclic voltammograms (CVs) of MA1, MA1·4 and MA4 (left to right). (FIG. 7B) Photograph of the colored and bleached state and cyclic voltammograms (CVs) of MA2, MA2·4 and MA4 (left to right). (FIG. 7C) Photograph of the colored and bleached state and cyclic voltammograms (CVs) of MA2, MA2·3 and MA3 (left to right). These experiments were carried out using: an acetonitrile (ACN) solution of TBAPF₆ (0.1 M) as the supporting electrolyte, with the functionalized FTO, Pt wire, and Ag wire as the working, counter and reference electrodes, respectively.

FIG. 8 Spectroelectrochemical (SEC) performance of the molecular assemblies (MA1·4) on FTO/glass (2 cm×2 cm) in a 0.1 M TBAPF₆/ACN electrolyte solution. (A) Photograph of the three-redox states of MA1·4: state A (bordeaux red), state B (gray), and state C (colorless). (B) Gradual oxidation spectral changes while changing potential from 0.2 V to 1.8 V of MA1·4 device. Bare substrates were used for the baseline (black). (C) Gradual reduction spectral changes while changing potential from 1.8 V to 0.2 V of MA1·4 device. Bare substrates were used for the baseline (black). (D) Dependence of the contrast ratio (ΔT) at different λ=480 nm, 530 nm and 590 nm, using double potential steps: 0.2 V to 1.8 V (switching between state A→C). (E) SEC switching using double potential steps: (i) 0.2 V to 1.8 V (A→C) and (ii) 0.2 V to 0.8 V (A→B) at λₘₐₓ=530 nm. (F) SEC switching using double potential steps: (i) 0.2 V to 0.8 V (A→B), (ii) 0.8 V to 1.8 V (B→C), (iii) 1.8 V to 0.7 V (C→B), and (iv) 0.7 V to 0.2 V (B→A) at λ=530 nm. (G) SEC switching using double potential steps: (i) 0.2V to 0.8 V to 0.2 V (A→B→A), (ii) 0.2 V to 1.8 V to 0.7 V (A→C→A), and (iii) 0.7 V to 1.8 to 0.2 V (B→C→A). (H) SEC while switching between different state at λₘₐₓ=530 nm, using double potential steps: (i) 0.2 V to 1.8 V (A→C), (ii) 0.2 V to 0.8 V (A→B), and (iii) 0.7 V to 1.8 V (B→C). (I) SEC switching at various pulse width between state A-C. (J) SEC stability of MA1·4 using double potential steps: (i) 0.2 V to 1.8 V (A→C) and (ii) 0.2 V to 0.8 V (A→B) at λₘₐₓ=530 nm.

FIG. 9 Comparison of electrochemical phenomenon of single component [MA4|FTO/glass], [MA2|FTO/glass] films and multicomponent [MA2·4|FTO/glass], in a 0.1 M TBAPF₆/ACN electrolyte solution. The switching times for all films, are the time taken for 90% change in ΔTₘₐₓ. ΔTₘₐₓ=Tb (fully bleached)−Tc (fully colored). (A-D) Schematic diagram and photograph of electrochemical process of MA2 on FTO/glass (2 cm×2 cm) substrate. Reversible absorption spectral changes during spectroelectrochemical measurements corresponding to two redox states A and B of MA2 film, cyclic voltammogram (CV) of the 1st cycle and switching time of MA2. (E-H), Schematic diagram and photograph of electrochemical process of MA4 on FTO/glass (2 cm×2 cm) substrate. Reversible absorption spectral changes during spectroelectrochemical measurements corresponding to two redox states A and B of MA4 film, cyclic voltammogram (CV) of the 1st cycle and switching time of MA4. (I-L) Schematic diagram and photograph of electrochemical process of multicomponent MA2·4 on FTO/glass (2 cm×2 cm) substrate. Reversible absorption spectral changes during spectroelectrochemical measurements corresponding to three redox states A, B and C of MA2·4 film, cyclic voltammogram (CV) of the 1st cycle and switching time of MA2·4.

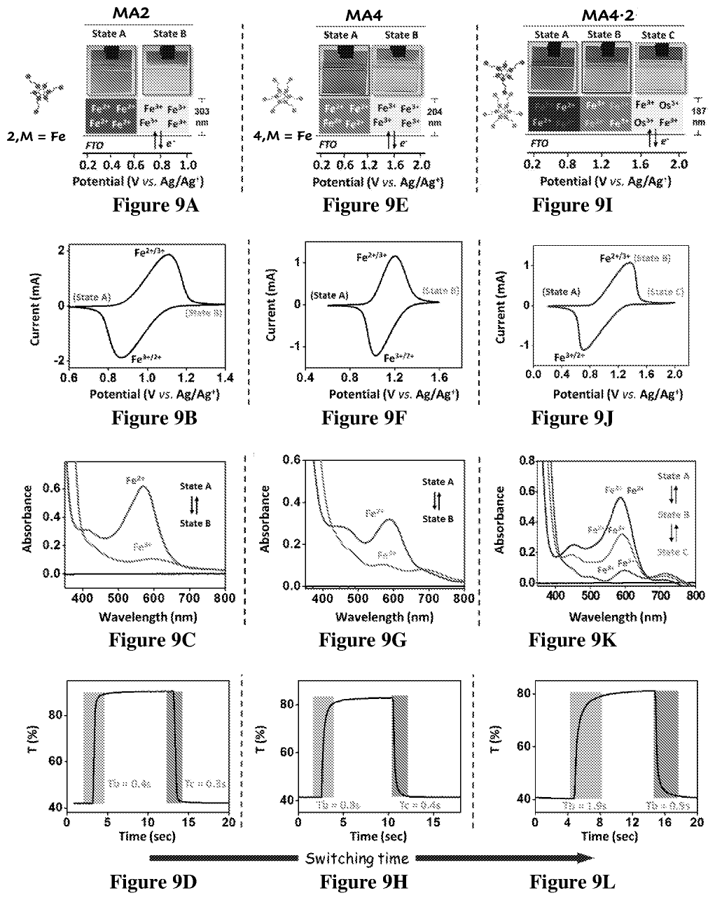

FIG. 10: Photograph of the three redox states of MA2·3: red-orange-colorless and reversible absorption spectral changes during spectroelectrochemical measurements corresponding to state A (red traces), 1^{st} oxidized state B (orange traces), 2^{nd} oxidized state B (brown traces). Bare substrates were used for the baseline (black traces).

FIG. 11: Spectroelectrochemical (SEC) performance of a laminated multi-state electrochromic device based on [MA1·4|FTO/glass] (active area: 1.7 cm×1.4 cm) as the working electrode and [PEDOT:PSS|FTO/glass] as the counter electrode (CE) in gel-electrolyte (LiClO₄/PMMA/ACN). (FIG. 11A-11B) Schematic representation of the multi-ECD of MA1·4. Photographs of all three states: state A, state B and state C of MA1·4. Reversible absorption spectral changes during spectroelectrochemical measurements corresponding to three redox states A, B and C of multi-electrochromic MA1·4 device. Bare substrates were used for the baseline (black). (FIG. 11C-11D) Gradual absorption spectral changes during spectroelectrochemical measurements. (FIG. 11E) Reversible transmittance spectral changes during spectroelectrochemical measurements. (FIG. 11F) Contrast ratios (ΔT, %) of MA1·2 at λ=480 nm, λ=530 nm and λ=590 nm, using double potential steps: −1.8 V to +3.0 V (state A and C) with pulse width of 20 s and SEC stability of multi-ECD using double potential steps: (i) −1.8 V to +3.0 V (state A to C) and (ii) −1.8 V to +2.0 V (state A to B) at λ=530 nm (Top to bottom). (FIG. 11G) Spectroelectrochemical switching in State A-C, A-B and A-C at λ=530 nm. (H) Spectroelectrochemical (SEC) switching at various pulse width between state A-C. (FIG. 11I) SEC stability of multi-ECD using double potential steps: (i) −1.8 V to +3.0 V (state A to C) and (ii) −1.8 V to +2.0 V (state A to B) at λ=530 nm. (J) Chronoamperometry of MA1·4-based device for switching in (i) state A-C and, (ii) state A-B. (FIG. 11K) Decay of the transmittance of an MA1·4-based device under an open-circuit potential from state C and B. Inset: Logarithmic plot showing a linear fit (R²=0.99).

FIGS. 12A-12B: Spectroelectrochemical (SEC) performance of a laminated multi-electrochromic device based on [MA2·4|FTO/glass] and [MA2·3|FTO /glass] as the working electrode and [PEDOT:PSS|FTO/glass] as the counter electrode (CE) in gel-electrolyte (LiClO₄/PMMA/ACN). (FIG. 12A) Photographs of all three states of [MA2·4|FTO/glass]: state A (blue color), state B (gray color) and state C (colorless). (FIG. 12B) Reversible absorption spectral changes during spectroelectrochemical measurements corresponding to the state A (blue top peak traces), state B (gray middle peak traces), and state C (brown lower traces) of a multi-electrochromic [MA2·4|FTO/glass]. Bare substrates were used for the baseline (black). (FIG. 12C) SEC stability of MA2·4 multi-ECD using double potential steps: (i) −1.8 V to +2.8 V (state A to C) and (ii) −1.8 V to +2.0 V (state A to B) at λ=585 nm (top to bottom). (FIG. 12D) Photographs of all three states of [MA2·3|FTO/glass]: state A (red color), state B (orange color) and state C (colorless). (FIG. 12E) Reversible absorption spectral changes during spectroelectrochemical measurements corresponding to the state A (red traces), state B (orange traces), and state C (brown traces) of a multi-electrochromic [MA2·3|FTO /glass]. Bare substrates were used for the baseline (black). (FIG. 12F) SEC stability of MA2·3 multi-ECD using double potential steps: (i) −2 V to +3.6 V (state A to C) and (ii) −2 V to +2.4 V (state A to B) at λ=566 nm (top to bottom).

FIGS. 13A-13L: Characterization and spectroelectrochemical (SEC) performance of [MA1·4|FTO/glass, resistance 10Ω/□] prepared by spray-coating. (FIG. 13A) AFM topography image, scans were made in AC mode using a silicon probe (Olympus Co. AC240). (FIG. 13B) SEM topography image. (FIG. 13C) SEM image showing the cross-section of [MA|FTO/glass 10Ω/□] that was milled by a 30 keV Ga⁺ focused ion beam (FIB). A Pt coating was used to prevent ion beam damage. (FIG. 13D) Normalized X-ray photoelectron spectroscopy (XPS) spectra showing the Os²⁺ 4f Fe²⁺ 2p, N 1 s, and Pd²⁺ 3d regions. (FIG. 13E) Gradual oxidation spectral changes while changing potential from 0.2-1.8 V. (FIG. 13F) Gradual reduction spectral changes while changing potential from 1.8-0.2 V. (FIG. 13G) Dependence of the contrast ratio (ΔT) at different λ=480 nm, 530 nm and 591 nm, using double potential steps: 0.2-1.8 V (switching between state A and C). (FIG. 13H) SEC while switching between different state at λ=530 nm, using double potential steps for (i) state A to C: 0.2-1.8 V, (ii) state A to B: 0.2-0.8 V and (iii) state B to C: 0.65-1.8 V. (FIG. 13I) SEC measurements of (2 cm×1 cm) MA1·4 using double potential steps: 0.4-1.8 V at different switching times in 0.1 M TBAPF₆/ACN electrolyte. (FIG. 13J) SEC switching using double potential steps for (i) 0.2-1.8 V and (ii) 0.2-0.8 V at λ=530 nm. nm. (FIG. 13K) SEC stability of MA1·4 using double potential steps: (i) 0.2-1.8 V and (ii) 0.2-0.8 V at λ=530 nm. (FIG. 13L) Chronoamperometry of MA1·4 using double potential steps: for (i) 0.2-0.8 V and (ii) 0.2-0.8V.

FIGS. 14A-14H: Formation, characterization and spectroelectrochemical (SEC) performance of [MA2·4|FTO/glass 10Ω/□] prepared by spray-coating. FIG. 14A Formation of MA2·4 on FTO/glass by spray coating the solution of complexes 2·4. (FIG. 14B) AFM topography image, scans were made in AC mode using a silicon probe (Olympus Co. AC240). (FIG. 14C) SEM topography image. (FIG. 14D) SEM image showing the cross-section of [MA2·4|FTO/glass, resistance 10Ω/□] that was milled by a 30 keV Ga⁺ focused ion beam (FIB). A Pt coating was used to prevent ion beam damage. FIG. 14E Normalized X-ray photoelectron spectroscopy (XPS) spectra showing the Fe²⁺ 2p, N 1 s, and Pd²⁺ 3d regions. (FIG. 14F) Gradual oxidation spectral changes while changing potential from 0.2-1.8 V. (FIG. 14G) SEC while switching between different state at λ=565 nm, using double potential steps for (i) state A to C: 0.2-1.8 V, (ii) state A to B: 0.2-1.05 V and (iii) state B to C: 0.95-1.8 V. (FIG. 14H) SEC switching using double potential steps for (i) 0.2-1.8 V and (ii) 0.2-0.8 V at λ=565 nm. (FIG. 14I) SEC stability of MA2·4 using double potential steps: (i) 0.2 -1.8 V and (ii) 0.2-1.05 V at λ=565 nm. (FIG. 14J) Chronoamperometry of MA1·4 using double potential steps: for (i) 0.2-0.8 V and (ii) 0.2-1.05 V.

FIGS. 15A-15E: Characterization and spectroelectrochemical (SEC) performance of [MA2·3|FTO /glass 10Ω/□] prepared by spray-coating. (FIG. 15A) AFM topography image, scans were made in AC mode using a silicon probe (Olympus Co. AC240). (FIG. 15B) SEM topography image. (FIG. 15C) SEM image showing the cross-section of [MA|FTO/glass 10Ω/□] that was milled by a 30 keV Ga⁺ focused ion beam (FIB). A Pt coating was used to prevent ion beam damage. (FIG. 15D) Gradual oxidation spectral changes while changing potential from 0.2-1.8 V. (FIG. 15E) SEC switching using double potential steps for (i) 0.2-1.8 V at λ=566 nm.

FIGS. 16A-16E: Spectroelectrochemical (SEC) switching of a laminated multi-electrochromic [MA1·4|FTO/glass] film (2 cm×2 cm) in different sates using [PEDOT:PSS|FTO/glass] as the counter electrode (CE) in gel-electrolyte (LiClO₄/PMMA/ACN). (FIG. 16A) SEC measurements using double potential steps: (i) state A to B: −1.8 V to +2.0 V and (ii) state A to C: −1.8 V to +3.0 V at λ=530 nm (FIG. 16B) SEC measurements using double potential steps: (i) state A to C: −1.8 V to +3.0 V and (ii) state B to C: −0.8 V to +3.0 V at λ=530 nm. (FIG. 16C) SEC measurements using double potential steps: (i) state A to B: −1.8 V to +2.0 V, (ii) state B to C: −0.8 V to +3.0 V and (iii) state A to C: −1.8 V to +3.0 V at λ=530 nm. (FIG. 16D) SEC stability measurements using double potential steps: (i) state A to C: −1.8 V to +3.0 V and (ii) state B to C: −0.8 V to +3.0 V at λ=530 nm. (FIG. 16E) Transmission spectral changes corresponding to state A and state C.

FIG. 17 Cyclic voltammograms (CVs) with scan rates of 0.05-0.9 V/s (left column), and exponential and linear correlations between the peak current vs scan rate and square root of the scan rate, during oxidation and reduction process (R²>0.99 for all fits) in a 0.1 M TBAPF₆/ACN electrolyte solution (second to left column), and scanning electron microscopy (SEM) images of surfaces (second to right column): [MA1|FTO/glass] film (A, B, C), [MA4|FTO/glass] film (D, E, F), and [MA1·4|FTO/glass] film (G, H, I). Comparison of XPS graph of NiCl₂ trapped MA1, MA4 and MA1·4 on FTO/glass (J,K). Comparison of region 850 ev-880 ev of [MA1·4|FTO/glass] films with and without NiCl₂ (L).

FIG. 18 is a scheme showing MA1, MA4 and a multi-component MA1·4 with their respective colors.

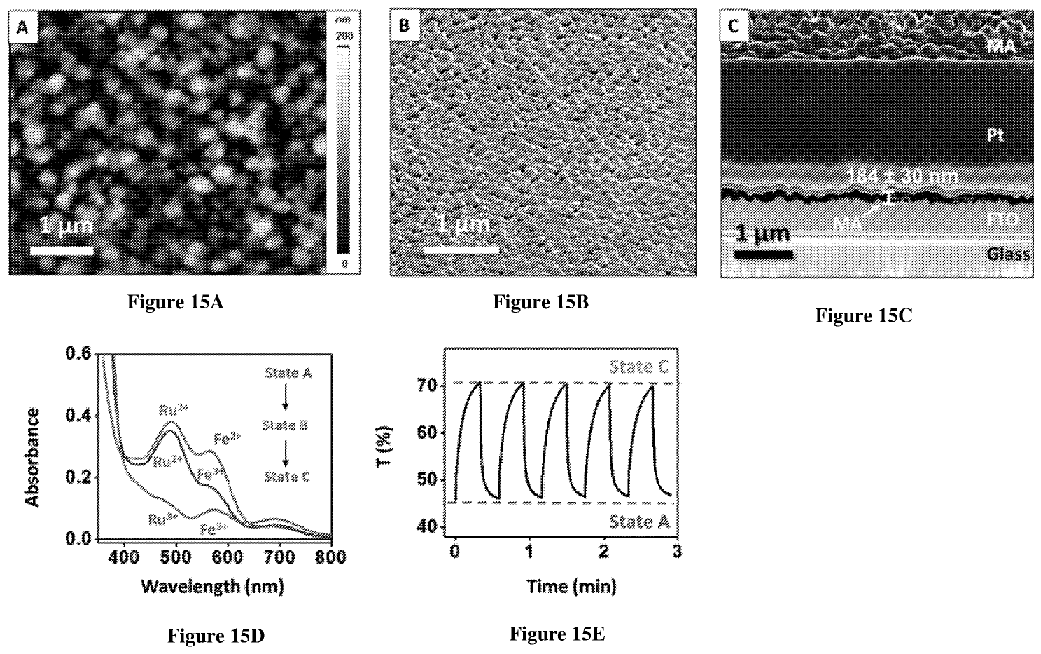

FIG. 19 Comparison of contrast ratio vs pulse width of single component [MA1|FTO/glass] (FIG. 19A), [MA4|FTO/glass] (FIG. 19B) and multicomponent [MA1·4|FTO/glass] (FIG. 19C), films (2 cm×2 cm), in a 0.1 M TBAPF₆/ACN electrolyte solution.

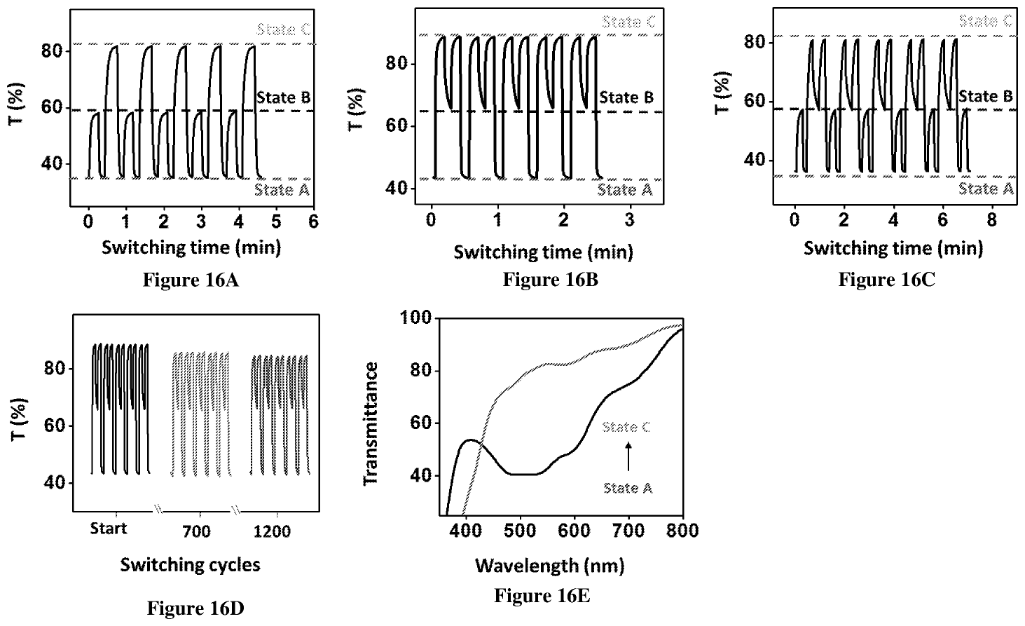

FIG. 20 (A) Comparison of X-ray photoelectron spectroscopy (XPS) spectras of [MAs|FTO/glass 10Ω/□] films after immersion in 10 mM solution of NiCl₂:6H₂O in ethanol. (A) [MA1|FTO/glass 10Ω/□], [MA4|FTO/glass 10Ω/□] and [MA1·4|FTO/glass 10Ω/□]. (B) [MA4|FTO/glass 10Ω/□], [MA2|FTO/glass 10Ω/□] and [MA4·2|FTO/glass 10Ω/□].

FIG. 21 Scheme of ion permeability based on electrochemical and NiCl₂ trapping study by X-ray photoelectron spectroscopy (XPS) spectra's of [MA4|FTO/glass 10Ω/□], [MA1|FTO/glass 10Ω/□] and [MA1·4|FTO/glass 10Ω/□] (A, B, C).

FIG. 22 Cyclic voltammograms (CVs) with scan rates of 0.05-0.9 V/s (left column, multiple graphs show different scan rates), and exponential and linear correlations between the peak current vs scan rate and square root of the scan rate (central column), during oxidation and reduction process (R²>0.99 for all fits) in a 0.1 M TBAPF₆/ACN electrolyte solution, and dependence of the contrast ratio (ΔT) on the switching time (right column). [MA2|FTO/glass] film (A, B, C), [MA4|FTO/glass] film (D, E, F), and [MA4·2|FTO/glass] film (G, H, I).

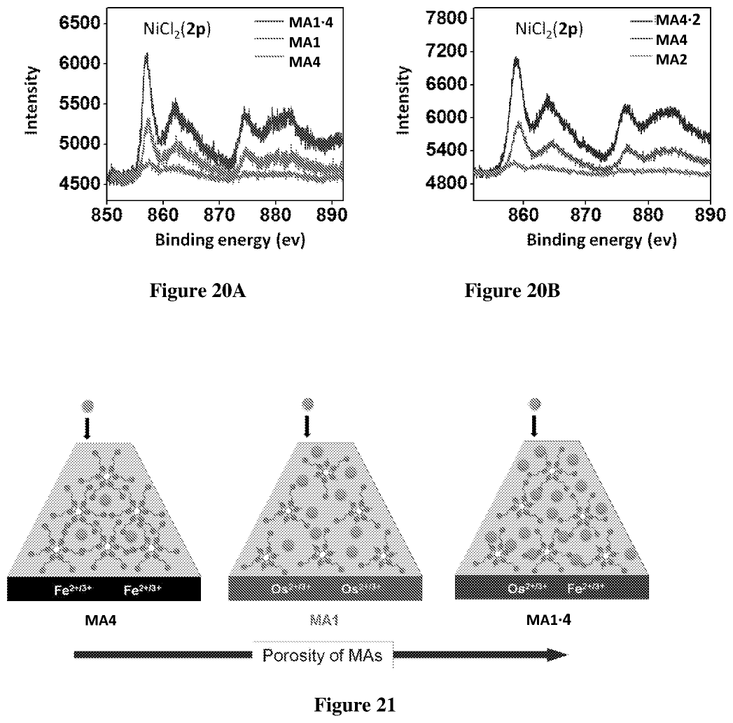

FIG. 23 Spectroelectrochemical (SEC) performance of a laminated multi-state electrochromic device based on [MA4·2|FTO/glass] (active area: 1.7 cm×1.4 cm) as the working electrode and [PEDOT:PSS|FTO/glass] as the counter electrode (CE) in gel-electrolyte (LiClO₄/PMMA/ACN). (A-B) Schematic representation of the multi-ECD of MA4·2. Photographs of all three states: state A, state B and state C of MA4·2. (C-D) Reversible absorption spectral changes during spectroelectrochemical measurements corresponding to three redox states A, B and C (C-D) Gradual absorption spectral changes during spectroelectrochemical measurements of multi-electrochromic MA4·2 device. Bare substrates were used for the baseline (black). (E) Reversible transmittance spectral changes during spectroelectrochemical measurements. (F) SEC switching of multi-ECD using double potential steps: (i) −1.5 V to +3.0 V (state A to C), (ii) −1.5 V to +2.0 V (state A to B) and (ii) −0.8 V to +2.0 V (state B to C) at λ=590 nm. (G) Spectroelectrochemical (SEC) switching at various pulse width between states A-C. (H) SEC stability of multi-ECD using double potential steps: (i) −1.5 V to +3.0 V (state A to C) and (ii) −1.5 V to +2.0 V (state A to B) at λ=590 nm.

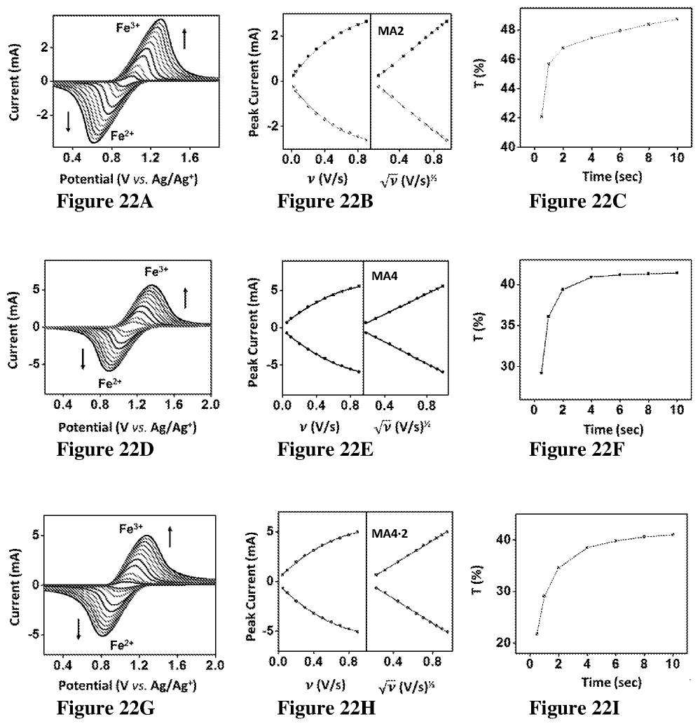

FIG. 24 Formation of metallo-organic assemblies (MAs). (A) Complexes used for the formation of the MAs. (B) Automated ultrasonic spray-coating of 0.2 mM (DCM/MeOH, 1:1 v/v) solutions of complexes 1 or 4, or 1·4, and 1.0 mM (THF) solution of PdCl₂(PhCN)₂ on transparent conducting oxides (TCOs). (MA4 and MA1·4 are not drawn). Complete experimental details are summarized in the examples below and in Table 3.

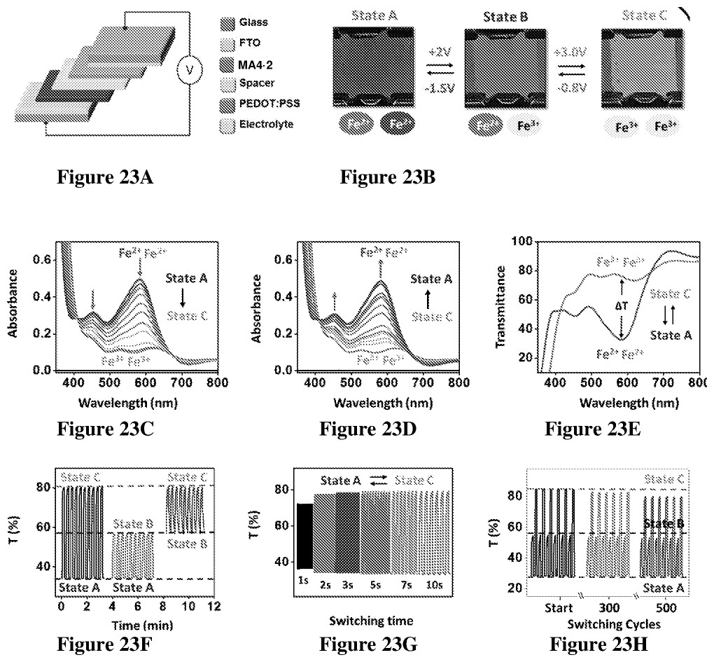

FIG. 25: In situ polymerization of liquid monomer electrolyte (HDODA, omnirad 184-resin, LiClO₄, PMMA, and ACN/PC) using UV-A light. (Step 1) Liquid monomer electrolyte drop-casted on the working electrode. (Step 2) Liquid monomer electrolyte sandwiched between working (glass/FTO//MA) and counter electrode (FTO/glass). (Step 3) Liquid monomer electrolyte was cured under UV-A light for 1 min to produce a solid electrolyte matrix, and (Step 4) Laminated electrochromic device connected to a potentiostat, based on [MA|FTO/glass] as the working electrode and [FTO/glass] as the counter electrode (CE) in solid polymer electrolyte (SPE).

FIG. 26 Electrochromic performance of [MA1//FTO/glass], and [MA4//FTO/glass] in a laminated device using a solid-state polymer electrolyte matrix. (A,B, left): schematic representation of the ECDs-based on MA1 and MA4. (A): photographs of the colored and bleached states (active area: 1.7 cm×1.3 cm) of MAL (B, top): photographs of the colored and bleached states (active area: 1.7 cm×1.3 cm) of MA4 without heating. (B, middle) Photographs of the fabricated electrochromic devices (MA4//FTO/glass) for continue cycling after heating at 60° C. for 24 h. (B, bottom) Photographs of the fabricated electrochromic devices (MA4//FTO/glass) for continue cycling after heating at 100° C. for 24 h.

FIG. 27 Electrochromic performance of MA1·4 on FTO/glass in laminated set-up using solid state polymer matrix. (A) Schematic representation of laminated device with [MA1·4|FTO/glass] film as working electrode. (B) Schematic representation of the three-states of multi-component [MA1·4|FTO/glass] film: state A (red color), state B (gray color) and state C (colorless color), using: HDODA, omnirad184-Resin, LiClO₄, PMMA, and ACN/PC as solid-state polymer electrolyte, with FTO/glass as counter electrode. (C) Photographs of the three states of laminated display (active area: 1.8 cm×1.3 cm) of MA1·4 display. (D) Chronoamperometric (CA) measurements using double potential steps when switching from A to B to C states: (i) −2 V to +1.8 V and (ii) +1.8 V to +2.8 V. Potential applied when switching from state C to B to A: (i) +2.8 V to −0.8 V and (ii) −0.8 V to −2 V.

FIG. 28 Spray coating process and characterization of [MA1|FTO/glass 10Ω/□] prepared by spray-coating. (B) SEM topography image. (C) SEM image showing the cross-section of [MA1|FTO/glass 10Ω/□] that was milled by a 30 keV Ga⁺ focused ion beam (FIB). A Pt coating was used to prevent ion beam damage. MA4 and MA1·4 are not shown.

FIG. 29 Optical images of the electrolyte precursor before (left) and after UV curing (right).

FIG. 30 Schematic diagram for UV cured polymerization of UV active gel electrolyte sandwiched between two FTO/glass substrates. (Step 1) Liquid monomer electrolyte was drop casted on FTO/glass substrate. (Step 2) liquid monomer electrolyte was sandwiched between two FTO/glass substrates (photograph of the device w/o molecular assemblies shown before UV curing the liquid electrolyte). (Step 3) liquid electrolyte was cured under UV light to produce a solid electrolyte matrix (photograph of the device w/o molecular assemblies shown after UV cured the liquid electrolyte).

FIG. 31 Photographs of the colored and bleached states (active area: 1.7 cm×1.3 cm) of spray coated [MA4|FTO/glass] without heating and after heating at 60° C. (top) and 100° C. (bottom).

FIG. 32 Embodiments of devices, systems and apparatuses of the invention with illustration of various optional elements/components.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

In one embodiment, this invention describes multi-color electrochromic behavior of molecular assemblies. In one embodiment, the molecular assemblies comprise two (or more) metal-organic compounds. The metal ion in one compound is different from the metal ion in the other compound. A mixture of the two compounds is applied to a transparent surface to form a layer. The metal ion is chosen such that it has two different chromic states. One color for the oxidized state and another color for the reduced state. The combination of two metal ions with two different color states for each ion, results in a layer where three chromic states are achievable as follows:
State 1: both ions are reduced;
State 2: one ion is oxidized, and the other ion is reduced;
State 3: both ions are oxidized.

Each state exhibits a different color, the color is derived from the combined absorption characteristics of the two ions at that certain sate.

Since the oxidation/reduction potential for the two ions is different, it is possible to reach a state where one ion is reduced while the other ion is oxidized.

The electrochromic state of the layer is thus controlled by the electrochemical potential applied to the layer.

For example, for a device comprising a layer of organic compounds comprising Fe- and Os-ions, three colors are obtained when the electrochemical potential applied to the device is varied:
State A (reduced Os, reduced Fe) red color;
State B (oxidized Os, reduced Fe) gray color;
State C (oxidized Os, oxidized Fe) colorless.

It is thus demonstrated in one embodiment, that devices showing multi-color electrochromic behaviors can be formed. The devices comprise coordination-based molecular assemblies. The devices exhibit three distinct redox states, states that are clearly visible to the eye and can be switched from one state to another upon the application of a potential. The devices presented herein provide superior durability/stability to the color change (e.g. at least 1200 color-change cycles or at least 2000 color change cycles depending on device features).

In other embodiments, this invention provides two-color electrochromic behavior of molecular assemblies. These molecular assemblies comprise one metal-organic compound in one embodiment. The metal-organic compound is applied to a transparent surface to form a layer. The metal ion is chosen such that it has two different chromic states. One color for the oxidized state and another color for the reduced state. Having one metal ion with two different color states, results in a layer where two chromic states are achievable as follows:
State 1: reduced;
State 2: oxidized;

Each state exhibits a different color. The electrochromic state of the layer is thus controlled by the electrochemical potential applied to the layer.

Process of Producing

In one embodiment, this invention provides a method of preparation of an electrochromic device, said method comprising:
a. providing a substrate;
b. applying a linker comprising a metal ion to said substrate by spray-coating, thus forming a linker layer on said substrate;
c. applying a metal-coordinated organic complex to said linker layer by spray coating, thus forming a layer of metal-coordinated organic complex on said linker layer;
d. optionally repeating steps b and c;
   thereby forming an electrochromic device comprising a substrate and comprising at least one layer of a linker and at least one layer of a metal-coordinated organic complex.

In one embodiment, the metal-coordinated organic complex comprises at least one functional group, said functional group capable of binding to the metal ion in the linker. In one embodiment, the functional group comprises a nitrogen atom.

In one embodiment, the binding comprises a coordination bond between said functional group and the metal ion of the linker.

In one embodiment, the metal-coordinated organic complex is polypyridyl complex.

In one embodiment, the spray coating steps for applying the metal linker and the organic complex are conducted at atomization pressure ranging between 0.75 kPa and 1.50 kPa and at a nozzle to substrate distance ranging between 3.0 and 8.0 cm, and at a spraying solution flow rate ranging between 0.4 and 0.8 mL/min and at room temperature.

In one embodiment, the spray coating steps for applying the metal linker and the organic complex are conducted at atomization pressure ranging between 0.75 kPa and 1.50 kPa. In one embodiment, the spray coating steps for applying the metal linker and the organic complex are conducted at a nozzle to substrate distance ranging between 3.0 and 8.0 cm. In one embodiment, the spray coating steps for applying the metal linker and the organic complex are conducted at a spraying solution flow rate ranging between 0.4 and 0.8 mL/min. In one embodiment, the spray coating steps for applying the metal linker and the organic complex are conducted at room temperature.

Other spraying parameters and other combinations of spraying parameters are possible and are compatible with embodiments of this invention as known to a person of ordinary skill in the art. Spraying parameters can be modified to fit a certain spraying apparatus. Different spraying apparatuses can be used in embodiments of this invention. Spraying parameters can be modified to fit certain spraying solution contents and spraying solution concentrations.

In one embodiment, the number of passes (spray passes) ranges between 1 and 5 or between 1 and 10 or between 2 and 7 or between 1 and 20. “Pass” means a spray event. For example, 3 spray passes refer to a substrate that was sprayed 3 consecutive times with a solution of a certain compound.

Each complete spray-deposition of a linker and a complex provides one deposition cycle. Repetition means how many deposition cycles have been performed. For example, 3 repetitions mean 3 layers of (linker+complex).

In one embodiment, the number of repetitions ranges between 1 and 5 or between 1 and 10 or between 2 and 7 or between 1 and 20 or between 1 and 100 or between 1 and 1000 or between 1 and 10,000. Any number of repetitions is possible in embodiments of this invention.

In one embodiment, the spraying is conducted such that the spraying nozzle is moved parallel to the substrate in a pattern along the X-Y substrate directions at a speed ranging between 3 and 7 mm/s.

The pattern of a pass can also be modified as required (e.g. left-right, zigzag, circular, oval, spiral) or any other pattern that will cover the surface in an efficient manner. Nozzle speed can also be changed according to some embodiments.

In some embodiments, the nozzle is moved, and the substrate is stationary. In another embodiment, the nozzle is stationary, and the substrate is moved.

In one embodiment, following application of the linker layer, following application of the metal-coordinated organic complex layer or a combination thereof, a washing step is conducted for washing the linker layer, for washing the complex layer or a combination thereof.

In one embodiment, following application of the linker layer, following application of the metal-coordinated organic complex layer or a combination thereof, a drying step is conducted for drying the linker layer, for drying the complex layer or a combination thereof.

In one embodiment, the washing solvent is selected from the group consisting of alcohols, ethers, esters, halogenated solvents, hydrocarbons, ketones, or a mixture thereof.

In one embodiment, both applying steps (linker and complex) are repeated to obtain from 2 to 80 linker/organic-complex layers.

In one embodiment, the metal ion in the linker is selected from the group consisting of Pd, Zn, Os, Ru, Fe, Pt, Ni, Ir, Rh, Co, Cu, Re, Tc, Mn, V, Nb, Ta, Hf, Zr, Cr, Mo, W, Ti, Sc, Ag, Au, and Y.

In one embodiment, the metal ion in the linker is different from the metal ion in the metal-coordinated organic complex.

In one embodiment, the polypyridyl complex is represented by Formula I:

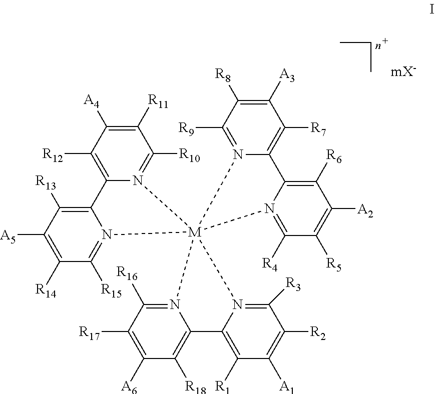

wherein
M is a transition metal selected from Fe, Os, Ru, Co, Ni, Mn, Cu, Zn, Ti, C, Cr, Rh, or Ir;
n is the formal oxidation state of the transition metal, wherein n is 0-6;
X is a counter ion;
m is a number ranging from 0 to 6;
R₁ to R₁₈ each independently is selected from H, halogen, —OH, —N₃, —NO₂, —CN, —N(R₂₀)₂, —CON(R₂₀)₂, —COOR₂₀, —SR₂₀, —SO₃H, —CH═CH-pyridyl, —(C₁-C₁₀)alkyl, —(C₂-C₁₀)alkenyl, —(C₂-C₁₀)alkynyl, —(C₁-C₁₀)alkoxy, cycloalkyl, heterocycloalkyl, aryl, heteroaryl, wherein the (C₁-C₁₀)alkyl, (C₂-C₁₀)alkenyl, (C₂-C₁₀)alkynyl, cycloalkyl, heterocycloalkyl, aryl, or heteroaryl may optionally be substituted with halogen, —OR₂₀, —COR₂₀, —COOR₂₀, —OCOOR₂₀, —OCON(R₂₀)₂, —(C₁-C₈)alkylene-COOR₂₀, —CN, —N(R₂₀)₂, —NO₂, —SR₂₀, —(C₁-C₈)alkyl, —O—(C₁-C₈)alkyl, —CON(R₂₀)₂, or —SO₃H;
A₁ to A6 each independently is a group of Formula III, i.e., a pyridine or pyridine derivative moiety, or of Formula IV, i.e., pyrimidine or pyrimidine derivative moiety, linked to the ring structure of the complex of general Formula I via R₁₉

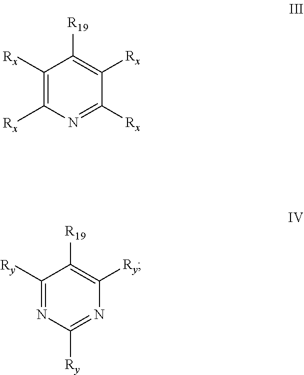

R₁₉ each independently is selected from a covalent bond, H₂C—CH₂, HC═CH, C≡C, N═N, HC═N, N═CH, H₂C—NH, HN—CH₂, —COO—, —CONH—, —CON(OH)—, —NR₂₀—, —Si(R₂₀)₂—, an alkylene optionally interrupted by one or more heteroatoms selected from O, S, or N, phenylene, biphenylene, a peptide moiety consisting of 3 to 5 amino acid residues,

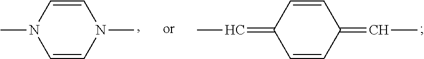

Rₓ and R_{y} each independently is selected from H, halogen, —OH, —N₃, —NO₂, —CN, —N(R₂₀)₂, —CON(R₂₀)₂, —COOR₂₀, —SR₂₀, —SO₃H, —CH═CH-pyridyl, —(C₁-C₁₀)alkyl, —(C₂-C₁₀)alkenyl, —(C₂-C₁₀)alkynyl, —(C₁-C₁₀)alkoxy, cycloalkyl, heterocycloalkyl, aryl, heteroaryl, protected carboxyl, or protected amino, wherein the (C₁-C₁₀)alkyl, (C₂-C₁₀)alkenyl, (C₂-C₁₀)alkynyl, cycloalkyl, heterocycloalkyl, aryl, or heteroaryl may optionally be substituted with halogen, —OR₂₀, —COR₂₀, —COOR₂₀, —OCOOR₂₀, —OCON(R₂₀)₂, —(C₁-C₈)alkylene-COOR₂₀, —CN, —N(R₂₀)₂, —NO₂, —SR₂₀, —(C₁-C₈)alkyl, —O-(C₁-C₈)alkyl, —CON(R₂₀)₂, or —SO₃H; and
R₂₀ each independently is H, (C₁-C₆)alkyl, or aryl.

In one embodiment, the polypyridyl complex is represented by Formula II:

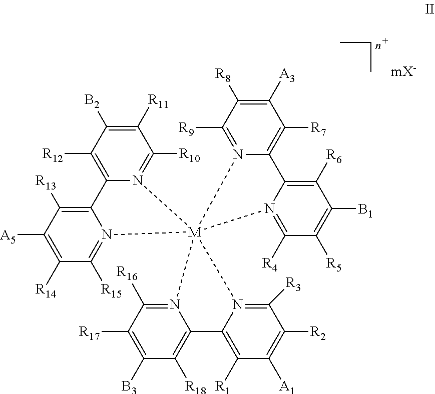

wherein
M is a transition metal selected from Fe, Os, Ru, Co, Ni, Mn, Cu, Zn, Ti, C, Cr, Rh, or Ir;
n is the formal oxidation state of M, wherein n is 0-6;
X is a counter ion;
m is a number ranging from 0 to 6;
R₁ to R₁₈ each independently is selected from H, halogen, —OH, —N₃, —NO₂, —CN, —N(R₂₀)₂, —CON(R₂₀)₂, —COOR₂₀, —SR₂₀, —SO₃H, —CH═CH-pyridyl, —(C₁-C₁₀)alkyl, —(C₂-C₁₀)alkenyl, —(C₂-C₁₀)alkynyl, —(C₁-C₁₀)alkoxy, cycloalkyl, heterocycloalkyl, aryl, heteroaryl, wherein the (C₁-C₁₀)alkyl, (C₂-C₁₀)alkenyl, (C₂-C₁₀)alkynyl, cycloalkyl, heterocycloalkyl, aryl, or heteroaryl may optionally be substituted with halogen, —OR₂₀, —COR₂₀, —COOR₂₀, —OCOOR₂₀, —OCON(R₂₀)₂, —(C₁-C₈)alkylene-COOR₂₀, —CN, —N(R₂₀)₂, —NO₂, —SR₂₀, —(C₁-C₈)alkyl, —O-(C₁-C₈)alkyl, —CON(R₂₀)₂, or —SO₃H;
A₁, A₃, and A₅ each independently is a group of Formula III, i.e., a pyridine or pyridine derivative moiety, or of Formula IV, i.e., pyrimidine or pyrimidine derivative moiety, linked to the ring structure of the complex of general Formula II via R₁₉

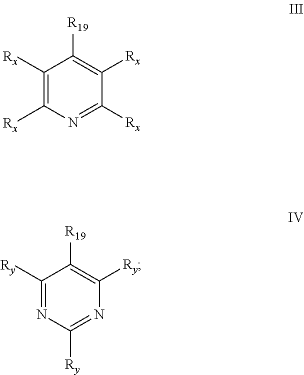

R₁₉ each independently is selected from a covalent bond, H₂C—CH₂, cis/trans HC═CH, C≡C, N═N, HC═N, N═CH, H₂C—NH, HN—CH₂, —COO—, —CONH—, —CON(OH)—, —NR₂₀—, —Si(R₂₀)₂—, an alkylene optionally interrupted by one or more heteroatoms selected from O, S, or N, phenylene, biphenylene, a peptide moiety consisting of 3 to 5 amino acid residues,

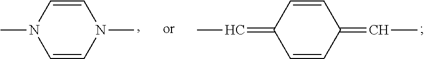

Rₓ and R_{y} each independently is selected from H, halogen, —OH, —N₃, —NO₂, —CN, —N(R₂₀)₂, —CON(R₂₀)₂, —COOR₂₀, —SR₂₀, —SO₃H, —CH═CH-pyridyl, —(C₁-C₁₀)alkyl, —(C₂-C₁₀)alkenyl, —(C₂-C₁₀)alkynyl, —(C₁-C₁₀)alkoxy, cycloalkyl, heterocycloalkyl, aryl, heteroaryl, protected carboxyl, or protected amino, wherein the (C₁-C₁₀)alkyl, (C₂-C₁₀)alkenyl, (C₂-C₁₀)alkynyl, cycloalkyl, heterocycloalkyl, aryl, or heteroaryl may optionally be substituted with halogen, —OR₂₀, —COR₂₀, —COOR₂₀, —OCOOR₂₀, —OCON(R₂₀)₂, —(C₁-C₈)alkylene-COOR₂₀, —CN, —N(R₂₀)₂, —NO₂, —SR₂₀, —(C₁-C₈)alkyl, —O-(C₁-C₈)alkyl, —CON(R₂₀)₂, or —SO₃H;

B₁ to B₃ each independently is selected from H, halogen, —OH, —N₃, —NO₂, —CN, —N(R₂₀)₂, —CON(R₂₀)₂, —COOR₂₀, —SR₂₀, —SO₃H, —CH═CH-pyridyl, —(C₁-C₁₀)alkyl, —(C₂-C₁₀)alkenyl, —(C₂-C₁₀)alkynyl, —(C₁-C₁₀)alkoxy, cycloalkyl, heterocycloalkyl, aryl, heteroaryl, protected carboxyl, or protected amino, wherein the (C₁-C₁₀)alkyl, (C₂-C₁₀)alkenyl, (C₂-C₁₀)alkynyl, cycloalkyl, heterocycloalkyl, aryl, or heteroaryl may optionally be substituted with halogen, —OR₂₀, —COR₂₀, —COOR₂₀, —OCOOR₂₀, —OCON(R₂₀)₂, —(C₁-C₈)alkylene-COOR₂₀, —CN, —N(R₂₀)₂, —NO₂, —SR₂₀, —(C₁-C₈)alkyl, —O-(C₁-C₈)alkyl, —CON(R₂₀)₂, or —SO₃H; and
R₂₀ each independently is H, (C₁-C6)alkyl, or aryl.

In one embodiment, the pyridyl complex is represented by one of the following formulas, or by a mixture of two or more of the following formulas, or by a combination of one or more of the following formulas with other pyridyl complexes:

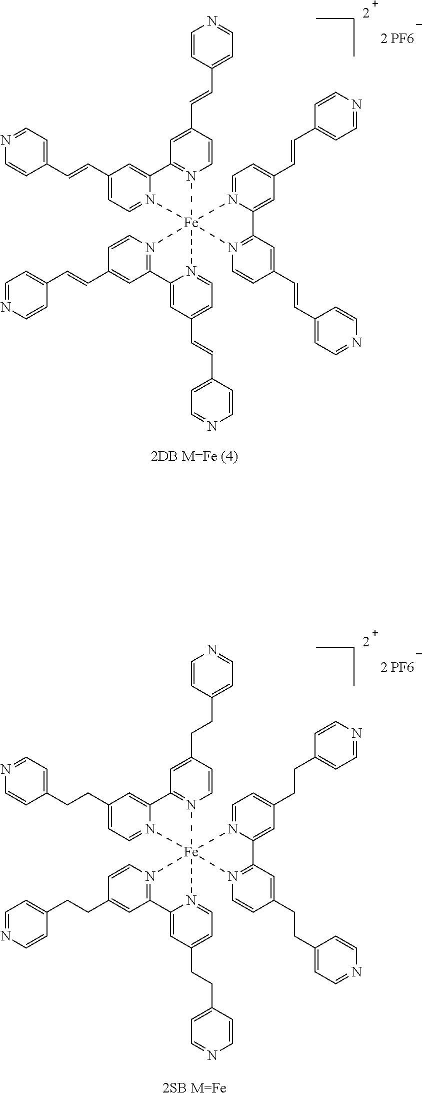

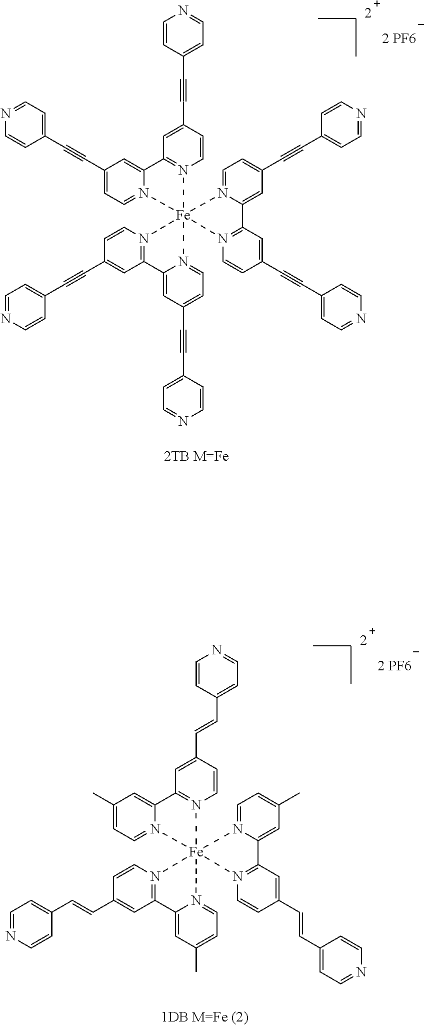

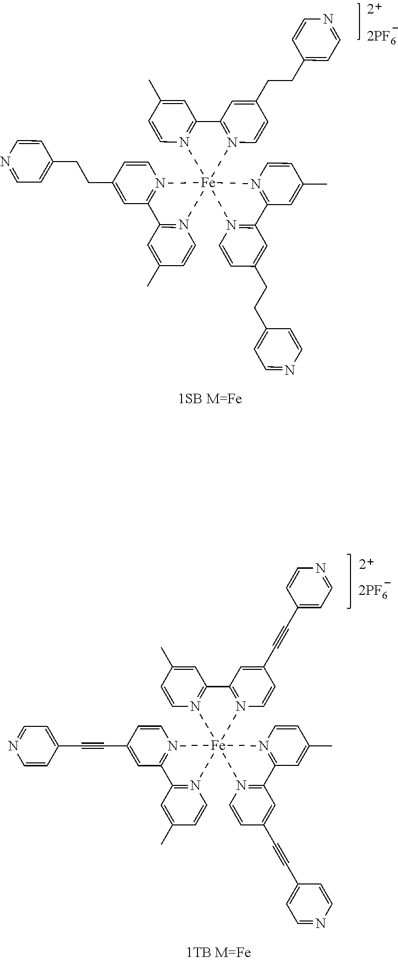

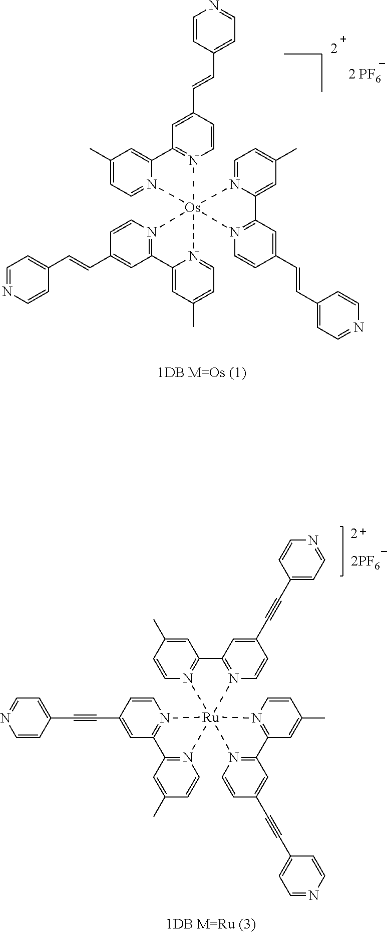

In one embodiment, the pyridyl complex used in embodiments of this invention and/or is present in devices of this invention is represented by one of the following formulas: (1), (2), (3) or (4) as noted herein above. In one embodiment, the pyridyl complexes used in embodiments of this invention and/or are present in devices of this invention are any mixture of two of these formulas: (1), (2), (3) and (4).

In one embodiment, the substrate or a portion thereof is conductive. In one embodiment, the substrate is selected from the group consisting of ITO, FTO, ITO or FTO-coated polyethylene terephthalate, ITO-coated glass or quartz, and FTO coated glass or quartz.

In one embodiment, the substrate or portion thereof is transparent in at least a portion of the UV range, in at least a portion of the visible range or in a combination thereof. In one embodiment, the substrate or portion thereof is transparent throughout the visible range. In one embodiment, the substrate is transparent in the wavelength range(s) wherein the metal ion(s) in the metal-coordinated organic complex (or the assemblies comprising the complexes), at a certain oxidation state, are not transparent. In one embodiment, the substrate is transparent or has more than 90% transmittance in the wavelength range(s) wherein the metal ion(s) in the metal-coordinated organic complex is/are not transparent at a certain oxidation/reduction state(s). In one embodiment, the substrate is transparent or has more than 90% transmittance in the wavelength range(s) wherein the molecular assembly comprising the metal ion(s) in the metal-coordinated organic complex has/have less than 10% or less than 20% transmittance at a certain oxidation/reduction state(s).

In one embodiment, the substrate transparency requirement is less strict, as long as the change in absorption spectrum of the assembly comprising the metal-coordinated organic complex upon oxidation/reduction, has enough contrast such that it can be viewed or detected even though the substrate is not completely transparent at a certain wavelength or at a certain wavelength range.

In one embodiment, the metal linker comprising a metal ion is a mixture of different linkers. In one embodiment, the polypyridyl complex is a mixture of two or more polypyridyl complexes.

It is to be noted that each layer comprising the metal-coordinated organic complex can include one type of complex in one embodiment, or more than one type of complex in another embodiment. Complex combination in a certain layer can be selected from but are not limited to:
two or more complexes with the same metal ions but with different ligands;
two or more complexes with different metal ions but with the same ligand;
two or more complexes with different metal ions and with different ligands.

For different layers in a multilayer assembly (i.e. an assembly comprising more than one deposition cycle), the different layers may comprise different combinations of complexes as exemplified herein above. In another embodiments, all the layers comprise the same combination of complexes.

In one embodiment, the step of applying a linker comprises applying the linker by spraying a solution comprising said linker, and wherein the step of applying at least one metal-coordinated organic complex comprises applying the metal-coordinated organic complex by spraying a solution comprising said metal-coordinated organic complex, and wherein said solutions comprise a solvent. In one embodiment, the solvent is selected from the group consisting of THF, alcohols, ethers, esters, halogenated solvents, hydrocarbons, ketones, or a mixture thereof In one embodiment, the solvent is selected from THF, CH₂Cl₂, MeOH or any combination thereof.

As described herein and in one embodiment, a device comprising one type of metal ion in the metal-coordinated organic complex (e.g. Fe ion) may exhibit two different states (oxidized/reduced), each characterized by a different color (by a different absorption spectrum). A device comprising two types of metal ions (e.g. Fe and Os) may exhibit three different states (oxidized/partially oxidized/reduced), each characterized by a different color (by a different absorption spectrum). Similarly, this may be extended to a device comprising three types of metal ions, and such device may exhibit four different states each characterized by a different color (by a different absorption spectrum). More than three different ions may be utilized in metal-coordinated organic complexes included in assemblies of this invention to generate multiple-color states for a device.

In one embodiment, the concentration of said linker in said solution and the concentration of said metal-coordinated organic complex in said solution ranges between 0.1 mM and 10 mM.

In one embodiment, the concentration of said linker in said solution and/or the concentration of said metal-coordinated organic complex in the solution used for spray-depositing is ranging between 1 mM to 50 mM, or between 1 mM and 12 mM, or between 1 mM and 100 mM, or between 0.1 mM and 100 mM, or between 1 mM and 10 mM, or between 10 mM and 40 mM, or between 0.01 mM and 10 mM, or between 0.001 mM and 500 mM. In one embodiment, the concentration of said linker in said solution and/or the concentration of said metal-coordinated organic complex in the solution used for spray-depositing is selected from 0.05 mM, 0.1 mM, 0.2 mM, 1 mM, 2, mM, 5 mM or any concentration in the range between these values.

In one embodiment, for mixtures of metal-coordinated organic complexes (e.g. a mixture of complex 1 and complex 4), the concentration of the two (or more) complexes is equi-molar. According to this aspect and in one embodiment, the concentration of each complex in a mixture is 1 mM, 2 mM or any concentration from the concentration list described herein above, such that the concentrations of two or more of the complexes in the mixture are equal. In one embodiment, the concentration of one complex in a mixture is different from the concentration of another complex in the mixture. According to this aspect and in one embodiment, the concentration of each complex in the mixture is chosen from the list of concentrations or from the list of ranges described herein above. In one embodiment, when more than two types of complexes are present in a mixture, any of the complexes types may have the same or different concentration as any of the other complex types present.

In one embodiment, the process of producing the molecular assembly by spray-coating is automated. According to this aspect and in one embodiment, a spraying system is provided, with two containers, one for the linker solution and one for the complex solution. The substrate is mounted at a predetermined nozzle-to substrate distance (e.g. 5.5 cm). The spraying process is automated such that the spraying of the linker and the spraying of the complex are conducted automatically. Switching between the two solutions for spraying of each is also automated. The spraying system is pre-programmed to follow the spray pattern (X-Y) and to spray according to predetermined atomization pressure, flow rate and other parameters as desired.

In one embodiment, the spraying device is an ultrasonic spraying device. In other embodiments, other spraying devices or systems are utilized.

In one embodiment, the process of forming the molecular assembly on a substrate is completed within an hour. In one embodiment, the time required to complete this process is ranging between 0.5 h and 1 h, between 0.5 h and 0.75 h, between 5 min and 30 min, between 5 min and 1 h, between 10 min and 30 min, between 1 min and 60 min. The number of double-layers (linker/complex) applied to the substrate affects the processing time in one embodiment.

Devices of this Invention

In one embodiment, this invention provides an electrochromic (EC) device, made by the method as described herein above.

In one embodiment, the thickness of the linker/organic layers measured perpendicular to the substrate surface ranges between 10 nm and 1 mm, or between 10 nm and 1000 nm or between 10 nm and 250 nm or between 50 nm and 250 nm or between 100 nm and 300 nm. In one embodiment, the thickness of the linker/organic layers measured perpendicular to the substrate surface ranges between 150 nm and 200 nm.

In one embodiment, the dimensions of the device parallel to the substrate surface comprise length and width ranging between 1 mm and 10 m. In one embodiment, the thickness of the device including the substrate, measured perpendicular to the substrate surface is ranging between 1 μm and 1 cm.

In one embodiment, the metal-coordinated organic complex comprises one type of metal ion. In one embodiment, the one type of metal ion comprise metal ion selected from Fe, Os, Ru, Co, Ni, Mn, Cu, Zn, Ti, C, Cr, Rh, or Ir. In one embodiment, the metal-coordinated organic complex comprises at least two types of metal ions. In one embodiment, the at least two types of metal ions comprise metal ions selected from Fe, Os, Ru, Co, Ni, Mn, Cu, Zn, Ti, C, Cr, Rh, or Ir. In one embodiment, the metal-coordinated organic complex is a polypyridine complex comprising two types of metal ions, said two types are Fe and Os ions or Fe and Ru ions or Ru and Os ions.

In one embodiment, the device has a contrast ratio between an oxidized and a reduced state of at least 10%, at least 20%, at least 30%, at least 40%, at least 50% or at least 60%, or a contrast ratio ranging between 10% and 20%, between 10% and 50%, between 25% and 50%, between 10% and 40%, between 10% and 70%.

In one embodiment, the device is able to retain at least 90% of its maximum contrast ratio after 1000 switching cycles between oxidized and reduced state(s). In one embodiment, the device is able to retain at least 90% of its maximum contrast ratio after 1500 switching cycles between oxidized and reduced state(s).

In one embodiment, stability of the devices is evidenced by the number of switching cycles that can be performed while keeping adequate contrast ratio. In one embodiment, the number of switching cycles for an operable device of this invention is higher than 1200 cycles, higher than 1500 cycles or higher than 2000 cycles. In one embodiment, the number of switching cycles for an operable device of this invention ranges between 1000 and 5000 cycles.

In one embodiment, devices of this invention have the ability to maintain the transmittance value (or maintain a lower yet operable transmittance value) after the applied potential is switched off and are therefore applicable for information storage. According to this aspect and in one embodiment, the measured decay time for a certain redox state when the applied potential is turned off is ˜25 min or ˜90 min. In one embodiment, decay time ranges between 1 min and 100 min. In one embodiment, decay time ranges between 25 min and 180 min.

In one embodiment, the device further comprising a power supply and electrical connections, said electrical connections connecting said device to the power supply wherein:
a first connection connecting said substrate to a first pole of said power supply;
a second connection connecting said metal-coordinated organic complex layer directly or through intermediate layers to a second pole of said power supply.

In one embodiment, the intermediate layers comprise an electrolyte, a storage layer, a spacer or any combination thereof.

In one embodiment, devices of this invention comprise:
a substrate;
a molecular assembly comprising:
a first double-layer, said double-layer comprising:
   a metal-ion linker layer; and
   a layer comprising metal-coordinated organic complex;
   wherein said linker layer is attached to said substrate and said metal-coordinated organic complex is attached to said linker layer;
optionally one or more additional double-layer(s) comprising:
a metal-ion linker layer; and
a layer comprising metal-coordinated organic complex;
   wherein said additional layer(s) are arranged on top of said first double-layer.

In one embodiment, devices of this invention further comprise:
a liquid electrolyte, a gel electrolyte or a solid electrolyte; and
an electrode;
   wherein said molecular assembly is in contact with said electrolyte and said electrolyte is in contact with said electrode.

In one embodiment, devices of this invention further comprise a charge storage layer (storage layer) placed between the electrode and the electrolyte. According to this aspect and in one embodiment, the electrolyte is in contact with the storage layer and not with the electrode. The storage layer is in contact with the electrode according to this embodiment.

In one embodiment devices of this invention further comprise a spacer, the spacer surrounds the electrolyte and separates the electrode from the molecular assembly. In one embodiment, the spacer contains and secures the electrolyte in the device.

In one embodiment, the molecular assembly is applied to one of the largest surfaces of the substrate. For example, for a flat rectangular substrate, the molecular assembly is applied to one of the two larger surfaces of the substrate in one embodiment.

In one embodiment, devices of this invention are capable of being switched between 2 or more chromic states (i.e. to exhibit two or more different absorption spectra). According to this aspect and in one embodiment, one chromic state is a colorless state. According to this aspect and in one embodiment, one chromic state is a transparent state in the visible range. In one embodiment, one chromic state is a transparent state in a wavelength range other than the visible range.

In one embodiment, this invention provides a smart window comprising the device as described herein above, wherein said substrate is transparent in the visible-light range and wherein the lateral length and width of said window measured parallel to the largest surface of said substrate is ranging between 1 cm to 10 m.

In one embodiment, this invention provides a switch comprising the device as described herein above. In one embodiment for the switch, the substrate is transparent in at least a portion of the visible-light range.

In one embodiment, this invention provides a memory device or an encoder comprising:
the device as described herein above, wherein said substrate is transparent in at least a portion of the visible-light range;
an optical detector.

In one embodiment, the substrate is not transparent in the visible-light range.

In one embodiment, the substrate material comprises a metal, a metal alloy, a metal oxide, silicon oxide or any combination thereof. In one embodiment, the substrate is selected form the group consisting of: silicon oxide, tin oxide, indium tin oxide. In one embodiment, the substrate is coated. In one embodiment, the substrate is electrically-conductive. In one embodiment, the substrate is non-electrically-conductive, and it is coated by a conductive layer. According to this aspect and in one embodiment, the substrate and the coating are referred to as “the substrate”. In other embodiments, the coating layer is referred to as “the substrate”.

In one embodiment, the optical detector comprises any optical detector known in the art. In one embodiment, the optical detector is or comprises a camera. In one embodiment, the optical detector is chosen or tuned for detecting a certain wavelength or a certain wavelength range.

In one embodiment, smart windows, switches, optical switches, memory devices, encoders and any other device of this invention further comprise optical elements such as filters, lenses, objectives, light source(s), gratings, optical fibers, prisms, etc.

In one embodiment, this invention provides a system comprising a device of this invention. In one embodiment, this invention provides an apparatus comprising a device of this invention. In one embodiment, devices, apparatuses and systems of this invention further comprise a computer, a display, electronic components, calculation algorithms, operation algorithms etc. In embodiments, devices, apparatuses and systems of this invention are operated manually, automatically, or using a combination of manual and automatic operation.

In one embodiment, this invention provides a display comprising a device of this invention. In one embodiment, at least one intermediate layer is present in the device between the MA and the connection to the power supply. In one embodiment, the intermediate layers comprise an electrolyte. In one embodiment, the electrolyte is a solid electrolyte. In one embodiment, the device comprises an electrode or electrodes. In one embodiment, the conducting surface of the substrate is one electrode and the MA is connected to another electrode. The two electrodes are connected to a power supply in one embodiment. In one embodiment, the display comprises multiple electrochromic devices such that each electrochromic device forms one or more pixel(s) in the display. According to this aspect and in one embodiment, the display can display an image, a pattern, written text, drawing, a code etc. According to this aspect and in one embodiment, the image, the pattern, the text or the code can be changed/appear/erased by changing the voltage applied to each pixel. In one embodiment, in order to display an image or a written text, the device itself is constructed in the shape of the specific image/text. According to this aspect and in one embodiment, the device shape is a pattern that matches an image/text/code. According to this aspect and in one embodiment, such pattern can be embedded in a background made of a different material, the background surrounds the device of this embodiment.

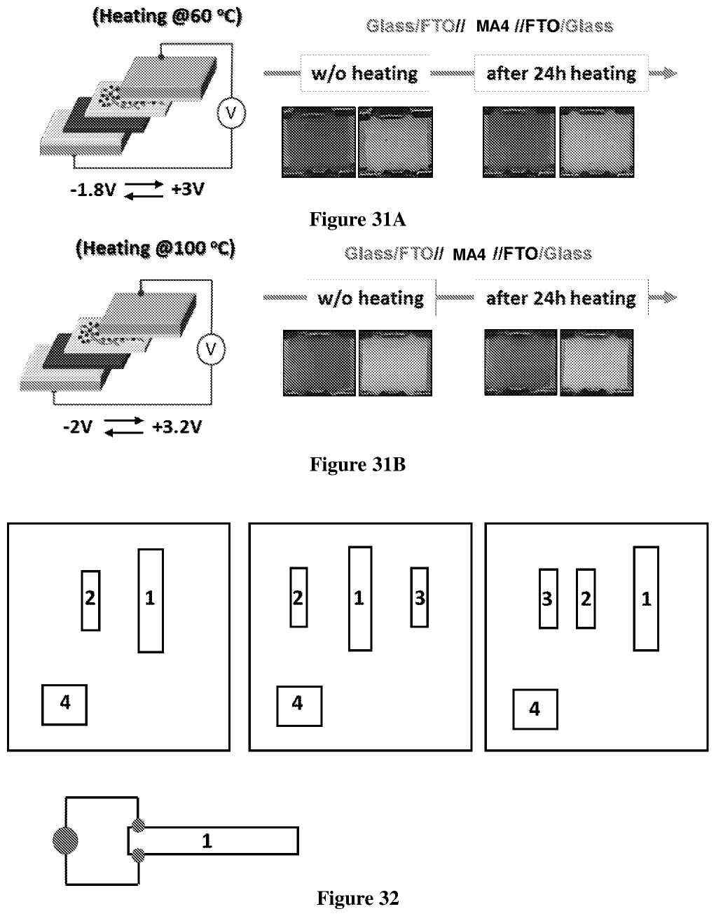

Illustrations of embodiments of devices, systems and apparatuses are shown in FIG. 32, wherein element 1 is or comprises the device comprising a substrate and the molecular assembly. Element 2 is an optional irradiation source, element 3 is an optional detector that can be placed on the side opposing the irradiation source or on the same side as the irradiation source for non-transparent or partially transparent electrolyte or for non-transparent or partially transparent substrates. Element 4 describe additional optional elements such as gauges, monitors, electronic components, optical components, mechanical components, optical fibers, wires and connectors, computer, processor, display, touch-screen, other user interfaces, knobs, switches etc. as described herein above and as known in the art. The configuration of the elements in the figure is an example. Other orientations, different distribution, various relative location of the elements, less or additional elements and different scales are included in this invention. The presence of elements 2, 3 and 4 or any combination thereof is optional. In some embodiment, the only element in devices of this invention is element 1 in FIG. 32. In one embodiment, a device of this invention comprises or consists of a device as described herein (a substrate and a molecular assembly attached thereto), and connections to a power supply (lines connecting device 1 to a large circle=power supply, see FIG. 32 bottom image). In one embodiment, the device comprises connections or inputs/outputs that can be connected to a power supply. In some embodiments, the device comprises the power supply. In some embodiments, the device does not comprise the power supply but can be connected to it when required.

In one embodiment, the irradiation source is a natural source, e.g. sun or sun light. In one embodiment, the irradiation source is a lamp, a laser, LED etc. In one embodiment, the irradiation source is included in the device/system and in other embodiments the irradiation source is not included in the device/system.

In one embodiment and as described herein, the device comprises solid electrolyte. In one embodiment, the solvent-free electrolyte used, improves the performance of the ECDs without the need for coating the counter electrode with any ion storage layer. According to this aspect and in one embodiment, the device comprises solid electrolyte and it does not comprise an additional ion storage layer. In other embodiments, the device comprises solid electrolyte and ion storage layer. In one embodiment, the description exemplified below for a display comprising solid electrolyte is applicable to other devices of this invention and is not limited to displays. For example, the device comprising solid electrolyte can be used as an optical switch, memory device, encoder etc.

In one embodiment, thickness ranges for the solid electrolyte layer are between 100-210 μm. In embodiments, thickness ranges for the solid electrolyte layer range between 50 μm to 500 μm. In embodiments, thickness ranges for the solid electrolyte layer range between 10 μm to 300 μm. Other thickness ranges are applicable to devices of this invention.

In one embodiment, the composition used to form the solid electrolyte layer is Polymethylmethacrylate (PMMA; 40 mg), 50 mg of LiClO₄, (325 μL) of UV active monomer 1,6-hexanediol diacrylate (HDODA), and 16 mg of photo-initiator omnirad-184. These materials were combined in a 1 mL propylene carbonate/acetonitrile (PC/ACN, 1:1) solution.

Other possible compositions include any combination of: Polymer: Polyethyleneglycol diacrylate (PEGDA) or polyoxypropylene glycol (PPG) or polydimethylsiloxane (PDMS); Monomer: Butyl acrylate (BA); Photo-initiator: 2,2-dimethoxy-2-phenyl-acetophenone (DMPAP); Salts: e.g. LiCF₃SO₃ or Li(CF₃SO₂)₂N or TBAPF₆ or NaClO₄.

Other materials/polymers/monomers/salts/initiators/compositions/solvents can be included/used in compositions used for the formation of the solid electrolyte as known in the art.

Additional properties of devices, systems and apparatuses of this invention are derived from their preparation method and are described in further detail in the device preparation section herein above.

Methods of Use of the Invention

In one embodiment, this invention provides a method of changing the absorption spectrum of the device as described herein above, the method comprising:
providing a device comprising:
   a substrate;
   a first linker layer, said layer attached to said substrate;
   a first metal-coordinated organic complex layer, said metal-coordinated organic complex comprising one type of metal ion, said complex layer is attached to said linker layer;
   optionally additional alternating layers of said linker and of said metal-coordinated organic complex constructed on top of said first metal-coordinated organic complex layer;
wherein said metal-coordinated organic complex is electrochromic such that when a certain voltage is applied to it, the oxidation state of said metal ion is changed and wherein said oxidation state change causes a change in the absorption spectrum of said metal-coordinated organic complex;
applying voltage to said device, thus changing the oxidation state of said metal ion, thereby inducing change in the absorption spectrum of said metal-coordinated organic complex, thus changing the absorption spectrum of said device.

In one embodiment, the substrate is at least partially transparent in the visible range.

In one embodiment, the voltage varies between (−3.0) V and 3.0 V. In one embodiment, the voltage applied to the device ranges between 0.0 V and 2 V, between 0.0 V and 1.8 V, between −1.2 V and 2.8 V, between −2 V and 2 V, between −1 V and 1 V, between −1 V and 2V, between 0.1 V and 2 V. Other voltage values and ranges are applicable to devices of this invention and are chosen in view of the oxidation/reduction properties of the metal ions used in the metal-coordinated organic complexes of this invention.

In one embodiment, the change in absorption spectrum is reversible.

In one embodiment, the method further comprising applying a second voltage to said device, thus changing the absorption spectrum of said device back to its initial spectrum (the spectrum prior to application of the first voltage).

In one embodiment, this invention provides a method of changing the absorption spectrum of the device as described herein above, the method comprising:
providing a device comprising:
   a substrate;
   a first linker layer, said layer attached to said substrate;
   a first metal-coordinated organic complex layer, said metal-coordinated organic complex comprising two types of metal ions, said complex layer is attached to said linker layer;
   optionally additional alternating layers of said linker and of said metal-coordinated organic complex constructed on top of said first metal-coordinated organic complex layer;
   wherein said metal-coordinated organic complex is electrochromic such that when a certain voltage is applied to it, the oxidation state of at least one type of said metal ions is changed and wherein said oxidation state change causes a change in the absorption spectrum of said metal-coordinated organic complex;
applying a first voltage to said device, thus changing the oxidation state of a first of said metal ions, thereby inducing change in the absorption spectrum of said metal-coordinated organic complex, thus changing the absorption spectrum of said device.
applying a second voltage to said device, thus changing the oxidation state of a second of said metal ions, thereby inducing an additional change in the absorption spectrum of said metal-coordinated organic complex, thus changing the absorption spectrum of said device.

In one embodiment, the substrate is at least partially transparent in the visible range. In one embodiment, the voltage varies between (−3.0) V and 2.0 V. In one embodiment, the change in ab sorption spectrum is reversible.

In one embodiment, the method further comprising applying a third voltage to said device, thus changing the absorption spectrum of said device back to its initial spectrum or back to its intermediate spectrum as described herein below. In one embodiment, the intermediate spectrum is the spectrum obtained after application of a first voltage to the device as described herein above.

In one embodiment, when a device or the molecular assembly in the device is brought back to an intermediate state or to an initial state (upon one or more voltage switching cycles), a small deviation from the initial/intermediate spectrum can occur due to incomplete transformation of the oxidation state, due to structure modifications etc. Such deviation in some embodiments, does not interfere with device operation/function. According to this aspect and in one embodiment, small changes in intensity at certain wavelengths of the spectrum are within the range of a certain “initial state”, a certain “intermediate state” or within the range of any other oxidation state of the device.

In one embodiment, this invention encompasses methods of spray-depositing multiple layers of electrochromic materials onto a substrate thereby creating a multilayered EC assembly. The invention also encompasses multilayered EC materials and devices composed of mixtures of one or at least two metal polypyridyl complexes. Not to be limited by theory, it is believed that the metal linker complexes to a polypyridyl compound thereby forming the bond between the layers. The layer-by-layer spray-coating technique described herein generates well-designed nanostructures. For example, and in one embodiment, it was shown that different layers constructed of Fe-polypyridyl-complex and Pd metal linker form a 3D coordination network with particular advantageous properties.

In one embodiment, methods of the invention produce EC material that is thermally and electrochemically robust in air with very high contrast ratios (ON/OFF ratios for some applications). The EC material operates under low voltage and it has practical switching times. Such EC material that has very high ON/OFF ratios, homogenous coating, low-voltage operations, high electrochemical stability and durability (such as light and thermal durability), color versatility, and short switching times, is useful in a variety of applications.

The multilayered EC material has unique electrical properties suitable in applications such as smart windows, electrochromic windows, smart mirrors, optical filters, frequency doubling devices, spatial light modulators, pulse shapers, displays, signs, plastic electronics, lenses, sensors, etc. Methods of this invention are used for the formation of electrochromic coatings. Methods of this invention are used for the formation of electrochromic films.

In some embodiments, EC materials of this invention are capable to retain high values of % ΔT, i.e., >90%, >95%, or >97%, after at least 1000, but preferably for more than 3,000, 5,000, or 10,000 electrochemical switching cycles when immersed in an electrolyte solution or exposed to electrolyte gel or solid electrolyte. The EC materials and devices of this invention are stable under exposure to air and to visible/UV light over a period of a few hours, a few days, months or years. In one embodiment, the EC material is able to retain high values of % ΔT, i.e., >80%, >90%, >95%, or >97% or >99%, after at least 1000, but preferably more than 3,000, 5,000, 10.000 or 100,000 electrochemical switching cycles when immersed in an electrolyte solution or being in contact with electrolyte gel or solid electrolyte and exposed to air, and/or to extreme atmosphere temperatures and to visible/UV light over a period of a few hours to a few years.

In one embodiment, the EC materials of this invention retained >90% of the original value of their contrast ratio after >1000 switching cycles.

In one embodiment, the substrate includes, but is not limited to, a material selected from glass, doped glass, ITO-coated glass, FTO-coated glass, silica, silicon, doped silicon, Si(100), Si(111), SiO₂, SiH, silicon carbide mirror, quartz, a metal, metal oxide, a mixture of metal and metal oxide, group IV elements, polydimethylsiloxane (PDMS) and related organic/inorganic polymers, mica, organic polymer, plastic, zeolite, a membrane, optical fiber, ceramic, metalized ceramic, alumina, electrically-conductive material, semiconductor. The organic polymer includes, but is not limited to, polyacrylamide, polystyrene, and polyethylene terephthalate. The substrate may be in the form of beads, microparticles, nanoparticles, quantum dots, nanotubes, films, flat flexible surfaces, or flat rigid surfaces. The substrate is at least partially optically transparent to visible, ultraviolet (UV), infrared (IR), near-IR (NIR), and/or other visible and non-visible spectral ranges. In one embodiment, the substrate is a rigid support comprising ITO- or FTO-coated glass or a flexible support of ITO coated PET. In one embodiment, the substrate is selected from the group consisting of ITO- or FTO-coated polyethylene terephthalate, ITO-coated glass or quartz, and FTO-coated glass or quartz. Optionally, the substrate comprises a template or coupling layer. In one embodiment, the substrate is a non-flat flexible substrate. In one embodiment, the substrate is a curved flexible substrate.

Preferably, the substrate is transparent in the visible range and has conducting properties. The substrate can be an n-type semiconductor with high carrier concentration, which leads to low electrical resistivity. High transmission in the visible and near-IR regions of the electromagnetic spectrum due to a wide band gap is also a desirable property of the substrate in some embodiments.

Metals used in linkers of this invention include those that can work as a metal linker between the substrate and the pyridyl compound or complex material or between two pyridyl compounds or complex materials. In the latter case, the pyridyl complex may be the same or different. Typical linker metals include, but are not limited to, transition metals, lanthanides, actinides, or main group elements. Transition metals include Zn, Os, Ru, Fe, Pt, Pd, Ni, Ir, Rh, Co, Cu, Re, Tc, Mn, V, Nb, Ta, Hf, Zr, Cr, Mo, W, Ti, Sc, Ag, Au, and Y. Lanthanides include La, Ce, Pr, Nd, Pm, Sm, Eu, Gd, Tb, Dy, Ho, Er, Tm, Yb, or Lu. Actinides include Ac, Th, Pa, U, Np, Pu, Am, Cm, Bk, Cf, Es, Fm, Md, No, or Lr. Main group elements include Zn, Ga, Ge, Al, Cd, In, Sn, Sb, Hg, Tl, or Pb. In one embodiment, the metal is Pd. The metal may be applied as a coordinate metal in either neutral or in an oxidation state. For instance, Pd can be applied as Pd or a Pd(II)-based complex. An example of Pd(II)-based complex is PdCl₂(PhCN)₂. Further, the linker metals or linker metal complexes are applied by spraying from solution. Suitable solution solvents include, but are not limited to, ethers such as tetrahydrofuran and ethyl ether. Metals in the metal-coordinated organic complexes of the invention can be any of the metals described herein above.

As used herein, and in one embodiment, the term “pyridyl complex” refers to a metal ion having one or more pyridyl compounds coordinated therewith. For example, the term “pyridyl complex” refers to a metal ion having two, three, or four pyridyl compounds coordinated therewith.

In one embodiment, the rinsing step is performed with at least one volatile organic solvent. Such volatile organic solvents include those capable of evaporating at room temperature. Typical volatile organic solvents include, but are not limited to, CH₂Cl₂, acetone, methanol, ethanol, THF, acetonitrile, among others.

Gasses suitable for use in the invention for the drying step, include, but are not limited to, nitrogen, argon, helium, neon, xenon, and radon. Preferably, the gas is nitrogen. Alternatively, the drying step can be air drying.

It is to be noted that in embodiments of this invention, rinsing the layers or drying the layers or any combination thereof can be applied to the layers after their formation such that:
one or more linker layer(s) are rinsed and/or dried, but the complex layers are not;
one or more complex layer(s) are rinsed and/or dried, but the linker layers are not;
none of the layers is rinsed/dried;
all linker layer(s) and all complex layer(s) are rinsed and/or dried.
rinsing and/or drying is performed only after all the linker/complex layers have been formed;

No rinsing/drying is applied prior to completion of all deposition cycles.

Any other rinsing and/or drying scheme can be employed in embodiments of this invention.

In one embodiment, no template or coupling layer is used or is present between the substrate and the metal linker layer in EC materials of this invention. According to this aspect and in one embodiment, the linker layer is applied directly to the substrate. In one embodiment, the layer-application steps are performed manually. In one embodiment, the layer-application steps are performed in a partially automated manner or in a fully automated manner as described herein above. Automation of the layer application technique results in fast fabrication of the EC materials in one embodiment.

Embodiments that are described herein for polypyridyl complexes are suitable for other metal-coordinated organic complexes as well. Embodiments that are described herein for Pd metal linkers are suitable for other metal linkers as well. Counter ions (the negative ion) in metal-coordinated organic complexes of this invention can be any counter ion as known to the skilled artisan. For example, the counter ion can be PF₆⁻, Cl⁻, Br⁻, I⁻, NO₃⁻. Any embodiment described herein with reference to a complex comprising PF6⁻ counter ion is compatible with the same complex having a different counter ion and is considered part of this invention. In one embodiment, the growth of the layers in assemblies of this invention is such that the thickness of each layer is the same or is similar to the thickness of other layers in the assembly. In other embodiments, various layer thicknesses can be obtained for different layers in an EC material of this invention.

In one embodiment, devices, systems or apparatuses of this invention further comprise a light source. In some embodiments, the light source is used to irradiate the device in order to determine transmittance at a certain wavelength or at a certain wavelength range. In one embodiment, the light source produces light at a certain wavelength or at a small wavelength range. In one embodiment, the light source produces light at a large wavelength range, for example at the complete visible range. In some embodiments, the light source may be accompanied by optical filters to adjust the irradiated light as needed.

Definitions

The terms ‘complex’, ‘organic complex’, ‘metal-organic complex’ are sometimes used interchangeably to replace the term ‘metal-coordinated organic complex’ for simplicity.

When providing a voltage range, e.g. between 0.0 V to 3.0 V, it is noted that voltage in this voltage range can be applied such that the positive pole is connected to a first electrode and the negative pole to the second electrode of a device of this invention, or vice-versa, i.e. the positive pole is connected to the second electrode and the negative pole to the first electrode of a device of this invention. Accordingly, the voltage range applied to devices of this invention in some embodiments, ranges between (−3)V and 3V. The conductive portion of the substrate functions as one of the electrodes in one embodiment.

Abbreviations disclosed include DCM for dichloromethane, TCO for transparent conductive oxides, ECD for electrochromic devices, FTO for fluorine-doped tin oxide, ITO for indium tin oxide, EC for electrochromic, MA for molecular assemblies.

In one embodiment, ‘transparent’ means transparent in the visible range. In other embodiments, ‘transparent’ means transparent to other wavelength ranges. In some embodiments, transparent means that light of a certain wavelength (visible or non-visible) is transferred through said material.

In embodiments where different colors (e.g. one color and another color) are discussed, it is to be noted that any one of the colors can be “transparent” or “colorless”. A ‘colorless’ or ‘transparent’ state is considered as a certain “color” for simplicity in some embodiments.

In embodiments, as described herein above, spray-deposition of the layers, device preparation or a combination thereof is conducted at room temperature. However, it is to be noted that methods of preparation as described herein can be performed at other temperatures, higher or lower than room temperature. Room temperature is usually around 18-25° C. but can be defined as any temperature between 20-30° C., 10-30° C., 0-40° C., (−10)-40° C. or (−20)-50° C., etc.

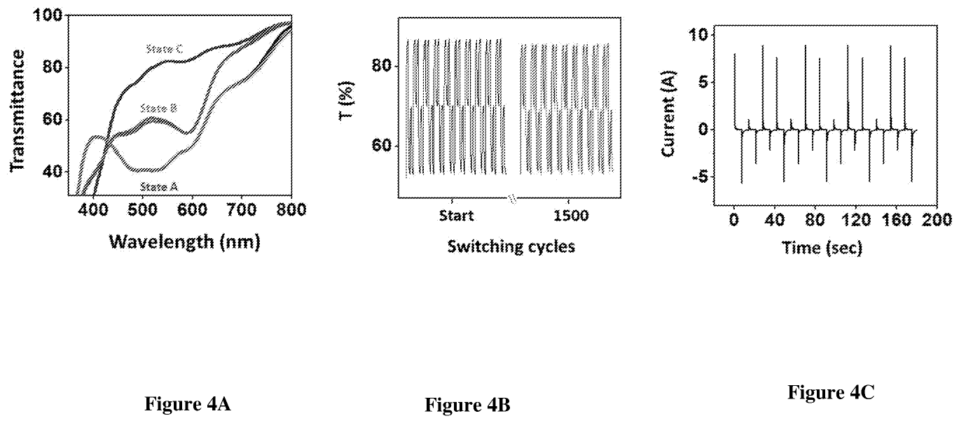

The term ‘transmittance change’ is interchangeable with the ‘contrast ratio’, (ΔT %). ΔT % reflects the change in transmittance when comparing transmittance of two states at a certain wavelength. The change is reported as a percent change (see for example FIG. 4A).

The oxidized and reduced states of the metal-coordinated organic complex or complexes affects the state of the device in terms of absorption spectra. Accordingly, in some embodiments, the state of the device is referred to as ‘the oxidized state of the device’ or as ‘the reduced state of the device’ and this conforms with the state of the metal-coordinated organic complex(es) within the device.

In one embodiment, conductive means electrically-conductive.

In one embodiment, this invention provides a switch comprising the device of the invention as described herein above. Switches of this invention in one embodiment, are referred to as optical switches or as electro-optical (EO) switches, in view of their switchable optical properties.

Absorption spectrum or absorption spectra refer to optical absorption spectrum/spectra as known in the art. Optical absorption refers to the absorption of electromagnetic radiation by the material in one embodiment and as known in the art.

‘Atomization’ in some embodiments, is the first step in a spray-coating process in which bulk liquid converts into small droplets before it is sprayed onto a substrate. In one embodiment, in ultrasonic spray-coating methods, spray nozzles are modified to work with high-frequency ultrasonic waves produced by piezoelectric transducers. These waves create capillary waves. Next, these waves break themselves in tiny droplets when the power supplied by the generator reach the critical height to result in atomization.

In one embodiment, when referring to a case when the metal-coordinated organic complex in said device comprises at least two types of metal ions, it is meant that one molecule of metal-coordinated organic complex comprises one type of ion and another molecule of metal-coordinated organic complex comprises a different type of metal ion. For example, in devices comprising mixtures of complexes selected from e.g. complexes 1 and 2, some molecules comprise Os as the metal ion while others comprise Fe as the metal ion in the complex. The metal-coordinated organic complex thus comprises molecules that are different in the metal ions. Similarly and in one embodiment, the ‘metal-coordinated organic complex’ may comprise different organic molecules, differing by their organic moieties, for example, complexes 2 and 4 can be used as the “metal-coordinated organic complex” in embodiments of this invention. Accordingly and in one embodiment, “metal-coordinated organic complex” can be referred to as “metal-coordinated organic complex(es)” to emphasize that more than one type of complex can be present.

In one embodiment, ‘multi’ means two or more. In one embodiment, ‘multi’ means three or more.

In one embodiment, the term “a” or “one” or “an” refers to at least one. In one embodiment the phrase “two or more” may be of any denomination, which will suit a particular purpose. In one embodiment, “about” or “approximately” may comprise a deviance from the indicated term of +1%, or in some embodiments, −1%, or in some embodiments, ±2.5%, or in some embodiments, ±5%, or in some embodiments, ±7.5%, or in some embodiments, ±10%, or in some embodiments, ±15%, or in some embodiments, ±20%, or in some embodiments, ±25%.

In one embodiment, for a device comprising metal-coordinated organic complex comprising two different metal ions, after applying two oxidative or two reductive voltages, the method further comprising applying a third voltage to said device, thus changing the absorption spectrum of said device back to its initial spectrum or back to its intermediate spectrum. Initial spectrum according to this aspect is the spectrum before any application of voltage. Intermediate spectrum is the spectrum following application of a first voltage and prior to application of a second voltage in one embodiment.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

### EXAMPLES

### Example 1

### Molecular Assembly Formation I

This example describes embodiments of parameters used in formation of devices comprising molecular assemblies.

In this example, ultrasonic spraying system has been used to form metal-organic assemblies MA4 and MA2 on transparent conductive oxides (TCOs) using iron polypyridyl complexes 4 and 2 which enable a better film deposition control and forms more homogeneous films on large TCO surfaces/substrates. Based on the screening experiments, it was concluded that the atomization pressure, distance between the nozzle and substrate, speed of the spray nozzle and number of passes were the most important operational variables influencing the uniformity/morphology of the final film. Parameters of the spray coating process were also dependent on the type of iron polypyridyl complexes 4 or 2 and on the size of the TCOs (results are summarized in Table 1). In general, first, a 1.0 mM solution of PdCl₂(PhCN)₂ in tetrahydrofuran (THF) was sprayed onto FTO/glass or ITO/PET substrates at atomization pressure of 1.03 kPa or 1.30 kPa. Nozzle-to-substrate distance was 5.5 cm and nozzle is moved in a pre-programmed zig-zag pattern along X and Y direction with speed of 5 mm/s with flow rate 0.6 mL/min at room temperature (approximate 23° C.). this step resulted in the formation of a dense layer of palladium. Next, this step was followed by spraying (7 passes) the CH₂Cl₂/MeOH (1:1 v/v) solution of complex 1 (0.2 mM). This deposition sequence was repeated 3× to generate MA4. For complex 2 and mixture of complexes MA4·1, see parameter details in Table 1 herein below.

The spraying system used was an ultrasonic spraying system (Sono-Tek) equipped with two ultrasonic nozzles (having 2 mm-6 mm diameter spray areas, operating at 120 kHz), which were mounted onto an X-Y-Z movable scanner.

Each compound (linker or organic complex) was sprayed between 1−10 passes. “Pass” means a spray event. For example, 3 spray passes refer to a substrate that was sprayed 3 consecutive times with a certain compound.

Each complete spray-deposition of a linker and a complex provides one deposition cycle. Repetition means how many deposition cycles have been performed. For example, 3 repetitions mean 3 layers of (linker+complex).

The number of spraying passes means how many times a nozzle was used to spray a certain solution on to the substrate. For example, 7 (PdCl₂) and 7 (4) passes means that first PdCl₂ was sprayed for a total of 7 times, followed by 7 times spraying of complex (4); This is equal to one deposition cycle.

‘Repetition’ means how many times the above deposition cycle was repeated to obtain the film. For example, 3 deposition cycles (each cycle of linker+complex) mean 3 repetitions.

In embodiments of this invention, processing time for the formation of a complete 2 cm×2 cm film is about 45 min.

TABLE 1

| Entry | Spray parameters | MA4^{b, c} | MA4^{b, c} | MA2^{b, d} | MA2^{b, d} | MA4 · 1^{e} |
|---|---|---|---|---|---|---|
| 1 | Substrate dimensions | 2 cm × 2 cm | 6 cm × 6 cm | 2 cm × 2 cm | 6 cm × 6 cm | 2 cm × 2 cm |
| 2 | Nozzle to substrate | 5.5 cm | 5.5 cm | 5.5 cm | 5.5 cm | 5.5 cm |
| | distance (cm)^{a} | | | | | |
| 3 | Atomization | 1.03 | 1.30 | 1.30 | 1.30 | 1.30 |
| | pressure (kPa)^{a} | | | | | |
| 4 | Flow rate (mL/min)^{a} | 0.6 | 0.6 | 0.6 | 0.6 | 0.6 |
| 5 | Nozzle speed (mm/s)^{a} | 5 | 5 | 5 | 5 | 5 |
| 6 | Number of passes | 7 (PdCl₂) | 3 (PdCl₂) | 10 (PdCl₂) | 6 (PdCl₂) | 8 (PdCl₂) |
| | | 7 (4) | 3 (4) | 5 (2) | 3 (2) | 5 (4 · 1) |
| 7 | Repetition^{a} | 3 | 4 | 3 | 3 or 4 | 3 |

^{a}the same conditions were used for both the nozzles.
^{b}THF solution of PdCl₂(PhCN)₂ (1.0 mM).
^{c}CH₂Cl₂/MeOH (1:1 v/v) solution of complex 4 (0.2 mM).
^{d}CH₂Cl₂/MeOH (1:1 v/v) solution of complex 2 (0.3 mM).
^{e}equimolar solution of complexes 4 and 1 in CH₂Cl₂/MeOH (1:1 v/v) at a final concentration of 0.2 mM was used for the formation of MA4 · 1.

### Example 2

### Molecular Assembly Formation II

This example describes one embodiment of formation of devices comprising molecular assemblies (MA), the assemblies comprise metal ion coordinated complex 4 and metal ion coordinated complex 1 and are herein referred to as MA4·1. The two metal ion species are iron (Fe, 4) and osmium (Os, 1). The MA4·1 showed multi-color electrochromic behavior enabled by the different redox potential of the two metal ions used. The molecular assemblies (MA4·1) were formed on glass substrates coated by fluorine-doped tin oxide (FTO). The substrate size was 2 cm×2 cm. The MA4·1 were applied to the coated substrates by spray coating a solution of 1:1 mixture of two complexes Fe (4, 0.2 mM) and Os (1, 0.2 mM) in DCM/MeOH.

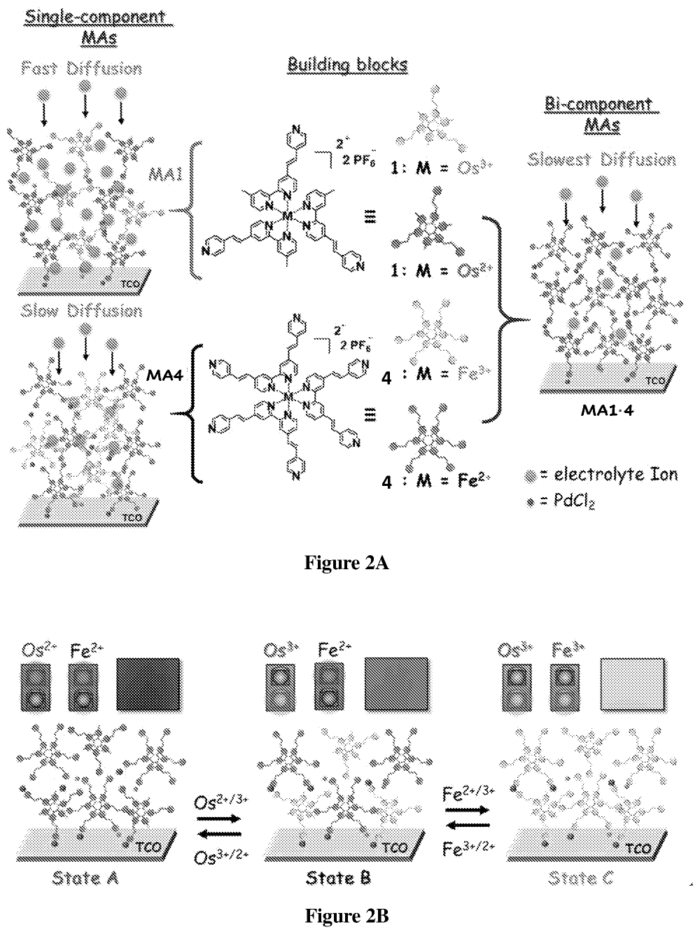

Prior to MA4·1 application, a 1.0 mM solution of PdCl₂(PhCN)₂ in tetrahydrofuran (THF) was spray-coated onto FTO/glass. This was followed by spray coating a 0.2 mM solution of the 1:1 mixture of two metal-complexes (Fe and Os). 3 repetitions of this procedure have been performed. Finally, the MA4·1 spray-modified TCOs were rinsed with acetone to remove the unbound material from the surface and were dried under a gentle stream of air. The combination of these two complexes (4 and 1) gave red color molecular assemblies (MA4·1) on FTO/glass (2 cm×2 cm). Photograph is shown in FIG. 2B. The intensity of the color is a function of the number of deposition cycles.

In this example, 3 repetitions of deposition cycles were used (see Table 1, entry 7 corresponding to MA4·1).

In this example, films were rinsed with acetone after all deposition cycles have been completed (not after each spraying step). For each layer (linker or complex) few spray passes have been performed, e.g. 8 (PdCl₂) and 5 (4·1) passes, see table 1 herein above.

Example 3

### Molecular Assembly Characterization

This example describes the multi-color electrochromic behavior of devices comprising molecular assemblies (MA). The UV/vis spectroscopy of MA4·1 showed two metal-to-ligand charge transfer (MLCT) bands at λₘₐₓ₁=530 nm (Os complex) and λₘₐₓ₂=592 nm (Fe complex). The scanning electron microscope (SEM) measurements showed uniform and porous surface morphology of [MA4·1|FTO/glass]. The thickness of the MA4·1 was found to be ˜184±62 nm with root-mean-square roughness (rms) of 55 nm for the measured scan area of 5 μm×5 μm. Film thickness was evaluated from an SEM image showing the cross section of the molecular assemblies, milled with a 30 keV Ga⁺, focused ion beam (FIB). The root-mean-square roughness values were obtained by atomic force microscopy (AFM).

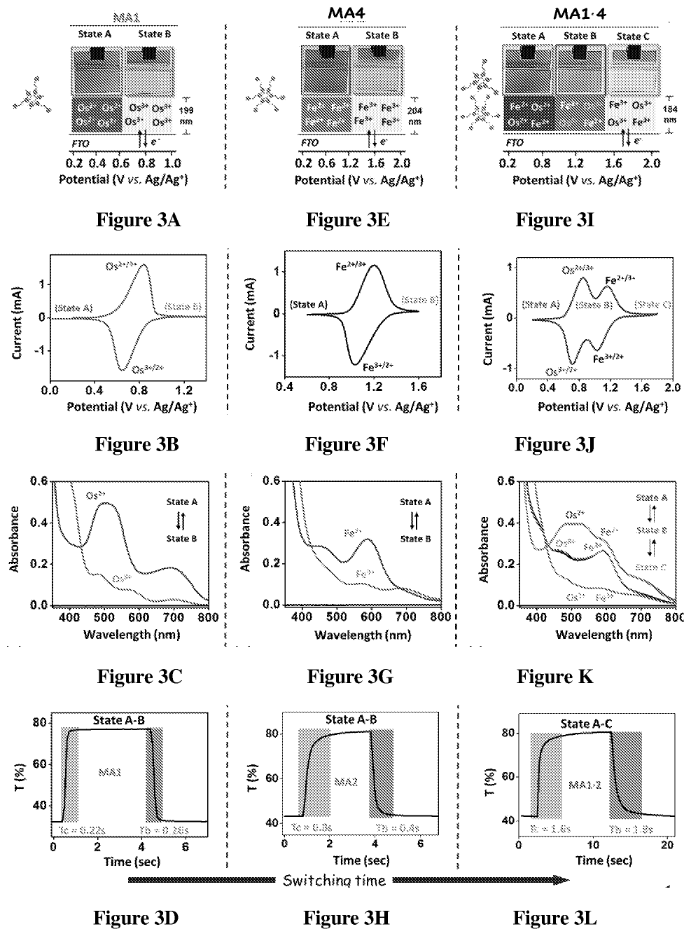

The multi-color electrochromic and electrochemical properties of the [MA4·1|FTO/glass] films were analyzed in 0.1 M TBAPF₆/acetonitrile electrolyte. The [MA4·1|FTO/glass] film exhibit two well-defined redox states with a wide potential separation, as a result, three distinct redox states: “A”, “B”, and “C” were observed during electrochemical study (FIG. 3J-3L). In state “A” (red color, FIG. 3I left), both metal ions Fe²⁺ and Os²⁺ are present in +2 oxidation state. In state “B” (gray color, FIG. 3I middle), at 0.8 V applied potential, Os²⁺ metal ion was oxidized to Os³⁺. Since iron metal ion still present as Fe²⁺ at this potential, this results in gray color to the film. When both, Fe²⁺ and Os²⁺ metal centers were oxidized to Fe³⁺ and Os³⁺ in state “C” at 1.8 V applied potential, this contributed to a colorless film (FIG. 3I, right). Similar color changes were also observed in absorption spectral changes of [MA4·1|FTO/glass] upon application of various potentials from 0.2 to 1.8 V. Gradual increase in the potential from 0.2 to 0.85 V, prompt oxidation of the Os²⁺ metal ion. Oxidation of osmium metal selectively results in the decrease of MLCT band corresponding to Os²⁺ center at λₘₐₓ₁=530 nm and absorption spectra corresponding to Fe²⁺ complex was observed with MLCT band at λₘₐₓ₂=592 nm (middle trace FIG. 3K) and the red color of the film changed to gray. When the potential further increased to 1.8 V (Ox-2_1.80V), oxidation of Fe²⁺ to Fe³⁺ results in decrease in MLCT band at λₘₐₓ₂=592 nm and color of film changed from gray to colorless (lower trace FIG. 3K). It should be noted that three states (red to gray to colorless) were fully reversible when the applied potentials were reversed, these color changes are clearly visible to the eye (FIGS. 3A, 3E, 3I).

Transmittance spectral changes also supported that three states (red to gray to colorless) are fully reversible and displays the highest contrast ratios (ΔT, 34%) at λ=530 nm (FIG. 4A). Spectroelectrochemical (SEC) stability of the film was measured by switching between: (i) 0.2-1.8 V, (ii) 0.2-0.8 V, and (iii) 0.75-1.8 V. The transmittance change (or contrast ratio, ΔT %) was monitored at λₘₐₓ=530 nm. The highest ΔT values is ˜34% while switching between state “A” to “C” at 0.2-1.8 V, while switching between state “A” to “B” and “B” to “C” at the applied potential: (i) 0.2-0.8 V and (ii) 0.75-1.8 V, the ΔT value is ˜17%. The switching stability of the film was about 2000 cycles and the contrast ratio (ΔT %) is as high as in the initial cycles (FIG. 4B-4C).

### Example 4

### Laminated Devices

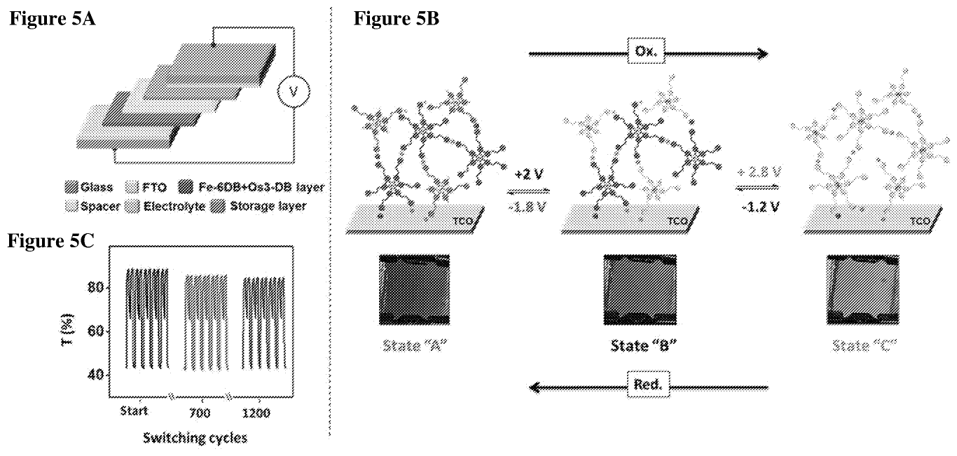

Laminated devices were fabricated using a film of MA4·1 on FTO/glass as working electrode (WE) (see details in examples 1 and 2 herein above). The counter electrode (CE) was prepared by spin-coating a thin layer of poly(3,4-ethylenedioxythiophene)-poly(styrenesulfonate) (PEDOT:PSS) on FTO/glass. The electrolyte was 90:7:3 wt % acetonitrile (ACN)/poly(methyl methacrylate) (PMMA)/lithium perchlorate salt (LiClO₄) based gel electrolyte. The electrolyte provides ionic conductive medium to the device. Herein, the thin layer of PEDOT:PSS acts as charge storage layer for the laminated device (FIG. 5A). A spacer was used in this example (FIG. 5A). The spacer was a frame of 210-μm-thick double-sided tape (3M™ 9088), an insulating frame used to prevent short circuit between the working and the counter electrodes and provided for holding the electrolyte in the laminated set-up.

Spectroelectrochemical (SEC) switching of the laminated device was measured by switching between (i) −1.8 V to +2.8 V and by switching between (ii) −1.2 V to +2.8 V. The transmittance change of the device was measured using UV/vis spectrophotometer at λmax=530 nm.

Similar to above, in state “A” (red color), both metal ions are present in Fe²⁺ and Os²⁺ oxidation state. Upon the application of +2 V, Os²⁺ metal centers were selectively oxidized to Os³⁺ (keeping Fe²⁺), contributing to gray color state “B”. When the applied potential increased to +2.8 V both Fe²⁺ and Os²⁺ and metal center were oxidized to Fe³⁺ and Os³⁺, resulted in fully bleached or colorless state “C”. It should be noted that the three states (red to gray to colorless) are fully reversible and clearly visible to the eyes. Therefore, when the applied potential is reversed to −1.2 V, Fe³⁺ metal ion reduced back to Fe²⁺ and gray color state “B” re-appeared and at −1.8 V, Os³⁺ also reduced to Os²⁺ which contributed to red color state “A” (FIG. 5B). SEC stability of the laminated device was measured by switching the device for up to 1500 redox cycles using double-potential-step chronoamperometry: (i) −1.8 V to +2.8 V, and (ii) −1.2 V to +2.8 V, at λₘₐₓ=530 nm. The highest contrast ratio (ΔT) value is ˜46% while switching between state “A” to “C” (from −1.8 V to +2.8 V). Switching between state “B” to “C” (from −1.2 V to +2.8 V), the ΔT value is ˜23%. The switching stability of the device is for at least 1200 redox cycles while maintaining 90% of initial contrast ratio (FIG. 5C).

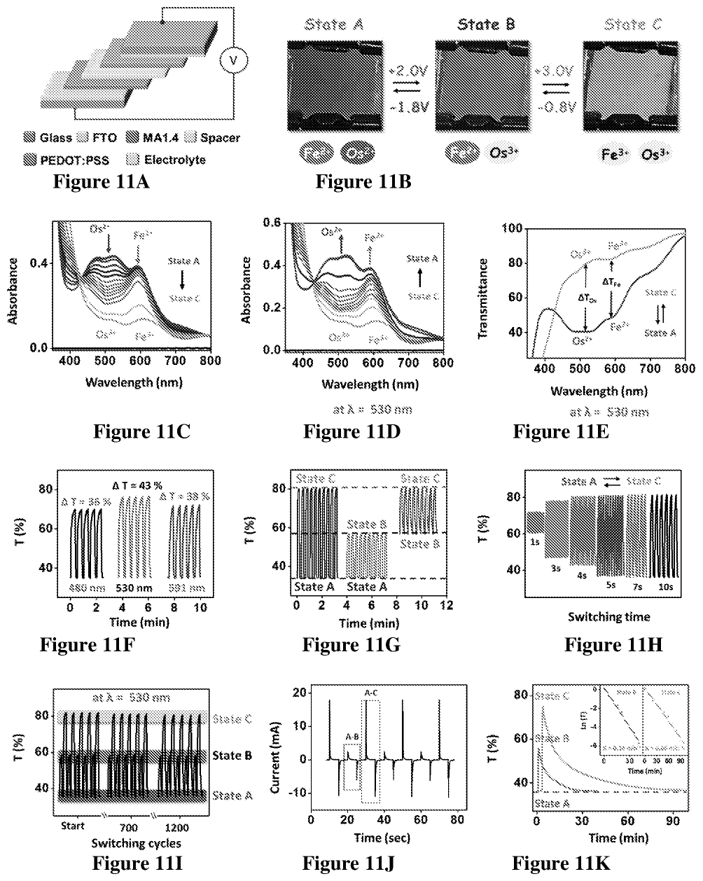

Note that the voltage connections in FIGS. 5A and 11A are drawn as connecting to the glass. However, the connections actually contact the conductive portion of the substrates. This was omitted for clarity in view of the small dimension illustrated for the FTO layer.

### Example 5

### Assemblies MA1·4, MA2·4 and MA2·3

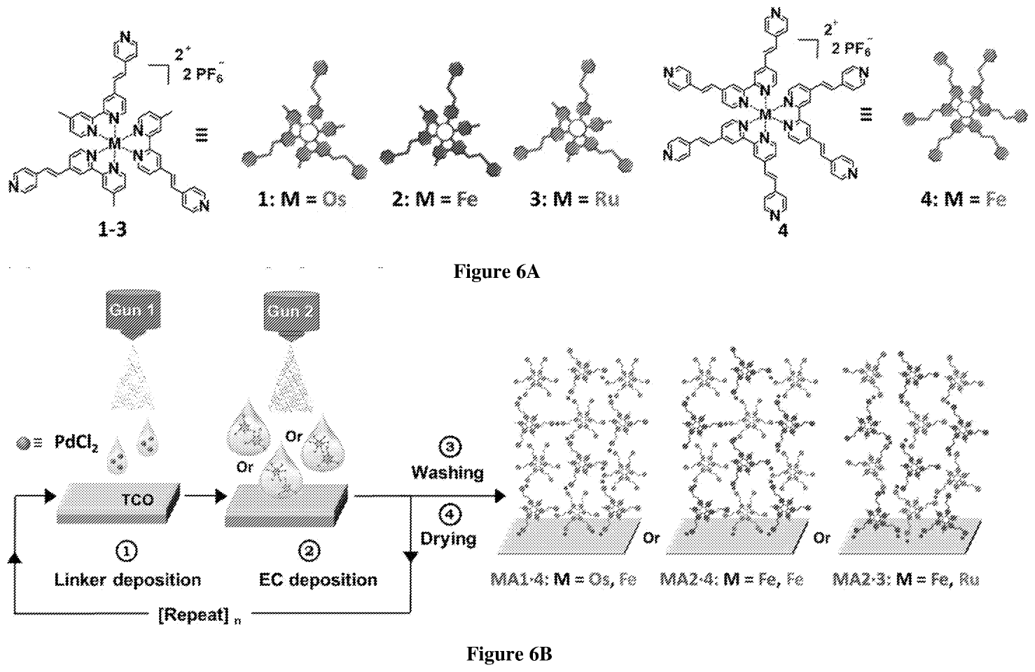

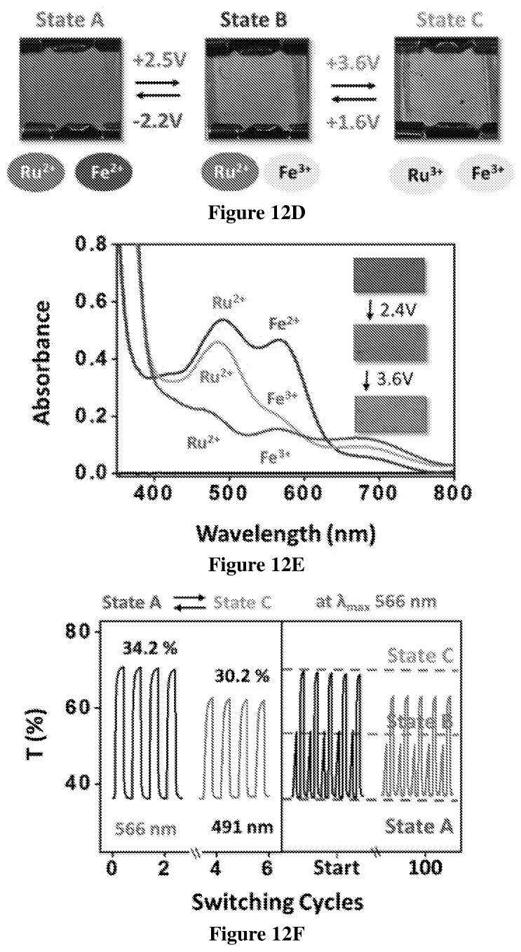

Coordination network of polypyridyl complexes have shown excellent two-state electrochromic performance by switching between colored and bleached states due to reversible one electron redox reaction. The switching reflects a change in the intensity of the metal-to-ligand charge transfer (MLCT) bands. In an effort to expand the scope of these assemblies to multi-color ECD's, mixtures of various polypyridyl complexes were used to form assemblies on transparent conducting oxides (TCOs) surfaces thus forming multi-responsive electrochromic assemblies MA1·4, MA2·4 and MA2·3 (FIGS. 6A-6B). The fluorine-doped tin oxide (FTO) glass surfaces were fabricated by spray-coating solutions of PdCl₂(PhCN)₂ (1 mM or 2 mM, THF) and equimolar mixture of F1·4, 2·4, and 2·3 (0.2 mM each, DCM/MeOH, 1:1 v/v) using a commercially available automated set-up to afford 3-D network of metallo-organic assemblies (MA); (for more details see example 7 herein below). A palladium salt was used to generate a polymeric network on the surface by coordination of the vacant pyridine group of the metal complexes (FIGS. 6A-6B). All molecular assemblies (MA1·4, MA2·4 and MA2·3) were characterized by UV-vis spectroscopy, X-ray photoelectron spectroscopy (XPS), scanning electron microscopy (SEM) and electrochemistry (FIGS. 13A-13L; 14A-14H; 15A-15E). The micro-scale SEM and AFM measurements of MA's confirmed that the surface is grainy and homogenously covered. The root-mean-square roughness for 5 μm×5 μm scan areas calculated from AFM imaging is 38-55 nm for MAs (FIG. 13A, 14A and 15A). The thickness and inner structure of the MAs were determined by milling with 30 keV Ga+ focused ion beam (FIB). The thickness of MA was found to be ˜184±62 nm (MA1·4), to ˜187±67 nm (MA2·4) and 184±30 nm (MA1·4) (FIG. 13C, 14D and 15C). XPS analysis of MAs revealed that the Pd content is very close to the expected ratio for a fully formed network, apparently, the MA are expected to be fully cross-linked (FIG. 13D).

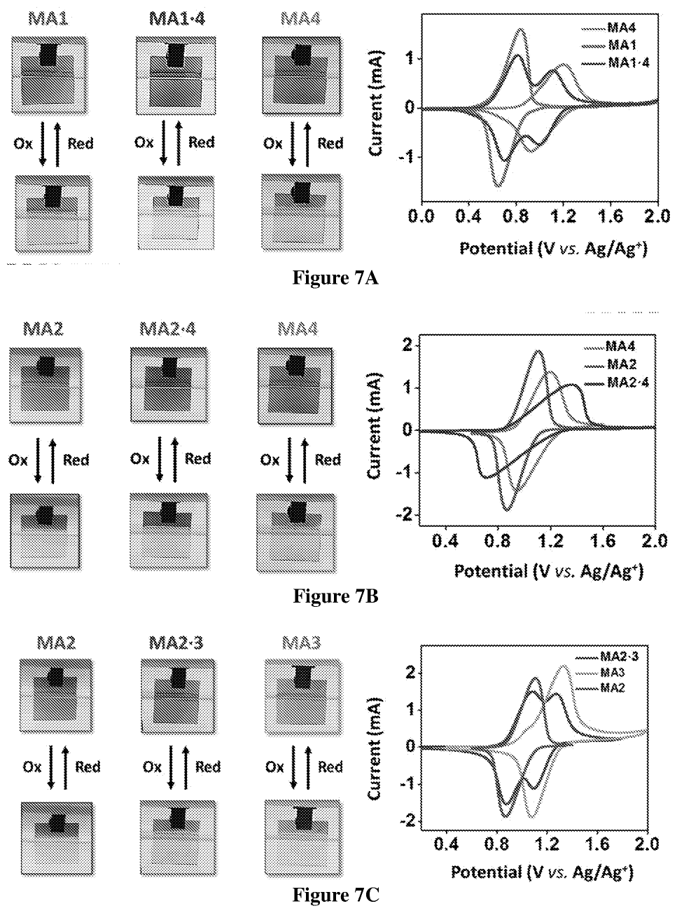

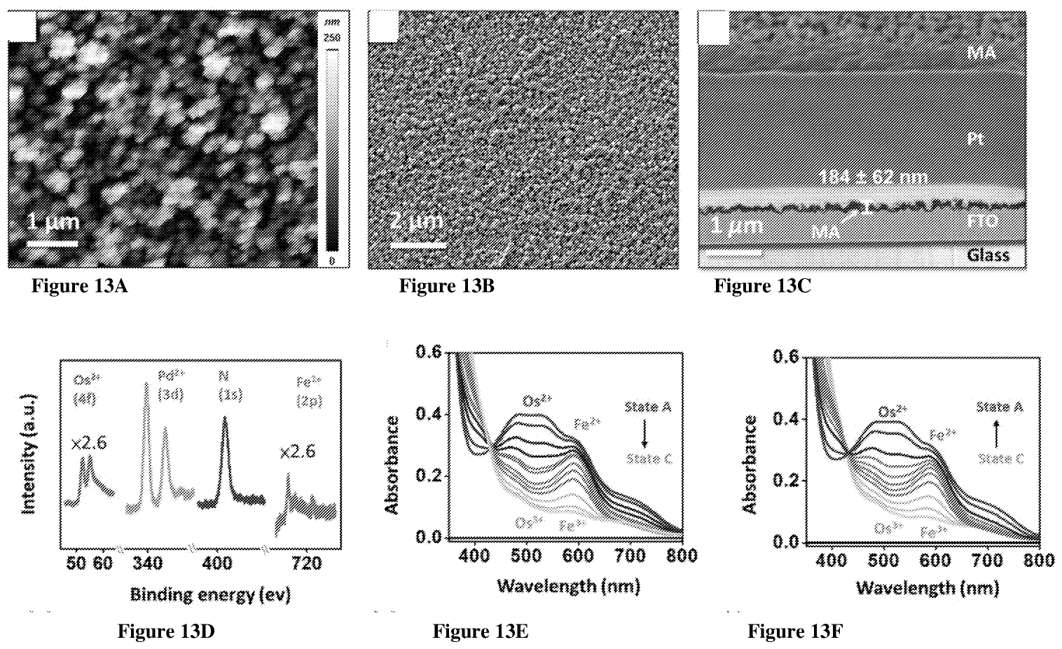

In this study we report three molecular assemblies MA1·4, MA2·4 and MA2·3 formed from equimolar solution of 1·4, 2·4 and 2·3 complexes, have the following color: red (MA1·4), blue (MA2·4), red (MA2·3) and shows metal-to-ligand charge transfer (MLCT) bands at λ=491-585 nm. An intense π-π* transition band is also present at λ=˜332 nm (FIGS. 7A-7C, 8A-8C, 13E, 14D and 15D). First, multi-stimuli response of the MAs films were investigated by cyclic voltammetry in 0.1 M solution of TBAPF₆ in acetonitrile (ACN) as the supporting electrolyte, with FTO, Pt wire, and Ag wire as the working, counter and reference electrodes, respectively at a scan rate of 100 mV/s. The CV of [MA1·4|FTO/glass] films exhibit two well-resolved and reversible redox processes having half-wave potentials at 0.77 and 1.08 V, corresponding to Os^{2+/3+} (first redox couple) and Fe^{2+/3+} (second redox couple), respectively. Similarly, CV of [MA2·3|FTO/glass] shows a similar trend having two half-wave potentials at 0.97 and 1.18 V, corresponding to Fe^{2+/3+} (first redox couple) and Ru^{2+/3+} (second redox couple), respectively. The CV of [MA2·4|FTO/glass] exhibit only one well-resolved redox process having half-wave potentials at 1.03 V (FIG. 7A-7C).

Spectroelectrochemistry

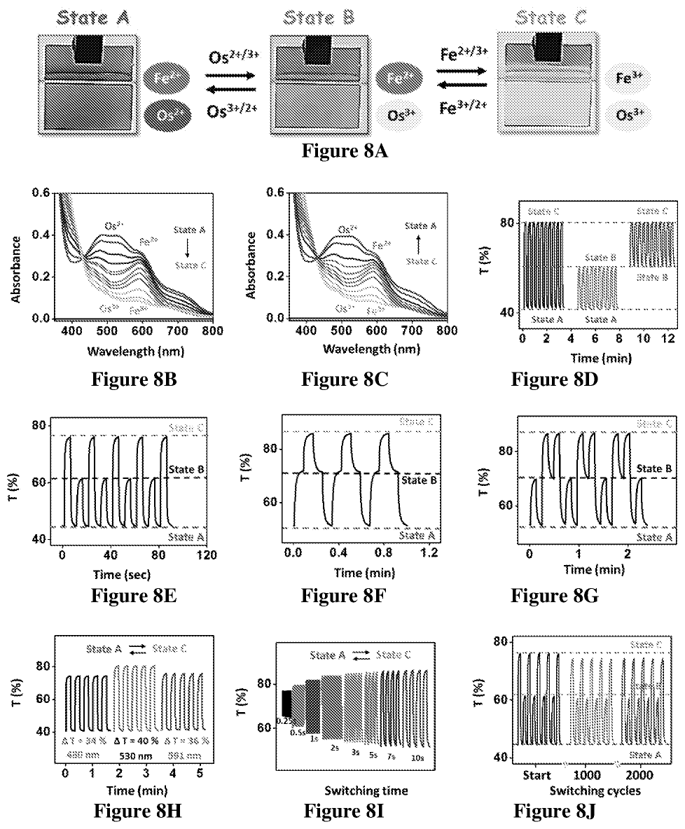

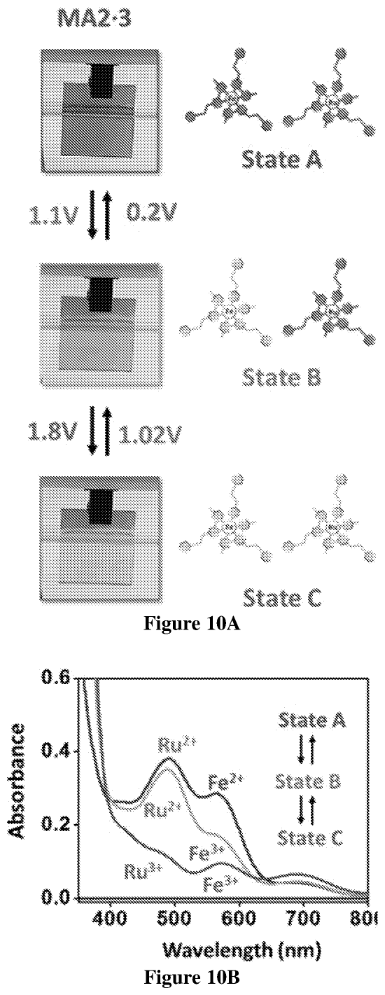

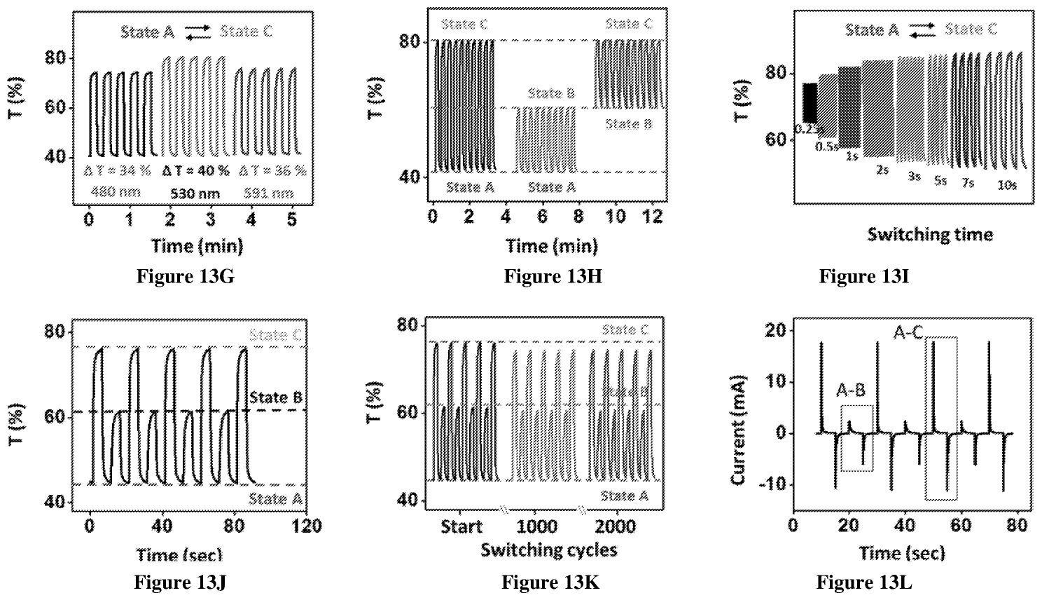

The spectral response of MA1·4, MA2·4 and MA2·3 films on FTO/glass substrate were monitored upon applying the potentials from 0.2-1.8 V (FIGS. 8A-8C). For [MA1·4|FTO/glass], in state A (red color), both metals ion Fe²⁺ and Os²⁺ are present in +2 oxidation state. Upon steadily increasing the potential from 0.2 V to 0.8 V, color of the film changes to gray (state B). In state B: Os²⁺ metal ion oxidized to Os³⁺, at 0.8 V iron metal ion still present as Fe²⁺ resulted gray color to the film. Further increase in the potential to 1.8 V resulted in oxidation of Fe²⁺ to Fe³⁺ and film become colorless (state C) due to oxidation of both metal center (Os^{2+/3+} and Fe^{2+/3+}). It should be noted that three states (red-gray-colorless) were fully reversible when the applied potentials were reversed, these color changes are clearly visible to the eyes (FIG. 8A). The UV/vis absorption spectroscopy of [MA1·4|FTO/glass], shows metal-to-ligand charge transfer (MLCT) bands at λ=480, 530 and 705 nm, with Os and λ=592 nm, with Fe (FIG. 8A). Gradual increase in the potential from 0.2 to 0.8 V, decreased the MLCT band corresponding to Os²⁺ center at λ=480, 530 and 705 nm, and MLCT band corresponding to Fe²⁺ complex at λ=592 nm, appear predominantly and color of the film become gray. Further increase in the potential to 1.8 V resulted in bleaching of MLCT band at λ=592 nm corresponding to Fe²⁺ center, and color of the film changed from gray to colorless (FIG. 8A). Similarly, the color of the [MA2·4|FTO/glass] film changing from blue to gray to colorless, and for [MA2·4|FTO/glass] film, red to orange to colorless upon sweeping the potentials from 0.2-1.8 V (FIG. 10). Further, these changes are supported by UV/vis absorption spectral changes of MA2·4 and MA2·3, which show decrease in MLCT bands. The MAs have highest contrast ratios (ΔT, %) at the λₘₐₓ values corresponding to their MLCTs: 40% (MA1·4), 43% (MA2·4) and 34% MA2·3 (FIG. 13G, 14F and 15E). Delightfully, these multi-color [MAs|FTO/glass] films can be switched reversibly in different states: A-C, A-B, and B-C, without degrading the MAs (FIGS. 13H and 15E) with spectroelectrochemical (SEC) stability up to 2000 cycles (MA1·4), 1200 cycles (MA2·4) and 50 cycles (MA2·3), using double-potential-step chronoamperometry, respectively (FIG. 13K, 14G and 15E).

### Example 6

### Electrochromic Properties of Laminated Devices

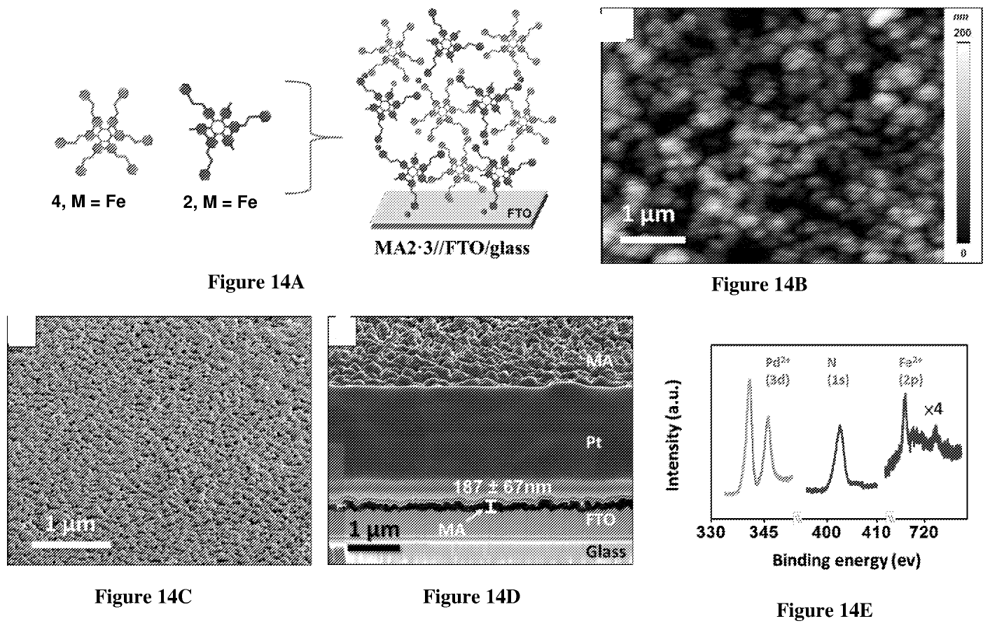

The laminated electrochromic devices were constructed by using [MAs|FTO/glass] as working electrode and a thin layer of PEDOT:PSS covered FTO as counter electrode, where PEDOT:PSS layer act as charge storage layer. The working and counter electrodes are separated by a LiClO₄/PMMA based gel-electrolyte and a double-sided tape as spacer (FIG. 11A). Photographs of multi-ECD based on [MA1·4|FTO/glass] film with three redox states (red-gray-colorless) shown in FIG. 11B, red color of the device changes to gray and to colorless under the application of various potentials (−1.8 V to +3.0 V) and colors reverse back upon reserving the potential (+3.0 V to −1.8 V). UV/vis measurements clearly show the corresponding reversible changes in the spectral intensities of the MLCT bands corresponding to Os (530 nm and 703 nm) and Fe (592 nm) complex of the laminated device. The gradual increase in the potential of laminated device, indicating the complete oxidation of Os^{2+/3+} at +2.0 V and MLCT band at 530 and 703 nm disappeared, and oxidation of Fe^{2+/3+} at +3.0 V resulted in the decrease of MLCT band corresponding to 592 nm (FIG. 11C). The MA1·4, ECD shown excellent reversibility after sweeping of the potential from +3 V to −1.8 V, MLCT band corresponding to iron complex (592 nm) reappeared at −0.8V and device is fully reduced back at −1.8 V (FIG. 11D). Transmittance spectra of the fully reduced state (red colored) and fully oxidized state (bleached) displays highest contrast ratios (ΔT 43%) at λ=530 nm (FIG. 11E). Further, the transmittances of MA1·4 multi-ECD monitored between state A-C (potential was swiped between −1.8 V to +3V) as a function of different wavelength with 20 s pulse width, and at λ=530 nm; maximum contrast ratio (=43%) was obtained with response time ˜4.4 s, which is much faster than many of the reported laminated devices (FIG. 11 F,G). These devices have good reversible switching between different states: A-C (−1.8 V to +3V), A-B (−1.8 V to +2 V), and B-C (−0.8 V to +3 V) (FIG. 11H). The spectroelectrochemistry (SEC) stability performed by alternatively two-state switching between, A-B (ΔT %, ˜45%) and A-C (ΔT %, ˜23%), using double-potential-step chronoamperometry: (i) −1.8 V to +3V, and (ii) −1.8 V to +2V, with 20 s pulse width. The initial ΔT value remains stable for at least 1200 redox cycles (FIG. 11I). Similar, stability was observed when device alternatively switched between state (i) A-C, and (ii) B-C for more than 1200 cycles (FIG. 16D). The ability to maintain the transmittance value after the applied potential is switched off was also tested for the laminated devices to verify its applicability in information storage. The measured decay time for state B and C, when the applied potential +2 V and +3 V turned off, were found ˜25 min and ˜90 min, respectively (FIG. 11J). The observed decay kinetics for state B and C are 0.14 min⁻¹ and 0.06 min⁻¹, respectively (FIG. 11K). These values are higher than reported for many electrochromic metal oxides and some of the best-performing organic polymers.

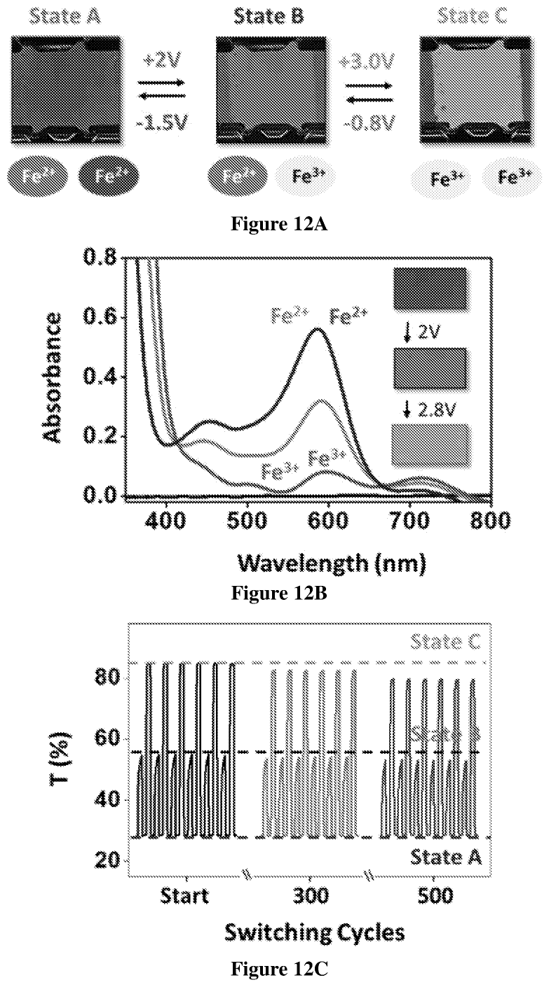

Similarly, electrochromic properties of MA2·4 and MA2·3-coated FTOs were investigated in laminated set-up using PEDOT:PSS as counter-electrode in LiClO₄/PMMA/ACN gel electrolyte. Photographs of these two devices and representative optical and spectroelectrochemical data are shown in FIGS. 12A-12F. The spectral response was monitored upon applying the potentials from (−1.8 V) to (+2.8 V) for MA2·4 and from (−2 V) to (+3.6 V) for MA2·3. Gradual increase in the potential prompt decrease of the MLCT band at λₘₐₓ=585 nm for MA2·4 and device color change from blue (−1.8 V) to gray (+2 V) to colorless (+2.8 V). Similarly, for MA2·3 device, MLCT band at λ=566 nm (with Fe), disappeared at +2.4 V and color of the device changed from red to orange. Further increase in potential up to +3.6 V resulted the decrease of the MLCT band at λ=491 nm (with Ru), and the device turns colorless. Investigation of the electrochromic properties of MA2·4 and MA2·3 in laminated set-up revealed a contrast ratios: of 57% for MA2·4 (at λₘₐₓ=585 nm) and for MA2·3; 34% (at λₘₐₓ=566 nm) and 30% (at λₘₐₓ=491 nm) respectively (FIG. 12A-12F).

### Example 7

### Materials and Methods

Solvents (AR-grade) were purchased from Bio-Lab (Jerusalem), Frutarom (Haifa, Israel), or Mallinckrodt Baker (Phillipsburg, N.J.). Poly(methyl methacrylate) (PMMA), lithium perchlorate, and PdCl₂(PhCN)₂ were purchased from Sigma-Aldrich. Fluorine-doped tin oxide-(FTO)-coated glass substrates (6 cm×6 cm, Rs=8-12Ω/□) and indium-tin oxide (ITO)-coated poly(ethylene terephthalate) (PET) substrates (10 cm×10 cm, Rs=10, 30, 60Ω/□) were purchased from Xinyan Technology Ltd. (Hong Kong, China). FTO-coated glass substrates were cleaned by sonication in ethanol for 10 min, dried under a stream of N₂, and subsequently cleaned for 20 min with UV and ozone in a UVOCS cleaning system (Montgomery, Pa.). The substrates were then rinsed with tetrahydrofuran (THF), dried under a stream of N₂, and oven-dried at 130° C. for 2 h. ITO-coated PET substrates were cleaned by immersing for 30 s in ethanol and acetone and then drying under a stream of air.

UV/Vis Spectroscopy. UV/vis spectra were recorded on a Cary 100 spectrophotometer. The absorbance was measured using the Cary Win UV-Scan application program, version 3.00 (182) by Varian (200-800 nm), whereas the transmittance was measured using the Cary Win UV-Kinetics application program, version 3.00 (182) by Varian. Bare substrates were used to compensate for the background absorption.

X-ray Photoelectron Spectroscopy. XPS measurements were carried out on FTO/glass substrates (2.0 cm×2.0 cm) with a Kratos AXIS ULTRA system, using a monochromatic Al Kα X-ray source (hν=1486.6 eV) at 75 W and detection pass energies ranging between 20 and 80 eV. Curve-fitting analysis was based on Shirley or linear background subtraction and application of Gaussi an-Lorenzi an line shapes.

Atomic Force Microscopy (AFM). AFM imaging was carried out by using a JPK AFM (JPK Nanowizard III, Berlin, Germany). Scans of 512×512 pixels per image were made in QI mode using a Quartz like probe (qp-BioAC CB1) with a spring constant of 0.15-0.55 N/m.

Focused Ion Beam (FIB) Microscopy. SEM images were recorded using a Helios 600 FIB/SEM dual-beam microscope (FEI), operating at 5 keV. The images were taken at the surface of the samples and at cross sections that were milled with a 30 keV Ga⁺ focused ion beam (FIB). MA|FTO/Glass 10Ω/□ was first coated with a 3-nm-thick layer of iridium, followed by coating a 150-200 nm-thick layer of platinum using electron-beam-assisted deposition. This process was followed by anion-beam-assisted deposition of a 500-600-nm-thick layer of platinum. The platinum coating protects the MA from ion-beam damage.

Electrochemical Characterization of multi-color electrochromic films. Electrochemical experiments were carried out using a CHI660A or a CHI760E electrochemical workstation. The electrochemical cell consisted of the MA on FTO/glass substrates (1 cm×2 cm or 2 cm×2 cm) serving as the working electrode, Ag/Ag⁺ was used as the quasi-reference electrode, and a Pt wire was used as the counter electrode. Tetrabutylammonium hexafluorophosphate (TBAPF₆) in ACN (0.1 M) was used as the supporting electrolyte.

Formation of molecular assemblies (MAs). Spray coating was performed with an automatic Ultrasonic Spraying System (Sono-tek) equipped with two ultrasonic nozzles (having 2 mm-6 mm diameter spray areas, operating at 120 kHz), which were mounted onto an X-Y-Z movable scanner.

Formation of bimolecular assemblies (MA1·4 and MA2·4) using a mixture of complexes 1·4 and 2·4. The MA1·4 and MA2·4 were obtained by automated ultrasonic spray-coating of solutions of PdCl₂(PhCN)₂ and equimolar mixtures of complexes 1·4 and 2·4, respectively, at an atomization pressure of 1.30 kPa. The nozzle-to-substrate distance was 5.5 cm and the nozzle was moved in a pre-programmed pattern along the X and Y direction at a speed of 5 mm/s and with a flow rate of 0.6 mL/min at room temperature (˜23° C.). A THF solution of PdCl₂(PhCN)₂ (1.0 mM) and an equimolar CH₂Cl₂/MeOH (1:1 v/v) solution of complexes 1·4 and 2·4 (0.2 mM each) were used for the formation of the MA1·4 and MA2·4. The solution of PdCl₂(PhCN)₂ (1.0 mM) was sprayed onto the substrate (8 passes), which was followed by spraying (5 passes) the mixture of metal complexes solution (0.2 mM). This deposition sequence was repeated 3× to generate the MA1·4 and MA2·4. The substrates were then immersed in acetone for 30 s and were dried under a gentle stream of air (Table 2).

Formation of bimolecular assemblies (MA2·3) using a mixture of complexes 1·4. The MA2·3 was obtained by automated ultrasonic spray-coating of solutions of PdCl₂(PhCN)₂ and equimolar mixtures of complexes 2 and 3, at an atomization pressure of 1.30 kPa. The nozzle-to-substrate distance was 5.5 cm and the nozzle was moved in a pre-programmed pattern along the X and Y direction at a speed of 5 mm/s and with a flow rate of 0.6 mL/min at room temperature (˜23° C.). A THF solution of PdCl₂(PhCN)₂ (2.0 mM) and an equimolar CH₂Cl₂/MeOH (1:1 v/v) solution of complexes 2 and 3 (0.2 mM each) were used for the formation of the MA2·3. The solution of PdCl₂(PhCN)₂ (2.0 mM) was sprayed onto the substrate (10 passes), which was followed by spraying (5 passes) the mixture of metal complexes solution (0.2 mM). This deposition sequence was repeated 3× to generate the MA2·3. The substrates were then immersed in acetone for 30 s and were dried under a gentle stream of air (Table 2).

Fabrication of laminated multi-color electrochromic devices. A layered architecture was used to construct laminated sandwich cells based on FTO/glass substrates (2 cm×2 cm) coated with MAs serving as working electrodes. PEDOT:PSS-coated FTO/glass substrates (2 cm×2 cm) were used as reference and counter electrodes, respectively. A solution of PEDOT:PSS and isopropyl alcohol (1:1.4 v/v) was drop-casted onto FTO/glass substrates. Subsequently, the substrate was spun at 500 rpm for 10 s and then at 1000 rpm for 30 s. Next, the substrate was heated in an oven at 120° C. for 1 min. A frame of 210-μm-thick double-sided tape (3M 9088) was attached to the working electrode, leaving an exposed edge (1-2 mm) for copper tape contacts. Contacts were also connected to an edge (1-2 mm) of the counter electrode. The two electrodes were placed with the two conducting faces facing each other. The electrolyte gel (90:7:3 wt % ACN/PMMA/lithium perchlorate salt) was injected using a syringe between the two electrodes.

TABLE 2
Spray coating parameters for fabricating electrochromic assemblies
(MA1, MA3, MA1 · 4, MA2 · 4 and MA2 · 3) on FTO/glass (2 cm × 2 cm).

| Entry | Spray parameters | MA1^{b, d} | MA3^{c, d} | MA1 · 4^{b, d} | MA2 · 4^{b, d} | MA2 · 3^{c, d} |
|---|---|---|---|---|---|---|
| 1 | Nozzle to substrate | 5.5 cm | 5.5 cm | 5.5 cm | 5.5 cm | 5.5 cm |
| | distance (cm)^{a} | | | | | |
| 2 | Atomization (kPa)^{a} | 1.30 | 1.30 | 1.30 | 1.30 | 1.30 |
| 3 | Flow rate (mL/min)^{a} | 0.6 | 0.6 | 0.6 | 0.6 | 0.6 |
| 4 | Nozzle speed (mm/s)^{a} | 5 | 5 | 5 | 5 | 5 |
| 5 | Number of passes | 10 (PdCl₂) | 10 (PdCl₂) | 8 (PdCl₂) | 8 (PdCl₂) | 10 (PdCl₂) |
| | | 5 (1) | 5 (3) | 5 (1 · 4) | 5 (2 · 4) | 5 (2 · 3) |
| 6 | Repetition^{a} | 3 | 3 | 3 | 3 | 3 |

^{the} same conditions were used for both the nozzles.
^{b}THF solution of PdCl₂(PhCN)₂ (1.0 mM).
^{c}THF solution of PdCl₂(PhCN)₂ (2.0 mM).
^{d}CH₂Cl₂/MeOH (1:1 v/v) solution of complex 1 (0.2 mM).

### Conclusion

The multi-color electrochromic behavior of coordination-based molecular assemblies (MAs) has been demonstrated. The assemblies were formed in one embodiment by automated spray coating of a mixture of metal-organic complexes. More importantly, these MAs, in one embodiment, have three distinct redox states, which are clearly visible to the eye. Switching between the three distinct states is achieved by application of different electrical potentials (voltage).

In one embodiment, this invention provides a new strategy/design for the formation of multi-color electrochromic devices (ECDs) based on a color mixing concept, using various polypyridyl metal complexes on a single working electrode. The electrochromic performance of the molecular assemblies (MAs) was tested in solution and in laminated devices. The multi-color laminated devices showed fast switching time, long decay time in open circuits and good redox stability for at least 1200 cycles while switching between different states with a color contrast (ΔTₘₐₓ) of up to 55%. Selective control of the electrical potential allows generation of different states that are fully reversible when the applied potential is reversed. All redox states were clearly visible to the eye. Therefore, multi-color ECD's can be produced without the requirement for multiple working electrodes.

### Example 8

### Electrochemical and Electrochromic Behaviors of Surface Confined Single- vs Multi-Component Metallo Organic Assemblies

In this example, it was demonstrated how ion transport in spray-coated metal-organic assemblies can be controlled by systematically varying the structure of their molecular components (single component assemblies), or by mixing more than one complex (forming multi-component assemblies) while keeping the thickness constant. The penetration of ions in single- and multi-component nano-scale films was studied by electrochemical switching and by diffusion coefficient (D_{f}) performances of the assemblies. The increase in penetration of ions is reflected by higher D_{f} values for the redox-active assemblies. The porosity of the films was studied by trapping inorganic NiCl₂ salts inside the assemblies. It is noted that, electrochemical switching or penetration of an ion not only depends on the porosity but also on the internal structure of the assemblies. In addition, for mixed molecular assemblies consisting two types of redox-active metal complexes, selective control of the potential allows the generation of three different states. The transition between the different states is fully reversible when the applied potentials were reversed. These three states are clearly visible to the eye and make these nanoscale assemblies potential candidate for challenging multi-state electrochromic displays based on a single working electrode. The bimolecular systems exhibit three accessible redox states with characteristic absorption bands, corresponding to the three states. It was found that this newly demonstrated system behaving as a colorful multi-electrochromic molecular-switch has robust stability for a large number of redox cycles.

The application of porous materials is well known in separation of gases, heterogeneous catalysis, energy storage and sensing. Similarly, surface-confined porous materials were used to transport small size molecules. Control of electrochemical transport of ions in surface-confined molecular materials is an important feature of the assemblies in view of their applications mentioned above. However, controlling pore structures in these surface confined assemblies remains a challenge.

In the past, the inventors studied electron transport (ET) or permeability properties of coordination-based molecular assemblies and found that these properties are significantly affected by the thickness and roughness of the films. Herein it is shown that ion transport in spray-coated molecular assemblies (MAs) can be controlled by varying the structure of their molecular component in single-component assemblies, or by mixing of these complexes to form multi-component assemblies while keeping the thickness constant. An additional advantage of the mixed multi-component molecular assemblies (MAs) is that they can have multiple redox-active metal centers. This property allows the use of these nanoscale assemblies as multi-state electrochromic devices based on a single working-electrode.

This example demonstrates a simple solution to produce multicomponent electrochromic metallo-organic assemblies formed by solution-based color mixing on a single electrode without the stipulation for multiple conducting electrodes. In this new setup, ion or charge transport properties of single- and multi-component MAs were studied and it was observed that permeability and switching behaviors of these MAs were altered by changing the molecular structure of the assemblies using a solution of single complex or a solution comprising a mixture of complexes.

The 3D-coordination networks (3D molecular assemblies) are formed by alternated spray coating of the following solutions:
a commercially available palladium salt; and
a solution of an electrochromically active divalent polypyridyl single complexes (1 or 4) or equimolar mixture of two complexes (1·4);
   on transparent conduction oxides (TCOs) using a fully automated spray-coating method reported herein (FIG. 2, FIG. 6B).

The single-component assemblies (MA1, and MA4) have shown two-state switching, whereas mixed multi-component assemblies (MA1·4) have three independent states (two well-defined colored states and one colorless state). Switching occurs upon application of different voltages. It was found that these redox changes are well observed by naked eye. All molecular assemblies were characterized by cyclic voltammetry (CV), UV-vis spectroscopy, X-ray photoelectron spectroscopy (XPS), scanning electron microscopy (SEM), and electrochemistry (FIG. 11). The micro-scale SEM and AFM measurements of MA's confirmed that the surface is grainy and homogenously covered. The root-mean-square roughness for 5 μm×5 μm scan areas calculated from AFM imaging is 38-55 nm for MAs (FIG. 13).

The thickness and inner structure of the MAs were determined by milling with 30 keV Ga⁺ focused ion beam (FIB). The thickness of MA was found to be ˜199 nm (MA1), ˜204 nm (MA4) and ˜184 nm (MA1·4) (FIG. 13). XPS analysis of MAs revealed that the Pd content is very close to the expected ratio for a fully formed network, apparently, the MA are expected to be fully cross-linked (FIG. 13).

Three kinds of coordination-based assemblies were prepared to study the electrochemical phenomenon of single component vs mix- or bi-component MAs. All the films were fabricated under the same conditions using automated spray coater to study the effect more clearly. Firstly, the fluorine-doped tin oxide (FTO) glass surfaces were modified by spray-coating a solution of PdCl₂(PhCN)₂ (1 mM, THF) on to the FTO glass surface. Next, 0.2 mM solutions (DCM/MeOH, 1:1 v/v) of polypyridyl complex 1 (osmium) or 4 (iron), or equimolar mixture of complexes 1 and 4 (osmium and iron, 1:1 v/v), were spray-coated using commercially available automated set-up to afford metallo-organic assemblies (MA1, MA4, and MA1·4). Full details of the spray coating are shown in Table 3. The thickness of all of the three films is similar, ˜200 nm. Single component MA1 and MA4 have shown two-state switching (colored and bleached) due to reversible one electron redox reaction, which results in change in the intensity of the metal-to-ligand charge transfer (MLCT) bands of red color MA1 (FIG. 3A-C) and gray color MA4 (FIG. 3E-G). Whereas, the multi-redox response of MA1·4 films on FTO/glass substrate was monitored upon applying the potentials from 0.2-1.8 V (FIG. 3I-K). In state A (red color), both metal ions Fe²⁺ and Os²⁺ are present in +2 oxidation state.

Upon steadily increasing the potential from 0.2 V to 0.8 V, the color of the film changes to gray (state B). In state B: Os²⁺ metal ion oxidized to Os³⁺, at 0.8 V iron metal ion is still present as Fe²⁺. This results in a gray color to the film. Further increase in the potential to 1.8 V resulted in oxidation of Fe²⁺ to Fe³⁺ and the film become colorless (state C) due to oxidation of both metal centers (Os^{2+/3+} and Fe^{2+/3+}). It should be noted that the three states (red-gray-colorless) were fully reversible when the applied potentials were reversed, these color changes are clearly visible to the eye (FIG. 3I). These observations are further supported by UV/vis absorption spectral changes and cyclic voltammetry of [MA1·4|FTO/glass] (FIG. 3J-K). Further, electrochemical behavior of the single (MA1 and MA4) and multicomponent (MA1·4) films was investigated by comparing the switching time of MA1 and MA4 (single component MA) and MA1·4 (bicomponent MA) as shown in FIG. 3D, H and L.

Electrochemical switching studies of all three MA films were carried out to monitor the optical contrast at their absorption maxima and to determine the switching time by stepping the potential between the reduced and oxidized states. The switching time was calculated as being the time taken to reach 90% saturation in the optical transmittance. Single component [MA1|FTO/glass] film formed from 3-arm osmium complex 1, revealed a 0.26 s coloring time and 0.22 s bleaching time when switching between 0.2V and 1.6V at 530 nm (FIG. 3D). Film formed from 6-arm iron complex 4 (MA4) shows a slightly higher coloring time as well as bleaching time (0.8 s/0.4 s) as compared to MA1, while switching between 0.4V and 1.8V at 592 nm (FIG. 3H). However, surprisingly when MA1·4 switched by using double potential step 0.2V to 1.8V at 530 nm between state A-C, response time for MA1·4 found considerably higher (1.6 s/1.8 s) as compared to MA1 and MA4 (FIG. 3L).

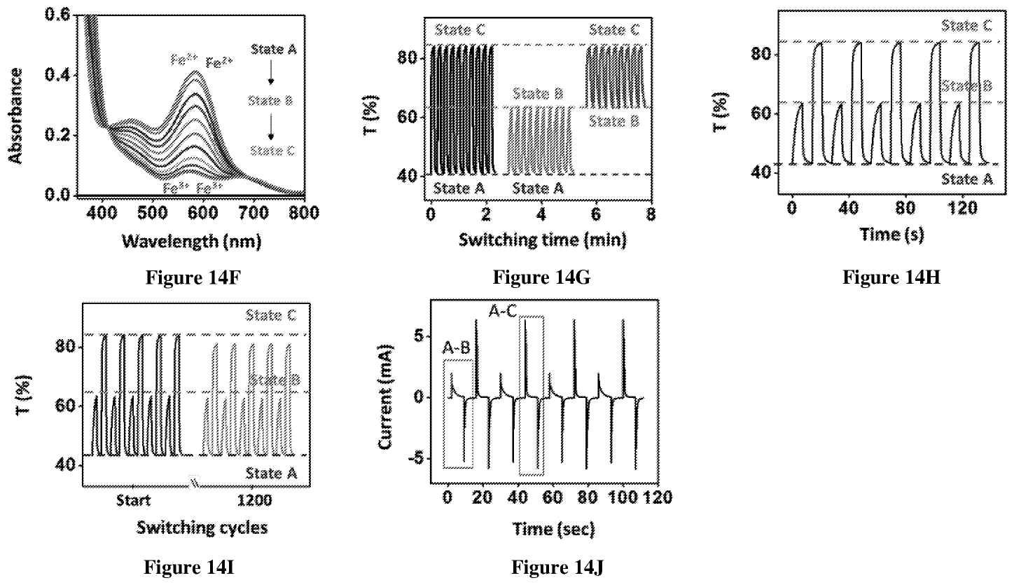

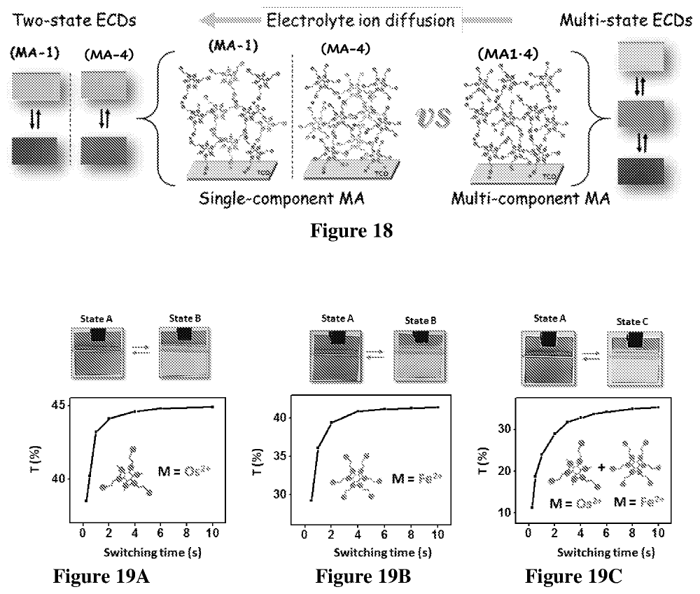

Generally, faster electrochemical response could be influenced by several factors: (1) diffusion of counterion, (2) composition/concentration of electrolyte, and (3) thickness of the film. Factors (2), and (3) could also be omitted because all MA films are tested in the same electrolyte (0.1 M TBAPF6/acetonitrile) using similar ˜200 nm thick films. In this contest, the faster electrochemical switching might be due to faster counterion diffusion (factor: 1), which further depends on channels or pore size in MA films. As electrochemical switching in these MAs had to involve doping or de-doping of electrolyte ions during redox processes to maintain electro-neutrality. It should be noted that switching time of different MAs depends on the type/nature of the metal complex used to form these MAs; therefore, these assemblies should have different pore/ channels size, which further affects the diffusion of ions in these MAs. MA1 formed from complex 1 might have large channels and more free diffusion of ions, resulted in fast response time for MA1 films as compared to MA4 films due to the presence of three-vinylpyridine group of complex 1. The additional three-vinylpyridine groups of complex 4, are likely to form MA with small channels as compared to complex 1, which hamper the diffusion of the counterion (PF6⁻) in MA4 and resulted in long response time. These results are supported by the diffusion coefficient of each film, MA1 (˜1.5×10⁻⁸ cm²s⁻¹) and MA4 (˜1×10⁻⁸ cm²s⁻¹), calculated using to the Randles-Sevcik equation. However, switching time for mixed MA1·4 was found to be even higher than MA4. This is supported by the lowest Df (˜0.8×10⁻⁸ cm²s⁻¹) value for this film (FIG. 21, Table 4). These charge transfer studies were further supported by monitoring the contrast ratio by continuous changes of pulse width for all the three MA1, MA4 and MA1·4 films. The initial contrast ratio (ΔT, %) with 10 s pulse width, of MA1 and MA4 films are 44.9 and 41.4 respectively. The contrast ratio of MA1 film decreased to 44.9, 44.8, 44.6, 44.1, 43.2, and 40.2 (total loss of ΔT=4.7%) when the applied pulse widths shortened to 8, 6, 4, 2, 1 and 0.5 s respectively. On the contrary, the contrast ratio (ΔT, %) of MA4 film decreased to 41.3, 41.2, 40.9, 39.4, 35.9, and 30 (total loss of ΔT=11.4%) with the same applied pulse widths (Table 4). Lesser loss of the contrast ratios of MA1 film indicated that the MA1 film has more rapid counterion diffusion than the MA4 film. On the other hand, the contrast ratio (ΔT, %) of mixed MA1·4 film decreased from initial 36 to 35.7, 34.9, 33.5, 29.7, 24.7, and 18 (total loss of ΔT=18%) with the same applied pulse widths, proved slowest counterion diffusion in MA1·4 film (Table 4). This might be due to smallest channels formed in this case due to tightly-stacked mixture of complexes. Therefore, to understand this in more detail the morphologies of MA1, MA4, and MA1·4 films were evaluated by SEM cross-section images (FIG. 17). The SEM study demonstrated that both films MA1, and MA1·4 are less dense and have more porous morphology as compared to MA4 films (FIG. 17C, F, J). Surprisingly, although MA1·4 film has less dense or stacked morphology as compared to MA4, the switching time for MA1·4 film is longer.

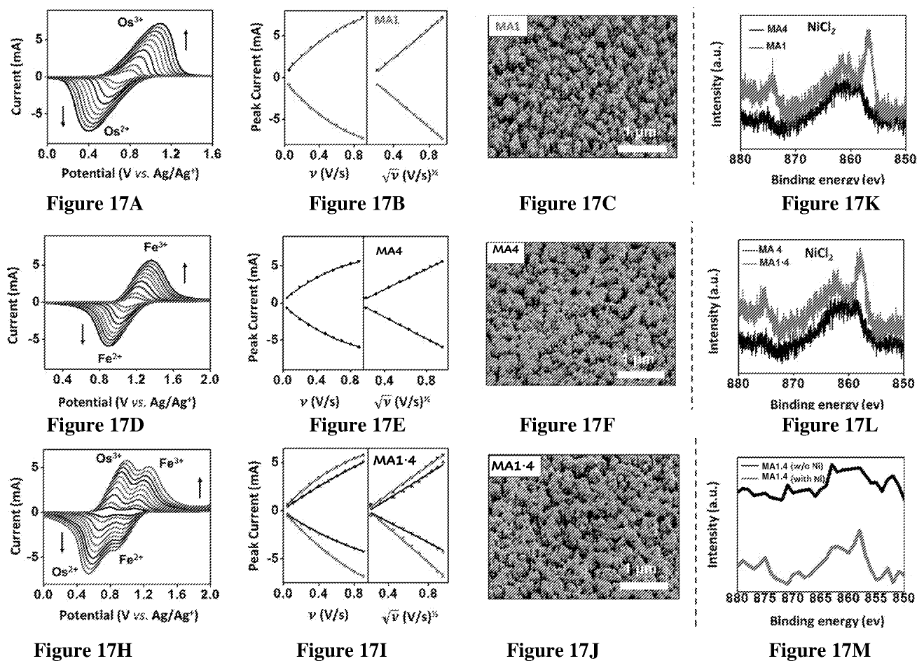

To further understand the porosity or permeability of these films in more detail, trapping of inorganic salts of NiCl₂ inside MA1, MA4 and MA1·4 was investigated (FIG. 20). The FTO-bound assemblies (MA1, MA4 and MA1·4) were immersed for 45 min in 10 mM solutions of NiCl₂.6H₂O in ethanol. Next, substrates were washed with ethanol and subsequently dried. The elemental composition of the films, including the trapped NiCl₂, were analyzed by X-ray photoelectron spectroscopy (XPS). The observed experimental ratios are in very good agreement with the calculated expected values (Table 5). The XPS data shows that the observed nickel content in MA1 are 2.5× higher than MA4, similarly, nickel content in MA1·4 found 2.9× higher than MA4, indicating the high permeability of the MA1 and MA1·4. The observed permeability of these surface-confined MAs supports our findings for switching time difference in case of MA1 and MA4 assemblies. The permeability of MA1·4 is much higher as compared to MA4 and is similar to that of MA1, but still the response time for MA1·4 films is much longer. This might be due to more disordered coordination network formed at the molecular level in the case of mixed metal complexes. This disordered coordination network results in less proper communication between the different centers and results in slow transfer of electron/charge through this disordered architecture. This in turn results in longer switching time of MA1·4 films. Therefore, in this study, it was found that electrochemical switching or penetration of ion/charge not only depends on the porosity but also on the internal structure/architecture of these molecular assemblies. Therefore, it is possible to control the electrochemical properties of these assemblies by using various types of complexes having different types of ligand structures. The mixed molecular assemblies have two redox-active centers, and selective control of potential allows the generation of different states or colors, which are clearly visible to the eyes.

These multi-component assemblies were next tested as multi-state electrochromic displays based on single working electrode. For this purpose, the electrochromic response of these multicomponent MAs was demonstrated for use as a multicolor display. A multi-state laminated electrochromic devices were constructed by using multicomponent MAs on FTO/glass, as working electrode and a thin layer of PEDOT:PSS covered FTO as counter electrode, where PEDOT:PSS layer act as charge storage layer. Both electrodes are separated by a LiClO₄/PMMA based gel-electrolyte and a double-sided tape as spacer (FIG. 11).

Photographs of multi-ECD based on [MA1·4|FTO/glass] film with three redox states (red-gray-colorless) shown in FIG. 11B, red color of the device changes to gray and to colorless under the application of various applied potentials (−1.8 V to +3.0 V) and colors reverse back upon reserving the potential (+3.0 V to −1.8 V). UV/vis measurements clearly show the corresponding reversible changes in the spectral intensities of the MLCT bands corresponding to osmium (530 nm and 703 nm) and iron (592 nm) complexes of the laminated device. The gradual increase in the potential of the laminated device, indicating the complete oxidation of Os^{2+/3+} at +2.0 V and the MLCT band at 530 and 703 nm that disappeared, and oxidation of Fe^{2+/3+} at +3.0 V resulted in the decrease of MLCT band corresponding to 592 nm (FIGS. 11C-11D). Transmittance spectra of the fully reduced state (red colored) and fully oxidized state (bleached) displays highest contrast ratios (ΔT, 43%) at λ=530 nm (FIG. 11E). Further, the transmittances of MA1·4 multi-ECD monitored between state A-C (potential was swiped between −1.8 V to +3V) as a function of different wavelength with 20 s pulse width, and at λ=530 nm; maximum contrast ratio (=43%) (FIG. 11F). These devices have good reversible switching between different states: A-C (−1.8 V to +3V), A-B (−1.8 V to +2 V), and B-C (−0.8 V to +3 V) which shows the utility of these multi-state laminated devices in display industries.

Delightfully, response time for switching these assemblies is ˜3.2 s while switching between state A to C, which is much faster than many of the reported laminated devices (FIG. 11G,H). Next, long stability of these multi-ECD was studied using spectroelectrochemistry (SEC) measurements which show that initial ΔT value (=43%) remains stable for at least 1200 redox cycles without losing noticeable contrast ratio while switching continually between state A to B and A and C at λ=530 nm using double potential steps alternatively: (i) −1.8 V to +2 V for A-B and (ii) −1.8 V to +3 V for A-C (FIG. 11 I,J). The ability to maintain the transmittance value after the applied potential is switched off was also tested for the laminated devices to verify its applicability in information storage. The measured decay time for state B and C, when the applied potential of +2 V and +3 V was turned off, were found to be ˜25 min and ˜90 min, respectively. The observed decay kinetics for state B and C are 0.14 min⁻¹ and 0.06 min⁻¹, respectively (FIG. 11K). These values are higher than reported for many electrochromic metal oxides and some of the best-performing organic polymers.

To summarize, in this example it was demonstrated that coordination based multi-color electrochromic devices using color-mixing concept on single working electrode are practical and effective. The strategy shown in this example was based upon the fabrication of a 3D molecular assembly on TCOs by automatic ultrasonic spray coating equimolar mixture of polypyridyl metal complexes comprising two distinguishable redox couples. These surface-confined MAs exhibit three accessible redox states at relatively different voltages and each state reveals clear characteristic absorption bands in the visible spectra, to give three complementary colors. Delightfully, all these states are clearly visible to the eyes and are very attractive to the field of EC display. Further this approach offers an opportunity to use these systems as electrochromic-molecular switches with multiple logic states on single working electrode.

In addition, we also showed that electrochemical behavior or diffusion of counterions were strongly depending on the chemical structures of the MA films. It was proved that ion or charge transport is not only controlled by porosity of the MAs (MA1 vs. MA4) but also on the mixed internal coordination network formed in multi-component MAs (MA1·4 vs. MA1 and MA4) by mixing of two type of metal complexes.

### Materials and Methods

Solvents (AR-grade) were purchased from Bio-Lab (Jerusalem), Frutarom (Haifa, Israel), or Mallinckrodt Baker (Phillipsburg, N.J.). Poly(methyl methacrylate) (PMMA), lithium perchlorate (LiClO₄), and PdCl₂(PhCN)₂ were purchased from Sigma-Aldrich. Fluorine-doped tin oxide-(FTO)-coated glass substrates (6 cm×6 cm, Rs=8-12Ω/□) and indium-tin oxide (ITO)-coated poly(ethylene terephthalate) (PET) substrates (10 cm×10 cm, Rs=10, 30, 60Ω/□) were purchased from Xinyan Technology Ltd. (Hong Kong, China). FTO-coated glass substrates were cleaned by sonication in ethanol for 10 min, dried under a stream of N₂, and subsequently cleaned for 20 min with UV and ozone in a UVOCS cleaning system (Montgomery, Pa). The substrates were then rinsed with tetrahydrofuran (THF), dried under a stream of N₂, and oven-dried at 130° C. for 2 h. ITO-coated PET substrates were cleaned by immersing for 30 s in ethanol and acetone and then drying under a stream of air.

UV/Vis Spectroscopy. UV/vis spectra were recorded on a Cary 100 spectrophotometer. The absorbance was measured using the Cary Win UV-Scan application program, version 3.00 (182) by Varian (200-800 nm), whereas the transmittance was measured using the Cary Win UV-Kinetics application program, version 3.00 (182) by Varian. Bare substrates were used to compensate for the background absorption.

X-ray Photoelectron Spectroscopy. XPS measurements were carried out on FTO/glass substrates (2.0 cm×2.0 cm) with a Kratos AXIS ULTRA system, using a monochromatic Al Kα X-ray source (hν=1486.6 eV) at 75 W and detection pass energies ranging between 20 and 80 eV. Curve-fitting analysis was based on Shirley or linear background subtraction and application of Gaussi an-Lorenzi an line shapes.

Atomic Force Microscopy (AFM). AFM imaging was carried out by using a JPK AFM (JPK Nanowizard III, Berlin, Germany). Scans of 512×512 pixels per image were made in QI mode using a Quartz like probe (qp-BioAC CB1) with a spring constant of 0.15-0.55 N/m.

Focused Ion Beam (FIB) Microscopy. SEM images were recorded using a Helios 600 FIB/SEM dual-beam microscope (FEI), operating at 5 keV. The images were taken at the surface of the samples and at cross sections that were milled with a 30 keV Ga⁺ focused ion beam (FIB). MA|FTO/Glass 10Ω/□ was first coated with a 3-nm-thick layer of iridium, followed by coating a 150-200 nm-thick layer of platinum using electron-beam-assisted deposition. This process was followed by anion-beam-assisted deposition of a 500-600-nm-thick layer of platinum. The platinum coating protects the MA from ion-beam damage.

Electrochemical Characterization of multi-color electrochromic films. Electrochemical experiments were carried out using a CHI660A or a CHI760E electrochemical workstation. The electrochemical cell consisted of the MA on FTO/glass substrate (1 cm×2 cm or 2 cm×2 cm) serving as the working electrode, Ag/Ag⁺ was used as the quasi-reference electrode, and a Pt wire was used as the counter electrode. Tetrabutylammonium hexafluorophosphate (TBAPF₆) in ACN (0.1 M) was used as the supporting electrolyte.

Formation of molecular assemblies (MAs). Spray coating was performed with an automatic Ultrasonic Spraying System (Sono-tek) equipped with two ultrasonic nozzles having 2 mm −6 mm diameter spray areas, operating at 120 kHz, which were mounted onto an X-Y-Z movable scanner.

Formation of bimolecular assemblies (MA1·4) using a mixture of complexes 1·4. The MA1·4 were obtained by automated ultrasonic spray-coating of solutions of PdCl₂(PhCN)₂ and equimolar mixtures of complexes 1·4, at an atomization pressure of 1.30 kPa. The nozzle-to-substrate distance was 5.5 cm and the nozzle was moved in a pre-programmed pattern along the X and Y direction at a speed of 5 mm/s and with a flow rate of 0.6 mL/min at room temperature (˜23° C.). A THF solution of PdCl₂(PhCN)₂ (1.0 mM) and an equimolar CH₂Cl₂/MeOH (1:1 v/v) solution of complexes 1·4 (0.2 mM each) were used for the formation of the MA1·4. The solution of PdCl₂(PhCN)₂ (1.0 mM) was sprayed onto the substrate (8 passes), which was followed by spraying (5 passes) the mixture of metal complexes solution (0.2 mM). This deposition sequence was repeated 3× to generate the MA1·4. The substrates were then immersed in acetone for 30 s and were dried under a gentle stream of air (Table 3).

Formation of single component molecular assemblies (MA1 or MA4). The MA1 or MA4 were obtained by automated ultrasonic spray-coating of solutions of PdCl₂(PhCN)₂ and complexes 1 or 4, at an atomization pressure of 1.30 kPa. The nozzle-to-substrate distance was 5.5 cm and the nozzle was moved in a pre-programmed pattern along the X and Y direction at a speed of 5 mm/s and with a flow rate of 0.6 mL/min at room temperature (˜23° C.). A THF solution of PdCl₂(PhCN)₂ (1.0 mM) and CH₂Cl₂/MeOH (1:1 v/v) solution of complexes 1 or 4 (0.2 mM each) were used for the formation of the MA1 or MA4. The solution of PdCl₂(PhCN)₂ (1.0 mM) was sprayed onto the substrate (8 passes), which was followed by spraying (5 passes) the mixture of metal complexes solution (0.2 mM). This deposition sequence was repeated 3× to generate the MA1 or MA4. The substrates were then immersed in acetone for 30 s and were dried under a gentle stream of air (Table 3).

Fabrication of laminated multi-color electrochromic devices. A layered architecture was used to construct laminated sandwich cells based on FTO/glass substrates (2 cm×2 cm) coated with MAs serving as working electrodes. PEDOT:PSS-coated FTO/glass substrates (2 cm×2 cm) were used as reference and counter electrodes, respectively. A solution of PEDOT:PSS and isopropyl alcohol (1:1.4 v/v) was drop-casted onto FTO/glass substrates. Subsequently, the substrate was spun at 500 rpm for 10 s and then at 1000 rpm for 30 s. Next, the substrate was heated in an oven at 120° C. for 1 min. A frame of 210-μm-thick double-sided tape (3M 9088) was attached to the working electrode, leaving an exposed edge (1-2 mm) for copper tape contacts. Contacts were also connected to an edge (1-2 mm) of the counter electrode. The two electrodes were placed with the two conducting faces facing each other. The electrolyte gel (90:7:3 wt % ACN/PMMA/lithium perchlorate salt) was injected using a syringe between the two electrodes.

TABLE 3
Spray coating parameters for fabricating electrochromic assemblies
(MA1, MA4, and MA1 · 4) on FTO/glass (2 cm × 2 cm).

| Entry | Spray parameters | MA1^{b, d} | MA4^{c, d} | MA1 · 4^{b, d} |
|---|---|---|---|---|
| 1 | Nozzle to substrate | 5.5 cm | 5.5 cm | 5.5 cm |
| | distance (cm)^{a} | | | |
| 2 | Atomization (kPa)^{a} | 1.30 | 1.30 | 1.30 |
| 3 | Flow rate (mL/min)^{a} | 0.6 | 0.6 | 0.6 |
| 4 | Nozzle speed (mm/s)^{a} | 5 | 5 | 5 |
| 5 | Number of passes | 8 (PdCl₂) | 8 (PdCl₂) | 8 (PdCl₂) |
| | | 5 (1) | 5 (4) | 5 (1 · 4) |
| 6 | Repetition^{a} | 3 | 3 | 3 |

^{a}the same conditions were used for both the nozzles.
^{b}THF solution of PdCl₂(PhCN)₂ (1.0 mM).
^{d}CH₂Cl₂/MeOH (1:1 v/v) solution of complex 1 or 4 or 1 · 4 (0.2 mM).

TABLE 4
Comparison of contrast ratio vs. pulse width vs. diffusion coefficient vs. switching
time of single component [MA1 | FTO/glass], [MA4 | FTO/glass] films
and multicomponent [MA1 · 4 | FTO/glass] in 0.1M TBAPF₆/ACN electrolyte solution.

| | | | | | | | | Loss | Diffusion | Switching time^{b} |
|---|---|---|---|---|---|---|---|---|---|---|
| MA | 10 s | 8 s | 6 s | 4 s | 2 s | 1 s | 0.5 s | of ΔT | coefficient (D_{f})^{a} | (bleaching/colored) |
| MA1 (Os-3) | 44.9 | 44.9 | 44.8 | 44.6 | 44.1 | 43.2 | 40.2 | =4.7 | Ox = 1.5 × 10⁻⁸ | 0.22/0.26 |
| | | | | | | | | | Red = 1.6 × 10⁻⁸ | |
| MA4 (Fe-6) | 41.4 | 41.3 | 41.2 | 40.9 | 39.4 | 35.9 | 30 | =11.4 | Ox = 1 × 10⁻⁸ | 0.8/0.4 |
| | | | | | | | | | Red = 1.1 × 10⁻⁸ | |
| MA1 · 4 | 36 | 35.7 | 34.9 | 33.5 | 29.7 | 24.7 | 18 | =18 | Ox = 0.8 × 10⁻⁸ | 1.6/1.8 |
| (Mixture) | | | | | | | | | Red = 0.6 × 10⁻⁸ | |

^{a}These values were derived from the Randles-Sevcik equation: iₚ = (2.69 × 105)n^{3/2}ACD_{f}^{1/2}v^{1/2}, where iₚ is the peak current (A), n is the number of electrons transferred in the redox reaction, A is the area of the electrode (cm₂), C is the concentration of the solution (mol · cm⁻³), Df is the diffusion coefficient (cm² · s⁻¹), and v is the scan rate (V · s⁻¹).
^{b}Time required in second to change the color to 90% of ΔTₘₐₓ.

TABLE 5
Elemental ratio in NiCl₂ trapped MAs. Various elements
ratio with respect to Ni in MA1, MA4 and MA1 · 4.

| Elements/ | P | F | Cl | Pd | Os | Fe | Fe + | Ni | C | N |
|---|---|---|---|---|---|---|---|---|---|---|
| sample | (%) | (%) | (%) | (%) | (%) | 1%) | Os | (%) | (%) | (%) |
| MA1 (Os) | 3.43 | 12.06 | 50.35 | 21.87 | 10.99 | — | — | 8.51 | 816.7 | 100 |
| MA4 (Fe) | 3.94 | 15.73 | 78.99 | 35.81 | — | 7.44 | — | 3.05 | 774 | 100 |
| MA1 · 4 | 1.31 | 4.92 | 47.08 | 17.62 | — | — | 14.42 | 16.84 | 1287.7 | 100 |
| (mixture) | | | | | | | | | | |
| MA2 (Fe) | 4.99 | 27.47 | 71.14 | 27.28 | — | 11.05 | — | 8.58 | 780.5 | 100 |
| MA4 · 2 | 1.76 | 6.58 | 54.23 | 23.11 | — | 15.32 | — | 15.75 | 777.5 | 100 |
| (mixture) | | | | | | | | | | |

TABLE 6
Comparison of contrast ratio vs pulse width vs diffusion coefficient vs switching
time of single component [MA4 | FTO/glass], [MA4 | FTO/glass]
films and multicomponent [MA4 · 2 | FTO/glass], in a 0.1M TBAPF₆/ACN electrolyte solution.

| | | | | | | | | Loss | Diffusion | Switching time^{b} |
|---|---|---|---|---|---|---|---|---|---|---|
| MA | 10 s | 8 s | 6 s | 4 s | 2 s | 1 s | 0.5 s | of ΔT | coefficient (D_{f})^{a} | (bleaching/colored) |
| MA2 (Fe-3) | 48.7 | 48.4 | 48.0 | 47.4 | 46.8 | 45.7 | 42.1 | =6.6 | Ox = 2.4 × 10⁻⁸ | 0.45/0.30 |
| | | | | | | | | | Red = 2.4 × 10⁻⁸ | |
| MA4 (Fe-6) | 41.4 | 41.3 | 41.2 | 40.9 | 39.4 | 35.9 | 30 | =11.4 | Ox = 1 × 10⁻⁸ | 0.8/0.4 |
| | | | | | | | | | Red = 1.1 × 10⁻⁸ | |
| MA4 · 2 | 41 | 40.6 | 39.8 | 38.5 | 34.6 | 39.1 | 21.8 | =19.2 | Ox = 0.5 × 10⁻⁸ | 1.9/0.9 |
| (Mixture) | | | | | | | | | Red = 0.6 × 10⁻⁸ | |

^{a}These values were derived from the Randles-Sevcik equation: iₚ = (2.69 × 105)n^{3/2}ACD_{f}^{1/2}v^{1/2}, where iₚ is the peak current (A), n is the number of electrons transferred in the redox reaction, A is the area of the electrode (cm₂), C is the concentration of the solution (mol · cm⁻³), Df is the diffusion coefficient (cm² · s⁻¹), and v is the scan rate (V · s⁻¹).
^{b}Time required in second to change the color to 90% of ΔTₘₐₓ.

TABLE 7
Comparison of current approach with previously reported approaches
for the formation of multi-state films on single working electrode.

| Molecular unit | Method | Study | References |
|---|---|---|---|
| bimetallic complex | Monolayer film | Multi-state molecular | Chem. Commun., |
| | of bimetallic | logic | 2014, 50, 3783-3785 |
| | component | | |
| Phthalocyanine double- | Monolayer on single | Multi-state molecular | Chem. Sci., |
| decker complex | working electrode | logic | 2016, 7, 4940-4944 |
| Cyclometalated complex | Single working | Multi-state molecular | J. Am. Chem. Soc. |
| with a redox-active amine | electrode | logic | 2015, 137, 4058-4061. |
| bridge | | | |
| Ruthenium complex and | Tri-layer film on | Charge trapping and | J. Am. Chem. Soc. |
| Prussian blue (PB) | single working | release | 2014, 136, 842-845 |
| nanoparticles | electrode | | |
| Metal-organic polypyridyl | Sequence-dependent | Charge trapping and | (a) Angew. Chem. Int. Ed. |
| complexes^{a} | molecular assemblies | electrochemical | 2013, 52, 704-709. |
| | on single working | communication | (b) J. Am. Chem. Soc. |
| | electrode | between the metal | 2013, 135, 16533-16544. |
| | | centers | |
| Metal-organic polypyridyl | Sequence-dependent | Charge trapping and | Angew. Chem. Int. Ed. |
| complexes^{a} | molecular assemblies | releasing coupled | 2019, |
| | on single working | with electrochromic | |
| | electrode | | |
| Metal-organic polypyridyl | Multi-component | Electrochemical and | This work |
| complexes | molecular assemblies | electrochromic study | |
| | on single working | of single vs multi- | |
| | electrode | component MAs | |

^{a}Inventor's earlier reports.

TABLE 8
Comparison of multi-state electrochromic properties of current material with previously
reported different type materials based on a single working electrode.

| Molecular unit | Optical contrast | Laminated | Switching stability | |
|---|---|---|---|---|
| or binding ligand | (ΔTₘₐₓ, %) | devices | of laminated devices | References |
| Conjugated polymers | up to 42 | not reported | not reported | J. Mater. Chem., |
| | | | | 2011, 21, 1804-1809 |
| Monoheptyl and diheptyl | up to 25 | not reported | not reported | ACS Appl. Mater. Interfaces |
| viologen based Ion gels | | | | 2017, 9, 7658-7665 |
| Plasmonics polycyclic | up to 79 | shown | up to 100 cycles | ACS Nano |
| aromatic hydrocarbon | | | | 2017, 11, 3254-3261 |
| Au/Ag alloy nanoparticles | not reported | not reported | not reported | Small |
| | | | | 2019, 15, 1804974 |
| Bimetallic metallo- | up to 52.8 | shown | up to 200 cycles | J. Mater. Chem. C, |
| supramolecular polymer | | | | 2019, 7, 7554-7562 |
| based on terpyridine | | | | |
| ligands | | | | |
| Metallo-supramolecular | up to 40 | shown | not reported | ACS Appl. Mater. Interfaces, |
| polymer based on | | | | 2015, 7, 25069-25076 |
| bis(2,2′:6′,2″-terpyridine) | | | | |
| ligands | | | | |
| Biscyclometalated complex | up to 40 | not reported | not reported | J. Am. Chem. Soc. |
| | | | | 2011, 133, 20720-20723. |
| Metal-organic polypyridyl | Not shown | shown | up to 20 cycles | Angew. Chem. Int. Ed. |
| complexes^{a} | | | | 2019, |
| Metal-organic molecular | up to 57 | shown | up to 1200 cycles | This work |
| assemblies based on | | | | |
| polypyridyl complexes | | | | |

^{a}Inventor's earlier reports.

### Example 9

### Thermally Stable Electrochromic Metallo-Organic Display Using Photocurable Solid Polymer Electrolyte For Improved Stability, of Devices Without Ion Storage Layer

In this example, the performance of surface-confined molecular assemblies as electrochromic displays (ECDs) was demonstrated. The displays included ultraviolet (UV)-crosslinked polymer network as solvent free solid polymer electrolyte (SPE)-matrix. The layered display can be described as follows: (glass/TCO//EC+solid electrolyte//TCO/glass). The solvent-free electrolyte used, improved the performance of the ECDs without the need for coating the counter electrode with any ion storage layer. This improvement is probably due to controlling the side reactions undergoing in a liquid-gel type electrolyte. These devices can be operated for 4500 redox cycles without losing intense color in the ground state. Importantly, solid-state electrolyte-based devices were stable even at ˜100° C. with retention of device color and switching properties.

Furthermore, the utility of this set up in multi-component assemblies to form multicolor-display was demonstrated. This route offers future application of these assemblies as electrochromic displays.

In some embodiments, it was found that the stability of the MAs in a laminated set-up was improved when the counter electrode was coated with a thin layer of conducting poly(3,4-ethylene-dioxythiophene)polystyrene sulfonate (PEDOT:PSS) as an ion-storage in a liquid gel type electrolyte.

Despite extensive research for over 3-4 decades, only few electrochromic products are commercially available, mainly due to the long response times and complex multi-layer electrochromic device (ECD) structure in liquid-gel type electrolyte. Current devices usually suffer poor stability due to many side reactions occurring in the volatile liquid gel type electrolyte. Further, the organic-solvent based electrolytes are flammable or toxic and have leakage problems resulting in safety concerns. These limitations prevent development of the technology for wide display applications.

To overcome these drawbacks and make these materials industrially favourable, there is a continuous effort focused on replacing organic liquid electrolytes with solid-state electrolyte possessing high ionic conductivity. Over the past few years, electronic devices have been prepared using photocurable solid-gel polymer electrolytes including batteries, transistors and ECDs. Reported ECDs, mostly based on conducting polymers and viologen derivatives embedded within the electrolyte are detailed in Table 10. One reported device was based on an electrochromic polymer blend formed by dissolving monomer in UV active liquid electrolyte and assembled it into a device, followed by exposure to UV light to generate a solid electrolyte matrix. This method is limited to those EC monomers which can be dissolved in liquid electrolyte. Similarly, various viologen derivatives dissolved in UV active liquid electrolyte and sandwiched between two electrodes to generate a solid matrix based display with viologens as ECMs has been reported. Another class of solid-state electrolytes was formed by dissolving ECMs (viologen derivatives) with copolymer (poly(vinylidene fluoride-co-hexafluoropropylene) (P(VDF-co-HFP)) and ionic liquids. These ECDs were fabricated by sandwiching the EC gel between two-electrodes using cut-and-stick strategy.

The previously-reported solid polymer electrolytes (SPEs) based ECDs have long switching time and less redox stability (Table 10) which might be attributed to low ionic conductivities created by huge interfacial resistance at the electrode or electrolyte interface and poor contact with the electrodes. This result limits their application. In order to advance the applications of these materials in SPE based set up, it is necessary to improve the combinations of working electrodes and electrolytes to form a structurally simplified and highly stable ECDs.

In this example, a new photocurable crosslinked solid electrolyte was prepared with good long-term stability at up to 100° C. The solid electrolyte is compatible with coordination based molecular assemblies (MAs) in laminated set-up. The stability of the MA-based devices in the SPE set up is much higher than devices reported in liquid electrolyte, without affecting the response time. The ECDs were fabricated using spray-coated MAs (MA1, MA4, and MA1·4) as working electrode and FTO/glass as counter electrode. This approach structurally simplifies device architecture with the absence of ion storage coated counter electrode used in other embodiments to improve stability of these MAs based devices. UV-crosslinked solid polymer matrix was formed by in-situ UV-curing of photocurable liquid gel electrolyte mixture (diacrylate, omnirad-184, LiClO₄, PMMA in ACN/PC). These [MA/solid electrolyte] based devices are thermally stable (˜100° C.), have high redox stability (>4500 cycles) and fast switching time (˜1 s).

As noted above, this example demonstrates a new facile strategy to develop liquid electrolyte free laminated set-up with spray-coated MAs (MAL MA4, and MA1·4) as working electrode, in ultraviolet (UV)-cured diacrylate based cross-linked solid polymer electrolyte. UV-crosslinked polymer networks were formed by in-situ UV-curing of photocurable liquid gel electrolyte mixture (diacrylate, omnirad-184, LiClO₄, PMMA in ACN/PC). These [MA/solid electrolyte] based devices are thermally stable (˜100° C.), have high redox stability (>4500 cycles), and fast switching time (˜1 s). The stability of these devices in SPE set up is much higher than devices reported in liquid electrolyte without affecting the response time. Further, this approach structurally simplifies device architecture with the absence of ion storage coated counter electrode, used in other embodiments to improve stability of these MAs based devices. The MAs are formed by automated spray-coating on FTO/glass substrates using polypyridyl complexes and characterized by optical UV-vis spectroscopy, X-ray photoelectron spectroscopy (XPS), and electrochemistry. The thickness of MM, MA4, and MA1·4 were found to be ˜190, 438, and 184 nm, respectively, with large voids (similar to previously reported spray-coated MAs).

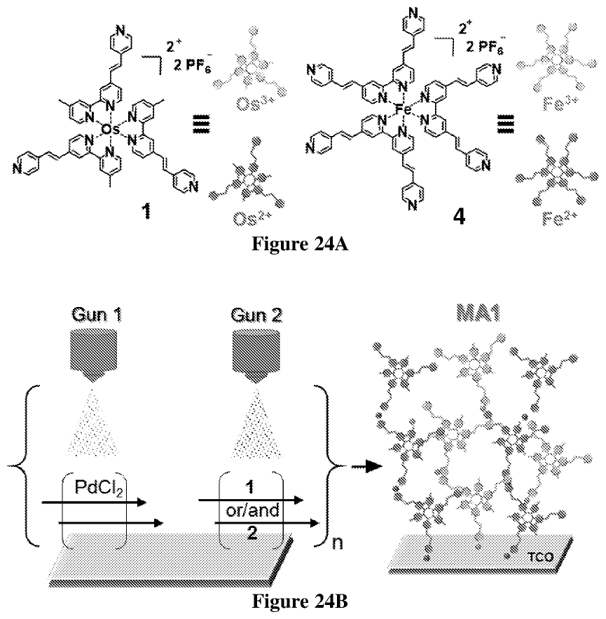

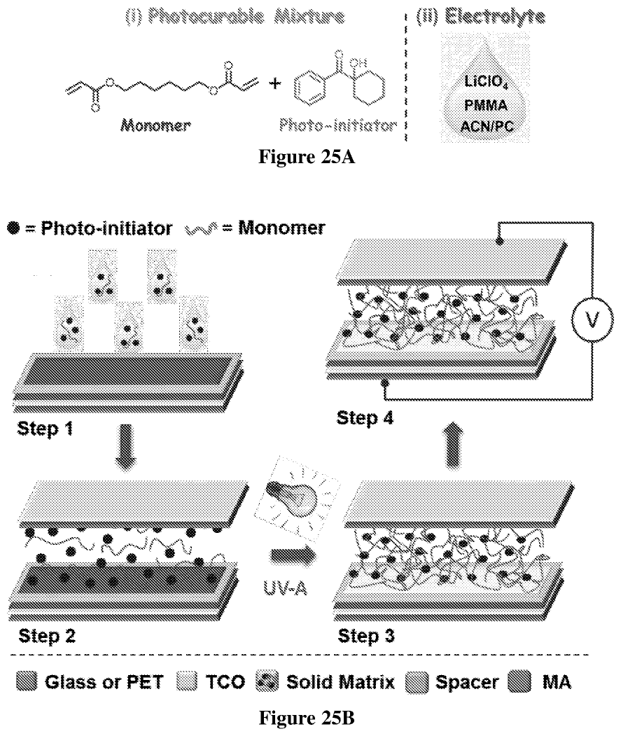

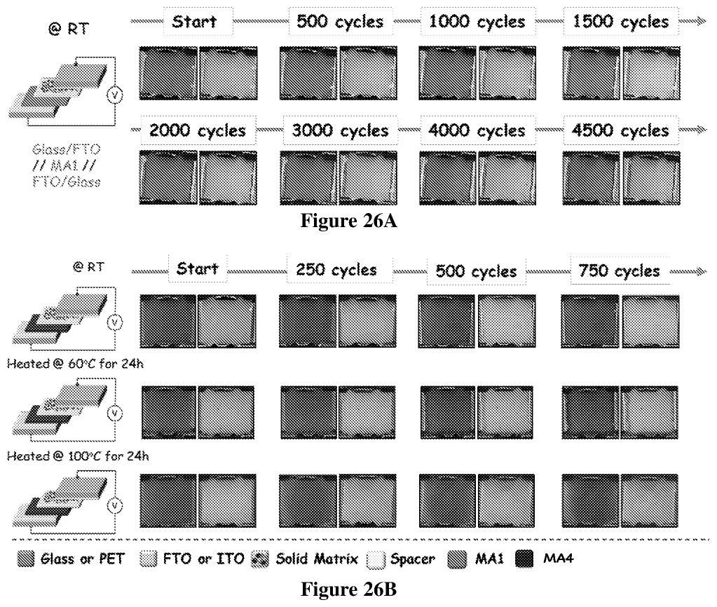

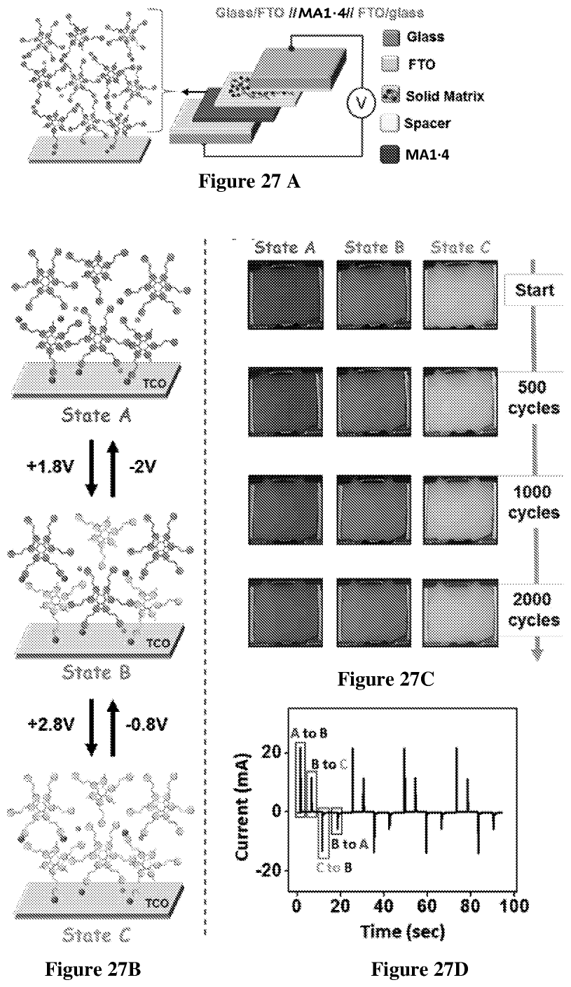

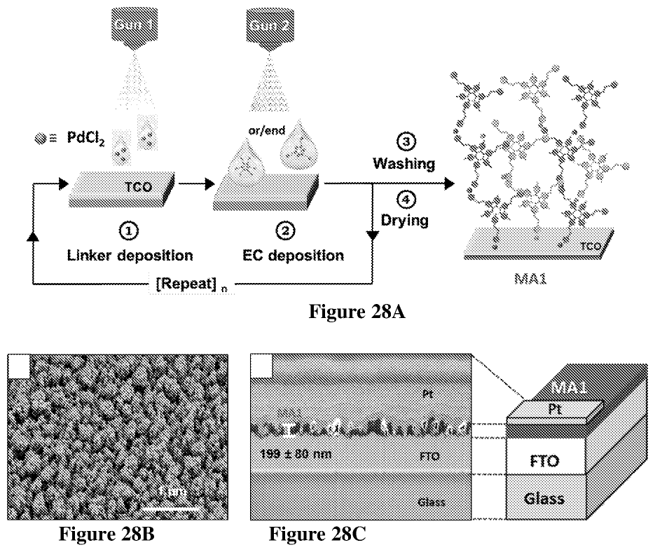

In situ polymerization of liquid monomer electrolyte to form solid electrolyte based electrochromic cell was completed as follow: (a) Step 1: UV-active liquid monomer electrolyte (HDODA (monomer), omnirad 184-resin (photo-initiator), LiClO₄, PMMA, and ACN/PC), was drop casted on working electrode (MAs//FTO/glass) covered with double sided tape as spacer. (b) Step 2: counter electrode (FTO/glass) was placed on the top of working electrode and liquid electrolyte was sandwiched between two electrodes (separated by 210 μm spacer). It should be noted that the electrolyte thickness has major role on the performance of these ECDs, and optimized condition were obtained after series of experiments performed with SPE. (c) Step 3: The laminated device was then placed inside a UV crosslinker to cure the monomer gel electrolyte under UV-A light (365 nm) for 1 min to convert the monomer to a cross-linked polymer network. The device was then connected to a potentiostat, and the electrochromic properties were investigated (FIG. 25). The electrochromic performance of these assemblies was demonstrated using a laminated set up of [MA1|FTO/glass], [MA4|FTO/glass], and [MA1·4|FTO/glass] as working electrodes and FTO/glass as counter electrode. These two electrodes are separated by a solid-state polymer electrolyte matrix (HDODA, omnirad-184, LiClO₄, PMMA, and ACN/PC) and 210 μm thick double-sided tape as spacer (FIGS. 26 and 27). Photographs of the gel electrolyte before and after UV cured light (solid matrix) are shown in FIGS. 28 and 29.

First, electrochromic performance of [MA1|FTO/glass] was investigated using solid-state matrix electrolyte. Oxidation and reduction reaction of osmium based device (active area of the device 1.7 cm×1.3 cm) at an applied potential −2V to +2.8V with a pulse width of 3 s, results in color-to-color switching (red to light yellow) as shown in FIG. 26A. Interestingly, response time for MA1 based device is ˜1 s, which is much faster than many of the reported devices with solid-state electrolytes. [MA1|FTO/glass] based ECDs were switched for at least 4500 redox cycles without losing intense color in the ground state, photographs are shown in FIG. 26A.

Next, electrochromic performance of [MA4|FTO/glass] with active area of the device 1.7 cm×1.3 cm without any ion storage layer on counter electrode, was tested using potential steps of −1.8 to +2.8 V (FIG. 26B, top). The photograph of ECDs showed the consistency of color intensity even after 750 redox switching cycles. (FIG. 26B, top).

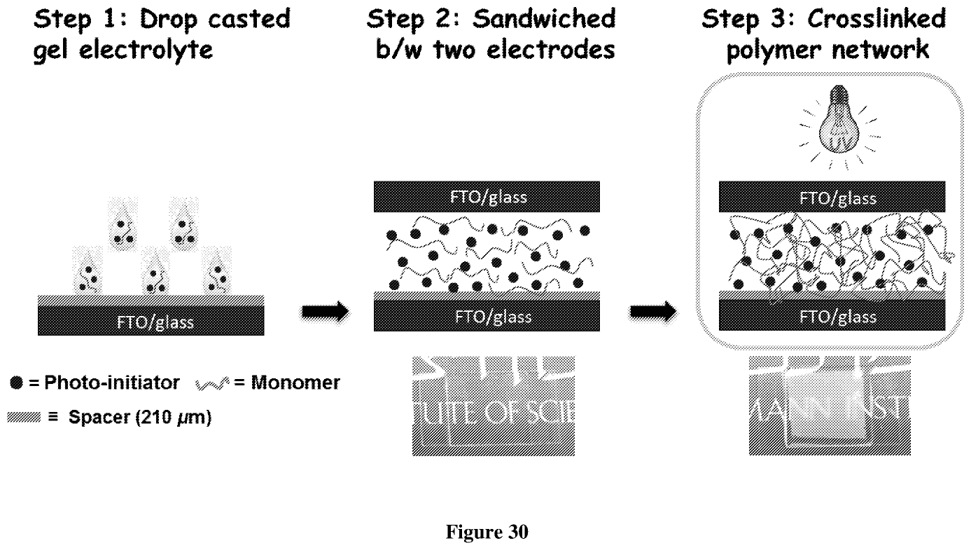

The stability of laminated electrochromic devices at high temperature is an important issue for the use of these devices under harsh conditions. Liquid gel-electrolyte based devices sustainability or stability decreases at high temperature due to dryness of the electrolyte due to evaporation of solvents, and result in low ionic mobility through the laminated device. Therefore, solid polymer electrolyte can provide a better alternative to a liquid gel-based electrolyte. To check the thermal stability of the fabricated devices, electrochromic switching was performed with [MA4|FTO/glass] films based devices using double potential steps: −2 to +3.2 V: first few cycles were run at room temperature (25° C.), followed by heating the device at 60° C. and 100° C. for 24 h. Photographs are shown in FIG. 31. After 24 h heating, the same device was subjected to another ˜750 redox cycles at room temperature to check the color/switching stability of these thermally treated devices (FIG. 26B, middle to bottom). It can be observed from the photographs that the color stability and electrochromic switching was retained even after 750 cycles. Therefore, these results proved the thermal sustainability behaviour of the solid polymer electrolyte and the ability to use this polymer-electrolyte based ECDs set up under extremely hot summer conditions with temperatures of up to 100° C. without compromising performance of the devices.

To further expand the scope of this example, the electrochromic behaviour of multicomponent MA's was demonstrated, in view of their use as multi-color displays. Solid-state-matrix based multi-color laminated electrochromic devices were constructed by using [MA1·4|FTO/glass], as working electrode and FTO/glass as counter electrode which were separated by a spacer (210 μm) and solid electrolyte. (FIG. 27A). MA1·4 assemblies are formed by spray coating the equimolar mixture of polypyridyl complexes 1 and 4 (osmium and iron) using the commercially available automated set-up, on conducting oxides (TCOs) surfaces to afford 3-D network of multi-component assemblies (Table 3). The multi-electrochromic response of MA1·4 films on FTO/glass substrate was monitored upon applying the potentials of from −2V to +2.8 V (FIGS. 27B-27D). In state A, both metal ions Fe²⁺ and Os²⁺ are present in +2 oxidation state resulting in red color display (FIG. 27B). At an applied potential of +2V, the color of the display changed to gray (state B), due to selective oxidation of the osmium center of MA1·4. The iron center remained in the ⁺² oxidation state. Further increase of the potential to +2.8 V resulted in oxidation of the iron center also to give a colorless state C. It should be noted that three states (red-gray-colorless) were fully reversible when the applied potentials were reversed. At applied potential: −0.8 V (gray color, state B), and at −2V (red color, state A) as shown in FIG. 27B. Photographs and chronoamperometric (CA) measurements of multi-color display based on [MA1·4|FTO/glass] film with three redox states: state A=red, state B=gray, and state C=colorless (FIG. 27C). The long-term stability for the UV-cured MA1·4-ECDs was investigated by continuously stepping between these three states using potential +2V, +2.8V for the oxidation process, and −0.8V, −2V for the reduction process. No significant decay in color and switching behavior was observed even after 2000 continuous redox switching as shown in FIG. 27C.

In summary, this example demonstrated electrochromic performance of metallo-organic films in a laminated set-up with solid polymer electrolyte (SPE) formed through a simple in-situ UV-curing of acrylate-based polymer gel electrolyte. Incorporation of the UV-cured electrolyte in these ECDs not only eliminates the leakage and evaporation problem of MA based laminated ECDs, but also significantly improves the color stability of [MA4//FTO/glass] based nanoscale assemblies, without coating the counter electrode with any ion storage layer. This newly reported SPE for metallo-organic assemblies eliminates the need of extra conducting ion storage layer (PEDOT:PSS) coated on the counter electrode which is generally required for improving the performance of these MAs.

Perhaps surprisingly the switching speed for the solid polymer electrolyte based ECDs are similar as for the liquid gel electrolyte type devices. [MA1//FTO/glass] devices can be switched for more than 4500 redox cycles without degradation in switching behavior with excellent response time of ˜1 s. In this example, it was also shown that these SPE based devices are thermally robust and can be stable even after heating up to 100° C. Their working function and device structure are compatible with operation under extremely hot summer conditions. Furthermore, multi-component MAs (containing two redox active species) were successfully demonstrated as multi-color displays with variable color, including dark red, gray and transparent with excellent cycling stability.

### Materials and Methods

Solvents (AR-grade) were purchased from Bio-Lab (Jerusalem), Frutarom (Haifa, Israel), or Mallinckrodt Baker (Phillipsburg, N.J.). Poly(methyl methacrylate) (PMMA), lithium perchlorate (LiClO₄), and PdCl₂(PhCN)₂ were purchased from Sigma-Aldrich and 1,6-Hexanediol diacrylate (HDODA) and Omnirad 184 Resin were purchased from Alfa Aesar and IGM resins. Fluorine-doped tin oxide-(FTO)-coated glass substrates (6 cm×6 cm, Rs=8-12Ω/□) and indium-tin oxide (ITO)-coated poly(ethylene terephthalate) (PET) substrates (10 cm×10 cm, Rs =10, 30, 60Ω/□) were purchased from Xinyan Technology Ltd. (Hong Kong, China). FTO-coated glass substrates were cleaned by sonication in ethanol for 10 min, dried under a stream of N₂, and subsequently cleaned for 20 min with UV and ozone in a UVOCS cleaning system (Montgomery, Pa.). The substrates were then rinsed with tetrahydrofuran (THF), dried under a stream of N₂, and oven-dried at 130° C. for 2 h. ITO-coated PET substrates were cleaned by immersing for 30 sin ethanol and acetone and then drying under a stream of air.

For UV/Vis Spectroscopy, X-ray Photoelectron Spectroscopy, Atomic Force Microscopy (AFM), Focused Ion Beam (FIB) Microscopy and Formation of molecular assemblies (MAs), see Example 8 herein above.

Formation of molecular assemblies (MA1 and 1·4). The MA1 and MA1·4 were obtained by automated ultrasonic spray-coating of solutions of PdCl₂(PhCN)₂ and CH₂Cl₂/MeOH solution of complex 1 (MA1) and equimolar mixtures of complexes 1·4 (MA1·4), respectively, at an atomization pressure of 1.30 kPa. The nozzle-to-substrate distance was 5.5 cm and the nozzle was moved in a pre-programmed pattern along the X and Y direction at a speed of 5 mm/s and with a flow rate of 0.6 mL/min at room temperature (˜23° C.). A THF solution of PdCl₂(PhCN)₂ (1.0 mM) and an equimolar CH₂Cl₂/MeOH (1:1 v/v) solution of complexes 1 or of 1·4 (0.2 mM each) were used for the formation of the MA1 and MA1·4. The solution of PdCl₂(PhCN)₂ (1.0 mM) was sprayed onto the substrate (10 passes), which was followed by spraying (5 passes) the metal complexes solution (0.2 mM) for MA1. The solution of PdCl₂(PhCN)₂ (1.0 mM) was sprayed onto the substrate (8 passes), which was followed by spraying (5 passes) the metal complexes solution (0.2 mM) for MA1·4. This deposition sequence was repeated 3× to generate the MA1 and MA1·4. The substrates were then immersed in acetone for 30 s and were dried under a gentle stream of air (see Table 3).

Preparation of gel monomer electrolyte. Polymethylmethacrylate (PMMA; 40 mg), 50 mg of LiClO₄, 325 μL of UV active monomer 1,6-hexanediol diacrylate (HDODA) and 16 mg of photo-initiator omnirad-184 were added together in 1 mL propylene carbonate/acetonitrile (PC/ACN, 1:1) solution and stirred overnight in dark. The electrolyte is a colorless gel liquid before UV exposure.¹⁻⁴

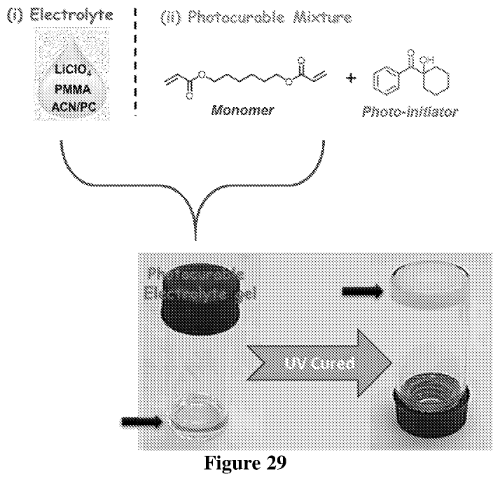

Fabrication of laminated electrochromic display in ambient air. A layered architecture was used to construct laminated sandwich cells based on FTO/glass substrates or ITO/PET (2 cm×2 cm) coated with MAs serving as working electrodes. FTO/glass substrates or ITO/PET (2 cm×2 cm) were used as reference and counter electrodes, respectively. A spacer of 210-μm-thick double-sided tape (3M 9088) was attached to the working electrode, leaving an exposed edge (1-2 mm) for copper tape contacts. First, a solution of liquid monomer electrolyte (ACN/PC/PMMA/lithium perchlorate salt/HDODA/omnirad-184) was drop-casted onto working electrode. Subsequently, the counter electrode was placed on MA-coated FTO/glass or ITO/PET with the gel electrolyte sandwiched between these substrates, which were held tight with an insulating two-sided, 210-μm thick double-sided tape (3M 9088) at each end. The device was then placed in inside a UV cross-linker to cure the gel monomer electrolyte under 365 nm UV light for 1 min, which gave white solid-state matrix. An example of solid-state matrix sandwiched between two FTO/glass substrate shown in FIG. 30.

TABLE 9
Screening of different condition for application of the electrolyte composition before
exposure to UV light, using [MA4|FTO/glass] based film as working electrode.

| S. | | | Switching time | Stability |
|---|---|---|---|---|
| No. | Condition | Result | (sec) | cycles |
| 1 | Spin coated on CE (1 layer), and placed on WE, | No switching | — | — |
| | used immediately or after 12 h standing | | | |
| 2 | Spin coated on CE (2 layer) and placed on WE, | Not complete | 40 | ~20 |
| | used immediately or after 12 h standing | switching | | |
| 3 | Spin coated on WE (2 layer) and placed on CE, | Not complete | 35-40 | ~22 |
| | used immediately or after 12 h standing | switching | | |
| 3 | Drop casted and sandwiched between WE and | Fully | 10 | ~40 |
| | CE, and used immediately (w/o spacer) | Switching | | |
| 4 | Drop casted and sandwiched between WE and | Fully | 10 | ~50 |
| | CE, and used after 12 h standing (w/o spacer) | Switching | | |
| 5 | Drop casted and sandwiched between WE and | Fully | 10 | >750 |
| | CE, and used immediately or after 12 h standing | Switching | | |
| | (with spacer 210 μm) | | | |

TABLE 10
Comparison of reported electrochromic device performance using solid-state or ion-gel electrolyte.

| Type of | Type of | Solid state | Stability | Thermal | |
|---|---|---|---|---|---|
| material | ECDs | electrolyte type | (Cycles) | stability | Reference |
| Viologen | Ion gel: | A homogeneous solution of EC material, | Not shown | Not shown | ACS Appl. Mater. Interfaces |
| derivatives | glass/ITO/Ion | poly(vinylidene fluoride-co- | | | 2016, 8, 6252-6260 |
| | gel//ITC)/glass | hexafluoropropylene) | | | |
| | | (P(VDF-co-HFP)) and ionic liquids such as | | | |
| | | 1-butyl-3-methylimidazolium | | | |
| | | bis(trifluoromethylsulfonyl)imide | | | |
| | | ([BMI][TFSI]) or 1-butyl-3-methylimidazolium | | | |
| | | tetrafluoroborate ([BMI][BF4]). | | | |
| Viologen | Thermally cross- | A homogeneous solution of EC material, Poly(1- | 10,000 | Not shown | (i) ACS Appl. Mater. Interfaces |
| based ionic | linked matrix of | Allyl-3-methylimidazolium | | | 2016, 8, 4175-4184 |
| liquids | EC ion gel: | bis(trifluoromethylsulfonyl)imide), | | | (ii) ACS Appl. Mater. Interfaces |
| | glass/ITO/Ion | Poly[AMIM][TFSI]), | | | 2016, 8, 30351-30361 |
| | gel//ITO/glass | ethoxylated trimethylolpropane triacrylate | | | |
| | | (ETPTA) and ferrocene | | | |
| Viologen | Thermally cross- | A solution of 2,2,6,6-tetramethyl-1- | Not shown | Not shown | Sol. Energy Mater. Sol. Cells, |
| derivatives | linked matrix of | piperidinyloxy (TEMPO) as the anodic radical | | | 2012, 135-140 |
| | EC ion gel: | provider (ion-storage layer), crystals of | | | |
| | glass/ITO/Ion | succinonitrile (SN), and silicon dioxide (SiO₂) | | | |
| | gel//ITO/glass | nanoparticles | | | |
| Viologen | UV cured EC ion | A homogeneous solution of EC material, 1,1- | 1000 | Not shown | Adv. Funct. Mater. |
| derivatives | gel; | dimethyl ferrocene | | | 2019, 29, 1808911 |
| | glass/ITO/Ion | (DMFc), polyethylene glycol) diacrylate | | | |
| | gel//ITO/glass | (PEGDA), 2-hydroxy-2-methylpropiophenone | | | |
| | | (HOMPP), and ionic liquid ([EMIM] | | | |
| | | [TFSI]) | | | |
| Conducting | UV cured Slot- | A solution of polyethylenglycol diacrylate in | 1000 | Not shown | Adv. Mater. |
| Polymers | die coated | ionic liquid 1-ethyl-3-methyl imidazolium | | | 2014, 26, 7231-7234 |
| | electrolyte | bis(trifluoromethane sulfonyl)imide was slot-die | | | |
| | on working | coated on working electrode | | | |
| | electrode: | | | | |
| | grid electrode/ | | | | |
| | PEDOT:PSS/ | | | | |
| | ECM/solid | | | | |
| | electrolyte// | | | | |
| | PEDOT:PSS/ | | | | |
| | grid electrode | | | | |
| Conducting | UV cured EC | A homogeneous solution of EC material, | Not shown | Not shown | (i) J. Mater. Chem. C, |
| Polymers | ion gel: | Propylene carbonate (PC), poly(ethylene glycol) | | | 2014, 2, 2510. |
| | glass/ITO/Ion | diacrylate (PEG-DA), lithium | | | (ii) J. Mater. Chem., |
| | gel//ITO/glass | trifluoromethanesulfonate (LITRIF), 2,2- | | | 2011, 21, 11873-11878. |
| | | dimethoxy-2-phenyl-acetophenone (DMPAP) | | | |
| | | and 1-butyl-3-methylimidazolium | | | |
| | | hexafluorophosphate | | | |
| Metal-organic | UV cured gel | A homogeneous solution of | 4500 | Up to 100° C. | This work |
| molecular | electrolyte: | Polymethylmethacrylate (PMMA), LiClO₄,1,6- | | | |
| assemblies | glass/FTO// | hexanediol diacrylate (HDODA), and omnirad- | | | |
| | ECM//solid | 184 were added together in 1 mL propylene | | | |
| | matrix// | carbonate/acetonitrile (PC/ACN, 1:1) | | | |
| | FTO/glass | | | | |

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A method of preparation of an electrochromic device, said method comprising:
a. providing a substrate;
b. applying a linker comprising a metal ion to said substrate by spray-coating, thus forming a linker layer on said substrate;
c. applying a metal-coordinated organic complex. to said linker layer by spray coating, thus forming a layer of metal-coordinated organic complex on said linker layer;
d. optionally repeating steps b and c;
thereby forming an electrochromic device comprising a substrate and comprising at least one layer of a linker and at least one layer of a metal-coordinated organic complex, wherein the spray coating steps for applying the metal linker and the organic complex are conducted at atomization pressure ranging between 0.75 kPa and 1.50 kPa and at a nozzle to substrate distance ranging between 3.0 and 8.0 cm.

2. The method of claim 1, wherein said metal-coordinated organic complex comprises at least one functional group, said functional group capable of binding to said metal ion, and wherein said binding comprises a coordination bond between said functional group and said metal ion.

3. (canceled)

4. The method of claim 1, wherein said metal-coordinated organic complex is polypyridyl complex.

5. The method of claim 1, wherein the spray coating steps for applying the metal linker and the organic complex are conducted at a spraying solution flow rate ranging between 0.4 and 0.8 mL/min and at room temperature, and wherein said spraying is conducted such that the spraying nozzle is moved parallel to the substrate in a pattern along the X-Y substrate directions at a speed ranging between 3 and 7 mm/s.

6. (canceled)

7. The method of claim 1, wherein following application of the linker layer, following application of the metal-coordinated organic complex layer or a combination thereof, a washing step is conducted for washing the linker layer, for washing the complex layer or a combination thereof, and wherein the washing solvent is selected from the group consisting of alcohols, ethers, esters, halogenated solvents, hydrocarbons, ketones, or a mixture thereof.

8. The method of claim 1, wherein following application of the linker layer, following application of the metal-coordinated organic complex layer or a combination thereof, a drying step is conducted for drying the linker layer, for drying the complex layer or a combination thereof.

9. (canceled)

10. The method of claim 1, wherein both applying steps are repeated to obtain from 2 to 80 linker/organic-complex layers.

11. The method of claim 1, wherein the metal ion in the linker is selected from the group consisting of Pd, Zn, Os, Ru, Fe, Pt, Ni, Ir, Rh, Co, Cu, Re, Tc, Mn, V Nb, Ta, Hf, Zr, Cr, Mo, W, Ti, Sc, Ag, Au, and Y.

12. The method of claim 4, wherein the polypyridyl complex is represented by Formula I: 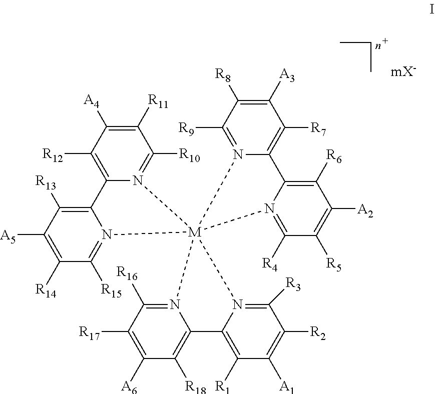
wherein
M is a transition metal selected from Fe, Os, Ru, Co, Ni, Mn, Cu, Zn, Ti, Cr, Rh, or Ir;
n is the formal oxidation state of the transition metal, wherein n is 0-6;
X is a counter ion;
m is a number ranging from 0 to 6;
R₁ to R18 each independently is selected from H, halogen, —OH, —N₃, —NO₂, —CN, —N(R₂₀)₂, —CON(R₂₀)₂, —COOR₂₀, —SR₂₀, —SO₃H, —CH═CH-pyridyl, —(C₁-C₁₀)alkyl, —(C₂-C₁₀)alkenyl, —(C₂-C₁₀)alkynyl, —(C₁-C₁₀)alkoxy, cycloalkyl, heterocycloalkyl, aryl, heteroaryl, wherein the (C₁-C₁₀)alkyl, (C₂-C₁₀)alkenyl, (C₂-C₁₀)alkynyl, cycloalkyl, heterocycloalkyl, aryl, or heteroaryl may optionally be substituted with halogen, —OR₂₀, —COR₂₀, —COOR₂₀, —OCOOR₂₀, —OCON(R₂₀)₂, —(C₁-C₈)alkylene-COOR₂₀, —CN, —N(R₂₀)₂, —NO₂, —SR₂₀, —(C₁-C₈)alkyl, —O-(C₁-C₈)alkyl, —CON(R₂₀)₂, or —SO₃H;
A₁ to A₆ each independently is a group of Formula III, i.e., a pyridine or pyridine derivative moiety, or of Formula IV, i.e., pyrimidine or pyrimidine derivative moiety, linked to the ring structure of the complex of general Formula I via R₁₉ 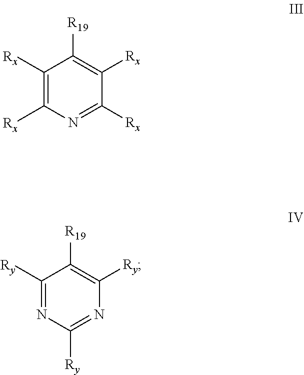
R₁₉ each independently is selected from a covalent bond, H₂C—CH₂, HC═CH, C≡C, N═N, HC═N, N═CH, H₂C—NH, HN—CH₂, —COO—, —CONH—, —CON(OH)—, —NR₂₀—, —Si(R₂₀)₂—, an alkylene optionally interrupted by one or more heteroatoms selected from O, S, or N, phenylene, biphenylene, a peptide moiety consisting of 3 to 5 amino acid residues, 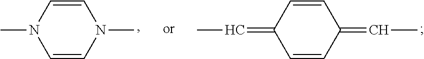
Rₓ and R_{y} each independently is selected from H, halogen, —OH, —N₃, —NO₂, —CN, —N(R₂₀)₂, —CON(R₂₀)₂, —COOR₂₀, —SR₂₀, —SO₃H, —CH═CH-pyridyl, —(C₁-C₁₀)alkyl, —(C₂-C₁₀)alkenyl, —(C₂-C₁₀)alkynyl, —(C₁-C₁₀)alkoxy, cycloalkyl, heterocycloalkyl, aryl, heteroaryl, protected carboxyl, or protected amino, wherein the (C₁-C₁₀)alkyl, (C₂-C₁₀)alkenyl, (C₂-C₁₀)alkynyl, cycloalkyl, heterocycloalkyl, aryl, or heteroaryl may optionally be substituted with halogen, —OR₂₀, —COR₂₀, —COOR₂₀, —OCOOR₂₀, —OCON(R₂₀)₂, —(C₁-C₈)alkylene-COOR₂₀, —CN, —N(R₂₀)₂, —NO₂, —SR₂₀, —(C₁-C₈)alkyl, —O-(C₁-C₈)alkyl, —CON(R₂₀)₂, or —SO₃H; and
R₂₀ each independently is H, (C₁-C6)alkyl, or aryl; or wherein
the polypyridyl complex is represented by Formula II: 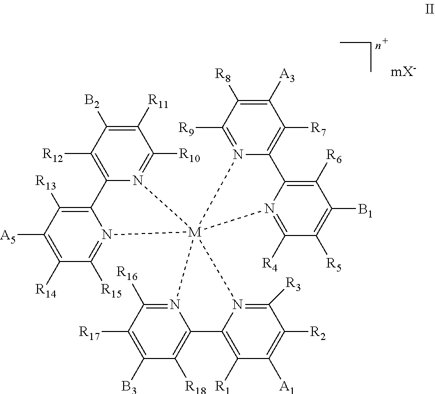
Wherein
M is a transition metal selected from Fe, Os, Ru, Co, Ni, Mn, Cu, Zn, Ti, Cr, Rh, or Ir;
n is the formal oxidation state of M, wherein n is 0-6;
X is a counter ion;
m is a number ranging from 0 to 6;
R₁ to R₁₈ each independently is selected from H, halogen, —OH, —N₃, —NO₂, —CN, —N(R₂₀)₂, —CON(R₂₀)₂, —COOR₂₀, —SR₂₀, —SO₃H, —CH═CH-pyridyl, —(C₁-C₁₀)alkyl, —(C₂-C₁₀)alkenyl, —(C₂-C₁₀)alkynyl, —(C₁-C₁₀)alkoxy, cycloalkyl, heterocycloalkyl, aryl, heteroaryl, wherein the (C₁-C₁₀)alkyl, (C₂-C₁₀)alkenyl, (C₂-C₁₀)alkynyl, cycloalkyl, heterocycloalkyl, aryl, or heteroaryl may optionally be substituted with halogen, —OR₂₀, —COR₂₀, —COOR₂₀, —OCOOR₂₀, —OCON(R₂₀)₂, —(C₁-C₈)alkylene-COOR₂₀, —CN, —N(R₂₀)₂, —NO₂, —SR₂₀, —(C₁-C₈)alkyl, —O-(C₁-C₈)alkyl, —CON(R₂₀)₂, or —SO₃H;
A₁, A₃, and A₅ each independently is a group of Formula III, i.e., a pyridine or pyridine derivative moiety, or of Formula IV, i.e., pyrimidine or pyrimidine derivative moiety, linked to the ring structure of the complex of general Formula II R₁₉ 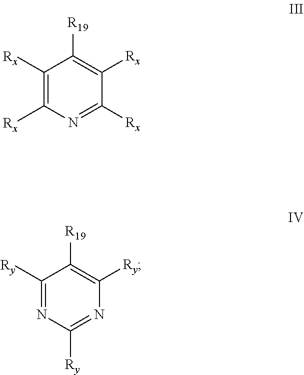
R₁₉ each independently is selected from a covalent bond, H₂C—CH₂, cis/trans HC═CH, C≡C, N═N, HC═N, N═CH, H₂C—NH, HN—CH₂, —COO—, —CONH—, —CON(OH)—, —NR₂₀—, —Si(R₂₀)₂—, an alkylene optionally interrupted by one or more heteroatoms selected from O, S, or N, phenylene, biphenylene, a peptide moiety consisting of 3 to 5 amino acid residues, 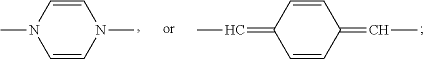
Rₓ and R_{y} each independently is selected from H, halogen, —OH, —N₃, —NO₂, —CN, —N(R₂₀)₂, —CON(R₂₀)₂, —COOR₂₀, —SR₂₀, —SO₃H, —CH═CH-pyridyl, —(C₁-C₁₀)alkyl, —(C₂-C₁₀)alkenyl, —(C₂-C₁₀)alkynyl, —(C₁-C₁₀)alkoxy, cycloalkyl, heterocycloalkyl, aryl, heteroaryl, protected carboxyl, or protected amino, wherein the (C₁-C₁₀)alkyl, (C₂-C₁₀)alkenyl, (C₂-C₁₀)alkynyl, cycloalkyl, heterocycloalkyl, aryl, or heteroaryl may optionally be substituted with halogen, —OR₂₀, —COR₂₀, —COOR₂₀, —OCOOR₂₀, —OCON(R₂₀)₂, —(C₁-C₈)alkylene-COOR₂₀, —CN, —N(R₂₀)₂, —NO₂, —SR₂₀, —(C₁-C₈)alkyl, —O-(C₁-C₈)alkyl, —CON(R₂₀)₂, or —SO₃H;
B₁ to B₃ each independently is selected from H, halogen, —OH, —N₃, —NO₂, —CN, —N(R₂₀)₂, —CON(R₂₀)₂, —COOR₂₀, —SR₂₀, —SO₃H, —CH═CH-pyridyl, —(C₁-C₁₀)alkyl, —(C₂-C₁₀)alkenyl, —(C₂-C₁₀)alkynyl, —(C₁-C₁₀)alkoxy, cycloalkyl, heterocycloalkyl, aryl, heteroaryl, protected carboxyl, or protected amino, wherein the (C₁-C₁₀)alkyl, (C₂-C₁₀)alkenyl, (C₂-C₁₀)alkynyl, cycloalkyl, heterocycloalkyl, aryl, or heteroaryl may optionally be substituted with halogen, —OR₂₀, —COR₂₀, —COOR₂₀, —OCOOR₂₀, —OCON(R₂₀)₂, —(C₁-C₈)alkylene-COOR₂₀, —CN, —N(R₂₀)₂, —NO₂, —SR₂₀, —(C₁-C₈)alkyl, —O-(C₁-C₈)alkyl, —CON(R₂₀)₂, or —SO₃H; and
R₂₀ each independently is H, (C₁-C₆)alkyl, or aryl.

13. (canceled)

14. The method of claim 12, wherein the pyridyl complex is represented by one of the following formulas, or by a mixture of the following formulas, or by a combination of the following formulas with other pyridyl complexes: 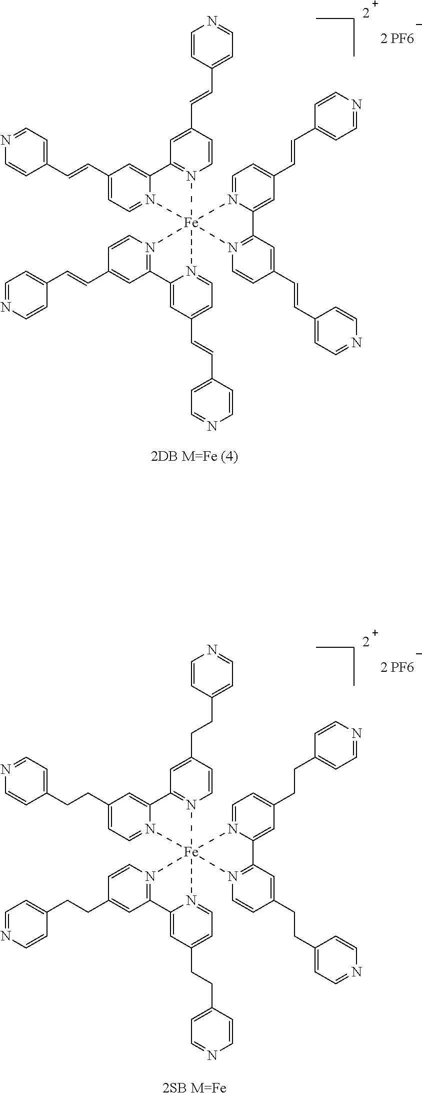 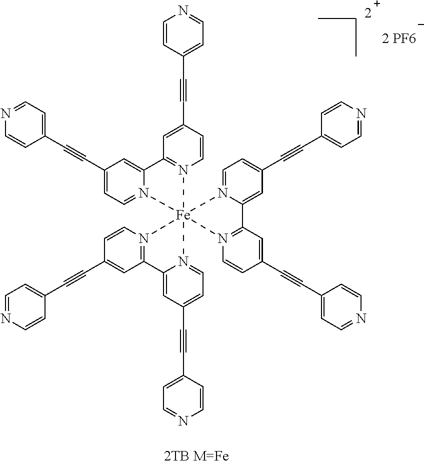
or wherein
the pyridyl complex is represented by one of the following formulas, or by a mixture of the following formulas, or by a combination of the following formulas with other pyridyl complexes:

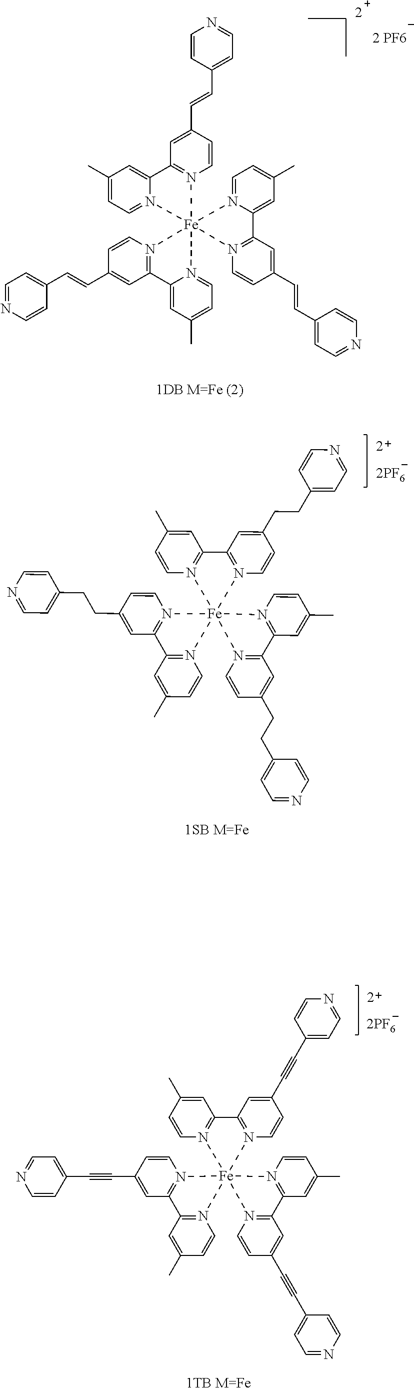

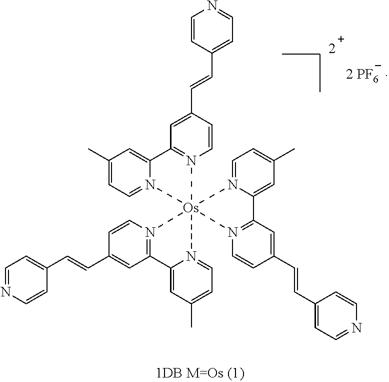

15. (canceled)

16. The method of claim 1, wherein said substrate or a portion thereof is conductive, and optionally wherein the substrate is selected from the group consisting of ITO, FTO, ITO or FTO-coated polyethylene terephthalate, ITO-coated glass or quartz, and FTO coated glass or quartz.

17. (Canceled)

18. The method of claim 1, wherein said substrate or portion thereof is transparent in at least a portion of the UV range, in at least a portion of the visible range or in a combination thereof, and optionally wherein said substrate or portion thereof is transparent throughout the visible range.

19. (canceled)

20. The method of claim 1, wherein:
the metal linker comprising a metal ion is a mixture of different linkers; or wherein the polypyridyl complex is a mixture of two or more polypyridyl complexes; or a combination thereof.

21. (canceled)

22. The method of claim 1, wherein the step of applying a linker comprises applying the linker by spraying a solution comprising said linker, and wherein the step of applying at least one metal-coordinated organic complex comprises applying the metal-coordinated organic complex by spraying a solution comprising said metal-coordinated organic complex, and wherein said solutions comprise a solvent.

23. The method of claim 22, wherein said solvent is selected from the group consisting of THE, alcohols, ethers, esters, halogenated solvents, hydrocarbons, ketones, or a mixture thereof.

24. (canceled)

25. The method of claim 22, wherein the concentration of said linker in said solution and the concentration of said metal-coordinated organic complex in said. solution ranges between 0.1 mM and 10 mM.

26. An electrochromic (EC) device comprising a substrate and comprising at least one layer of a linker and at least one layer of a metal-coordinated organic complex, said device is produced by a method comprising:
a. providing a substrate;
b. applying a linker comprising a metal ion to said substrate by spray-coating, thus forming a linker layer on said substrate;
c. applying a metal-coordinated organic complex to said linker layer by spray coating, thus forming a layer of metal-coordinated organic complex on said linker layer;
d. optionally repeating steps b and c;
wherein the device further comprising a power supply and electrical connections said electrical connections connecting said device to the power supply wherein:
a first connection connecting said substrate to a first pole of said power supply;
a second connection connecting said metal-coordinated organic complex layer directly or through intermediate layer(s) to a second pole of said power supply.

27. The EC device of claim 26, wherein the thickness of the linker/organic layers measured perpendicular to the substrate surface ranges between 10 nm and 1 mm, or between 10 nm and 1000 nm or between 10 nm and 250 nm or between 50 nm and 250 nm or between 100 nm and 300 nm, and wherein the dimensions of the device parallel to the substrate surface comprise length and width ranging between 1 mm and 10 m, and the thickness of the device including the substrate, measured perpendicular to the substrate surface is ranging between 1 μm and 1 cm.

28. (canceled)

29. The EC device of claim 26, wherein said metal-coordinated organic complex comprises one type of metal ion, or wherein said metal-coordinated organic complex comprises at least two types of metal ions, and optionally wherein said at least two types of metal ions comprise metal ions selected from Fe, Os, Ru, Co, Ni, Mn, Cu, Zn, Ti, Cr, Rh, or Ir.

30-31. (canceled)

32. The EC device of claim 29, wherein said metal-coordinated organic complex is a polypyridine complex comprising two types of metal ions, said two types are Fe and Os ions or Fe and Ru ions or Ru and Os ions. 33, (Original) The EC device of claim 26, having a contrast ratio between an oxidized and a reduced state of at least 10%, at least 20%, at least 30%, at least 40%, at least 50% or at least 60%, or a contrast ratio ranging between 10% and 20%, between 10% and 50%, between 25% and 50%, between 10% and 40% or between 10% and 70%.

34. The EC device of claim 26, able to retain at least 90% of its maximum contrast ratio after 1000 switching cycles between oxidized and reduced state(s).

35-36. (canceled)

37. The EC device of claim 26, wherein said intermediate layers comprise an electrolyte, a storage layer, a spacer or any combination thereof.

38. A smart window comprising the device of claim 26, wherein said substrate is transparent in the visible-light range and wherein the lateral length and width of said window measured parallel to the largest surface of said substrate is ranging between 1 cm to 10 m.

39. An optical switch, a memory device or an encoder comprising:
the device of claim 26;
an optical detector.

40. The optical switch, the memory device or the encoder of claim 39, wherein said substrate is transparent in at least a portion of the visible-light range.

41. The optical switch, the memory device or the encoder of claim 39, further comprising a light source.

42. A display comprising the device of claim 26.

43. The display of claim 42, wherein said intermediate layers comprise an electrolyte and wherein said electrolyte is a solid electrolyte.

44. The display of claim 42, wherein said display comprises multiple electrochromic devices such that each electrochromic device forms one or more pixel(s) in said display.

45-54. (canceled)
